SEQUENCE LISTING (21 sequences)

SEQ ID NO: 1 - DNA, 20 bp, artificial sequence
  forward primer for NAH subfamily
caaaarcacctgattyatgg

SEQ ID NO: 2 - DNA, 20 bp, artificial sequence
  reverse primer for NAH subfamily
ayrcgrgsgacttctttcaa

SEQ ID NO: 3 - DNA, 19 bp, artificial sequence
  forward primer for TOD subfamily
accgatgargayctgtacc

SEQ ID NO: 4 - DNA, 20 bp, artificial sequence
  reverse primer for TOD subfamily
cttcggtcmagtagctggtg

SEQ ID NO: 5 - DNA, 22 bp, artificial sequence
  forward primer for TOL subfamily
tgaggctgaaactttacgtaga

SEQ ID NO: 6 - DNA, 19 bp, artificial sequence
  reverse primer for TOL subfamily
ctcacctggagttgcgtac

SEQ ID NO: 7 - DNA, 19 bp, artificial sequence
  forward primer for BPH1 subfamily
ggacgtgatgctcgaycgc

SEQ ID NO: 8 - DNA, 22 bp, artificial sequence
  reverse primer for BPH1 subfamily
tgttsggyacgttmaggcccat

SEQ ID NO: 9 - DNA, 20 bp, artificial sequence
  forward primer for BPH2 subfamily
gacgcccgcccctatatgga

SEQ ID NO: 10 - DNA, 21 bp, artificial sequence
  reverse primer for BPH2 subfamily
agccgacgttgccaggaaaat

SEQ ID NO: 11 - DNA, 19 bp, artificial sequence
  forward primer for BPH3 subfamily
ccgggagaacggcaggatc

SEQ ID NO: 12 - DNA, 19 bp, artificial sequence
  reverse primer for BPH3 subfamily
tgctccgctgcgaacttcc

SEQ ID NO: 13 - DNA, 20 bp, artificial sequence
  forward primer for BPH4 subfamily
aaggccggcgacttcatgac

SEQ ID NO: 14 - DNA, 21 bp, artificial sequence
  forward primer for RMO subfamily
tctcvagcatycagacvgacg

SEQ ID NO: 15 - DNA, 20 bp, artificial sequence
  reverse primer for RMO subfamily
ttktcgatgatbacrtccca

SEQ ID NO: 16 - DNA, 22 bp, artificial sequence
  forward primer for RDEG subfamily
tytcvagcathcaracvgayga

SEQ ID NO: 17 - DNA, 20 bp, artificial sequence
  reverse primer for RDEG subfamily
ttdtcgrtratbacrtccca

SEQ ID NO: 18 - DNA, 21 bp, artificial sequence
  forward primer of PHE subfamily
gtgctgacsaayctgytgttc

SEQ ID NO: 19 - DNA, 17 bp, artificial sequence
  reverse primer for PHE subfamily
cgccagaaccayttrtc

SEQ ID NO: 20 - DNA, 20 bp, artificial sequence
  primer for amplifying 16S rRNA gene
actcctacgggaggcagcag

SEQ ID NO: 21 - DNA, 17 bp, artificial sequence
  primer for amplifying PRUN518R gene
attaccgcggctgctgg

(19) United States
(12) Patent Application Publication
(10) Pub. No.: US 2004/0161767 A1
(43) Pub. Date: Aug. 19, 2004

(54) Detection and quantification of aromatic oxygenase genes by real-time PCR

(76) Inventors: Brett R. Baldwin, Brownsburg, IN (US); Loring F. Nies, Attica, IN (US); Cindy H. Nakatsu, West Lafayette, IN (US)

Correspondence Address: Gregory B. Coy, Woodard, Emhardt, Moriarty, McNett & Henry LLP, Bank One Center/Tower, 111 Monument Circle, Suite 3700, Indianapolis, IN, 46204-5137, US

(21) Appl. No.: 10/611,089

(22) Filed: Jun. 30, 2003

Related U.S. Application Data
(60) Provisional application No. 60/392,360, filed on Jun. 28, 2002.

(51) Int. Cl. C12Q 1/68; C12P 19/34

(52) U.S. Cl. 435/6; 435/91.2

(57) ABSTRACT

The present invention provides a direct manner of assessing the bioremediation potential of microbes in a soil sample by detecting and enumerating the microbes that have the necessary functional genes to metabolize specific pollutants. In particular, the present invention provides novel compositions and methods for analyzing a sample containing a diverse population of microbes to detect and quantify the presence of specific functional aromatic pollutant oxygenase genotypes in the population. The detection and quantification of genotypes in accordance with the invention provides a manner in which the bioremediation potential of the sample can be assessed in a reliable manner. Quantification is achieved in accordance with the invention by quantitative PCR amplification using primers that are constructed or selected to amplify target regions identified to be included in conserved regions of genes from diverse microbial species that have aromatic pollutant metabolism functionality.

### REFERENCE TO RELATED APPLICATION

This application claims priority to prior U.S. Provisional Patent Application Serial No. 60/392,360, filed Jun. 28, 2002, which is incorporated herein by reference in its entirety.

### BACKGROUND OF THE INVENTION

The present invention relates to the field of research relating to microbial bioremediation of polluted soil or water. More particularly, the invention relates to the use of quantitative Polymerase Chain Reaction (PCR) to detect and quantify particular genotypes in a soil or water sample to assess the bioremediation potential of microbes in the sample.

As a background to the invention, a great deal of attention has been given in recent years to the bioremediation of polluted soil, lakes, streams, groundwater and the like. Environmental pollutants such as, for example, petroleum, that are leaked or otherwise released into the environment are gradually degraded by microbial activities in the environment and naturally cleansed over time. Bioremediation refers generally to the conversion of harmful pollutants to innocuous compounds by microbes, either microbes already present in the soil or water, or microbes that are introduced into the soil or water for the express purpose of promoting bioremediation.

On a global scale, estimates suggest that the annual input of petroleum to the environment from anthropogenic sources is between 1.7 and 8.8 million metric tons. It has been reported that, in the U.S. alone, approximately 295,000 sites are believed to be contaminated by petroleum products. Reports indicate that the majority of these sites (as many as 165,000) result from leaks in underground storage tanks (USTs) commonly used to store petroleum products like gasoline and diesel fuel.

Although petroleum products are complex mixtures comprised of hundreds of compounds, the aromatic hydrocarbons are generally the contaminants of principal concern, and may comprise as much as 20% of petroleum products like unleaded gasoline. Exposure to certain members of the aromatic family of compounds has been determined to have adverse health effects including carcinogenicity and depression of the central nervous system. Furthermore, the mono-aromatic compounds like benzene, toluene, and xylenes (BTX) are relatively soluble, fueling concerns regarding the migration of these pollutants into the groundwater, which poses a substantial threat to human health and environment. Due to their toxicity and mobility in the environment, BTX concentrations are often used to establish cleanup requirements at contaminated sites.

In recent years, bioremediation, i.e., biodegradation of pollutants by microbial metabolism, has been increasingly used as a treatment technology for petroleum contaminated sites. Bioremediation is particularly desirable as a treatment option compared to other traditional methods of treatment or clean-up due to its broad applicability, potentially low costs, and its ability to convert hazardous pollutants into innocuous compounds, unlike traditional methods of treatment and disposal which merely transfer the pollutant from one medium to another.

Concerns regarding aromatic pollutants have prompted intense research into the biodegradation of these pollutants by soil microorganisms. The aromatic ring is one of the most widely distributed chemical structures in nature. Consequently organisms capable of utilizing aromatic compounds as sole carbon and energy sources have been found to be nearly ubiquitous in soil ecosystems. Providing site conditions that allow stimulation of aromatic hydrocarbon-degrading bacteria from the indigenous population therefore should permit successful bioremediation of these sites. However, there is no accurate method for analyzing and evaluating the constitution and fluctuation of the microbial population that bears the burden of natural cleanup at a contaminated site. Thus, it is currently difficult to evaluate the efficacy of bioremediation as a treatment.

Metabolic pathways have been discovered which lead to the biodegradation of many environmentally important aromatic compounds including mono-aromatics, biphenyl, and polycyclic aromatic hydrocarbons (PAHs). Further research has examined effector compounds and the details of regulation. DNA sequences have been determined for some of the aromatic catabolic genes and are available on databases such as GenBank. Comparison of the gene and amino acid sequences have provided insight into enzyme specificity and pathway synthesis.

Overall, biodegradation of aromatic hydrocarbons is often viewed as modular in nature with a variety of upper pathways converging on a limited number of common intermediates which are further metabolized by a few well-conserved pathways. Although distinct pathways have been discovered for catabolism of different aromatic hydrocarbons, these pathways often proceed by a common method composed of four reactions: (1) Biodegradation is initiated by an oxygenase enzyme which incorporates molecular oxygen into the aromatic ring forming a cis-dihydrodiol intermediate. (2) A dehydrogenase enzyme catalyzes the production of the corresponding diol. (3) Cleavage is mediated by a meta-cleavage dioxygenase. (4) The final step is hydrolysis forming an unsaturated aliphatic acid from one of the aromatic rings.

With respect to biodegradation of toluene (and other alkyl-substituted benzenes), this process is initiated in two ways: oxidation of the methyl (alkyl-) group or direct oxygenase attack on the aromatic ring at a variety of positions. Bacterial strains have been described which produce ring hydroxylating-monooxygenase enzymes capable of introducing oxygen at the ortho, meta, or para positions. For example, toluene metabolism can be initiated by monooxygenase attack at the ortho position as demonstrated by Burkholderia cepacia G4. Toluene biodegradation by P. pickettii PK01 is initiated by a toluene monooxygenase with relatively broad specificity targeting the meta ring position. P. mendocina KR1 has been reported to initiate metabolism of toluene by monooxygenation of the ring at the para position. In addition to toluene monooxygenases which hydroxylate the aromatic ring at discrete positions, other toluene monooxygenases have been characterized which hydroxylate at multiple positions yielding a mixture of products. The toluene/o-xylene monooxygenase from Pseudomonas stutzeri OX1 has been shown to exhibit low regiospecificity uncommon among oxygenases, which often display broad substrate specificity, but usually exhibit narrow regiospecificity. Toluene monooxygenase from P. stutzeri OX1 have been reported to hydroxylate toluene at each position producing o-, m-, and p-cresol. Furthermore, P. stutzeri OX1 oxidizes o-xylene to both 2,3- and 3,4-dimethylphenol. Toluene dioxygenase catalyzes the incorporation of both atoms of molecular oxygen into the aromatic ring forming a cis-dihydrodiol that is subsequently dehydrogenated to 3-methylcatechol. This intermediate is subject to cleavage mediated by a second dioxygenase. Numerous organisms have also been described which catabolize toluene by methyl group oxidation encoded on TOL plasmids similar to the archetype TOL plasmid of Pseudomonas putida mt-2.

Among the toluene-utilizing strains characterized, Burkholderia sp. strain JS150 has been given special attention. The majority of the early work with this strain focused on toluene dioxygenase; however, strain JS150 has been reported to synthesize multiple upper and lower pathways for the oxidation of substituted benzenes. In all, Burkholderia sp. JS150 has been found to express three distinct monooxygenases for the initial oxidation of the nucleus of aromatic compounds. Although not as well characterized, the toluene-4-monooxygenase is believed to catalyze oxidation of toluene and 4-methylphenol.

Benzene biodegradation can be initiated by dioxygenase attack or monooxygenation of the ring to produce catechol. Further biodegradation of the catechol is mediated by the ortho- or meta-cleavage routes depending on the organism. The m- and p-xylene isomers are degraded by TOL plasmid containing organisms to produce methyl catechol which are further metabolized by meta cleavage. It has been reported that direct dioxygenase attack at the aromatic moiety of m- and p- xylene will yield cis-dihydrodiols and corresponding substituted catechols which are not usually degraded further. The xylene isomers also serve as substrates for the ring-hydroxylating toluene monooxygenase mediated pathways described earlier.

Aerobic metabolism of naphthalene is encoded on two operons. It has been reported that the upper pathway is needed to convert naphthalene to salicylate while the lower pathway is responsible for the conversion of salicylate to central metabolites. Regulation of the two operons is coordinated by a single regulator protein. Salicylate produced by low level constitutive expression of the upper pathway has been reported to induce expression of both operons in conjunction with the regulator protein NahR. Conversion of naphthalene to gentisate has also been reported. The naphthalene catabolic pathway is also responsible for the biodegradation of aromatic compounds in addition to naphthalene.

Like naphthalene, aerobic metabolism of biphenyl is divided into upper and lower pathways. The upper pathway will convert biphenyl to benzoate, which is converted via meta-cleavage to central metabolites in the lower pathway. Regulation of biphenyl catabolic pathways is still poorly understood even considering the attention garnered by polychlorinated biphenyl (PCBs). Unsubstituted biphenyl and for some strains mono-aromatic compounds like ethylbenzene are known to induce expression of the pathway.

While aromatic hydrocarbon metabolism is becoming better understood, presently little is known about the ecology of biodegradation of aromatic compounds at the field scale, mainly because little effort has been made to document biodegradation even at sites undergoing bioremediation as the treatment technology. Research conducted to date has provided much information regarding the metabolic pathways of many aromatic pollutants, but these experiments, by necessity, have focused on individual compounds and pure bacterial cultures to examine the fundamentals of aromatic hydrocarbon catabolism, and have not significantly advanced understanding of bioremediation in the field. Often groundwater BTEX concentrations are the only measurements used to evaluate the efficacy of bioremediation in the field. While such data is critical for documenting remediation, it does not determine the actual treatment mechanism. Once released into the environment, aromatic hydrocarbons are subject to physical as well as biological processes. Adsorption to soil organic matter, volatilization, and dilution will reduce groundwater BTEX concentrations, but do not remove the pollutants from the environment. Converging lines of evidence must be used to document biodegradation in the field. As is usually done, concentrations of target pollutants must be periodically monitored to determine contaminant removal and assess risk of migration to sensitive receptors. Samples can also be taken at monitoring events to measure geochemical parameters indicative of biological activity. For petroleum hydrocarbons, measuring electron acceptor concentrations such as dissolved oxygen, NO₃⁻, Fe²⁺, and SO₄²⁻ as well as their reduced products inside and outside of the contaminate plume provide indirect evidence of biodegradation. Reduction of NO₃⁻, Fe²⁺, and SO₄²⁻ also increase alkalinity within the plume indicating bioremediation is occurring. While such measurements provide strong indirect evidence, the most direct approach would be to quantify the organisms responsible for the biodegradation of the target pollutants. For example, elevated populations of BTEX-degraders, relative to uncontaminated areas, would be strong evidence of biodegradation.

One approach for identifying and quantifying microbes in a contaminated sample is by isolating and culturing microbes from a sample. Traditional cultivation-based methods however, have not proven to be a suitable approach for measuring bioremediation potential. Conventional culture and counting techniques have the drawback of requiring significant labor time and effort, and a long culture time for detection of specific microorganisms of interest. In addition, very few of the microorganisms that live in the natural environment can be detected by conventional isolation and cultivation techniques, and such techniques are therefore believed to drastically underestimate the number of aromatic hydrocarbon-degrading bacteria in a sample. Specifically, the percentage of microorganisms that can be isolated and cultured with such techniques has been reported to be no more 1% in comparison to the total number of microorganisms that can be quantified through a direct microscopic counting. Therefore, difficulty in analysis of the population structure and fluctuations of the microbial community that live in an environment, and of the behavior of specific microorganisms is a major obstacle.

Our knowledge of aromatic catabolic pathways is partly bounded by the organisms used for study. Traditionally, culturing on selective media (spread plates or most probable number) with the contaminant of interest has been used to detect and enumerate bacteria with a particular catabolic phenotype. While some strains have been isolated directly, enrichment in batch culture is the most widely used technique for isolation of organisms with a particular phenotype. Not only will enrichment with a particular compound as a carbon source select for bacteria capable of utilizing it for growth, but enrichment will also select for the fastest-growing organisms under the enrichment conditions. Care must therefore be exercised during isolation to ensure that strains isolated are truly indicative of various populations with a given phenotype. Furthermore, as stated above, an overwhelming majority of these organisms are not readily cultivated. Therefore, enumeration based on culturing techniques will vastly underestimate the total population with the desired phenotype. In addition, the culturable fraction may not be a representative sample of the bacterial community members capable of growth on that particular substrate.

To avoid biases associated with culturing, molecular techniques based on detection of specific catabolic genes can be used to assess biodegradation potential more directly. In this regard, a variety of molecular methods including DNA hybridization, polymerase chain reaction (PCR), cloning and nucleotide sequencing analysis can be used to detect targeted genotypes from a pool of unknown DNA extracted directly from environmental microorganism samples. Such methods are allowing the gradual elucidation of information regarding the diversity of microorganisms and the microbial population structure in a natural environment. While probing has been successful for detecting naphthalene, toluene, biphenyl, and chlorocatechol metabolic genes, potential problems have arisen. At low stringency probes may cross-hybridize producing false positive results. Conversely at high stringency, related but not identical genes may be excluded from detection. PCR amplification of aromatic catabolic genes has also been used to detect specific genes in environmental samples and may avoid false positive and false negative results given by direct hybridization. However, the usefulness of information obtained using such methods is limited because molecular techniques have heretofore been necessarily limited to the detection of microbial species that have already been identified, isolated and characterized. Thus, only specific, known genes are analyzed. Because little is known of most microbial species expected to be present in the field, such information is not considered to be particularly reliable as a bioremediation assessment tool.

Despite the fact that aromatic compounds are biodegradable, and despite the advantages of bioremediation over traditional technologies, bioremediation, and in particular monitored natural attenuation (MNA), often meet with skepticism from regulatory agencies and the public for two main reasons. First, no effort is usually made to ensure that contaminant loss at the site is due to biodegradation. At most sites quarterly monitoring of groundwater BTEX concentrations and groundwater elevation are the only data that are required for model inputs to delineate the plume and assess risk. No direct measurements are usually made to determine whether decreases in concentrations are actually due to biodegradation. Increasing acceptance of bioremediation as a treatment option relies on demonstrating that contaminant loss is due to biodegradation. Second, little is currently known regarding biodegradation of aromatic hydrocarbons in complex petroleum mixtures observed in the field. Substrate interactions and microbial population dynamics can have dramatic effects on biodegradation in the field. Increasingly researchers are using molecular methods to investigate these issues and document the presence of catabolic genotypes in the environment. DNA hybridization has been reported to be successful for detecting naphthalene, toluene, biphenyl, and chlorocatechol metabolic genes. In addition, PCR amplification of a fragment of catechol 2,3-dioxygenase has been studied for use as a measure of biodegradation potential at contaminated sites. These efforts, however, have not yielded a suitable manner of assessing bioremediation potential in the field.

In view of the above background, and other considerations, it is apparent that there is a continuing need for further developments in the field of assessing bioremediation potential in a polluted soil or water sample. In particular, there is a need for further advancement in the development of methods for enumerating genotypes in a contaminated soil or water sample that are responsible for aromatic hydrocarbon metabolism. Such methods would provide evidence that degradation, as opposed to a physical process like dilution, is responsible for contaminant loss. The present invention addresses these needs, and further provides related advantages.

### SUMMARY OF THE INVENTION

Although efforts have been made to gauge the bioremediation activity of microbes in soil using indirect methods, to date, there have been developed no suitable protocols for detecting and enumerating microbes in a sample that correlates to the overall ability of the microbes present to metabolize the pollutants. The present invention provides a more direct manner of assessing the bioremediation potential of microbes in a soil sample by detecting and enumerating the microbes that have the necessary genes to metabolize specific pollutants. The invention provides methods and compositions for detecting and quantifying genotypes responsible for the biodegradation of target aromatic compounds in site samples. Thus, in contrast to protocols that quantify a preselected microbe species in a sample, the present invention allows quantification of functional genes that are known to be responsible for biodegradation of pollutants of interest in a sample, such as, for example, a sample from a petroleum-contamination site. The invention provides a novel manner of directly and accurately assessing the presence of genes that enable the bacterial community to biodegrade aromatic hydrocarbons.

Thus, the invention provides diagnostic methods and compositions for molecular-genetic analysis and evaluation of environments polluted or contaminated by noxious chemicals, particularly petroleum and/or petroleum components, and to bioremediation processes of the polluted or contaminated environments by microorganisms. The invention relates to methods for molecular genetic assessment of bioremediation potential provided by microorganisms with specific functions by detecting and quantifying the functional genes themselves in the sample. The present invention provides PCR protocols for the quantification of oxygenase genes responsible for the biodegradation of multiple priority pollutants at petroleum-contamination sites, including benzene, toluene, xylenes, biphenyl and naphthalene.

Tracking aromatic catabolic genes at contaminated sites aids bioremediation on two fronts: (1) periodic detection and quantification of aromatic catabolic genotypes, such as, for example, families or subfamilies of aromatic oxygenase genes, at contaminated sites provides direct evidence supporting biodegradation as the active mechanism for pollutant removal and (2) detection of aromatic catabolic genes provides insight into the selection of metabolic pathways in a real-world setting. In-situ microbial characterization protocols provided by the invention facilitate assessment of the impact of remediation technologies on indigenous microbial populations, provide more accurate assessment of intrinsic pollution degradation, and enhance studies of contaminated site ecology. Using the present invention, engineers can accurately assess the feasibility of bioremediation at sites undergoing monitored natural attenuation and optimize engineered systems to improve bioremediation performance.

It is an object of this invention to provide novel PCR primers and quantitative PCR protocols to detect and quantify aromatic oxygenase genes responsible for the biodegradation of several priority pollutants in environmental samples. It is another object of this invention to provide novel methods for making PCR primers useful for detecting and quantifying genes responsible for the biodegradation of aromatic pollutants in environmental samples. While the actual nature of the invention covered herein can only be determined with reference to the claims appended hereto, certain forms of the invention that are characteristic of the embodiments disclosed herein are described briefly as follows.

In one form of the invention, there is provided a method for assessing the bioremediation potential of a microbial community in a soil or water sample that includes: (1) providing a plurality of PCR primer sets, wherein each set corresponds to a distinct family or subfamily of functional aromatic oxygenase genes and is effective to selectively amplify target regions from diverse aromatic oxygenase genes in the corresponding family or subfamily; (2) providing a mixture of polynucleotides isolated from microbes present in a soil or water sample; (3) performing one or more quantitative PCR amplification reactions using the primer sets to quantify diverse aromatic oxygenase genes of each corresponding family or subfamily in the mixture; and (4) determining the bioremediation potential of microbes in the sample based upon results of the one or more quantitative PCR reactions. The sample can be, for example, a sample from a petroleum contaminated site.

The PCR analysis can be real-time quantitative PCR analysis. The real-time quantitative PCR analysis is preferably of the type that is performed using a double stranded DNA-binding dye, such as, for example, a SYBR Green dye. Alternatively, the real-time quantitative PCR analysis can be of the type that is performed using probes, such as, for example, molecular beacons, hybridization probes and hydrolysis probes, which probes are effective to hybridize to a polynucleotide segment of from about 10 to about 40 bases that is conserved in the members of each family or subfamily. In one preferred embodiment, the plurality of primer sets includes at least two primer sets, each of which is effective to selectively amplify a family or subfamily of functional aromatic oxygenase genes selected from the group consisting of naphthalene dioxygenase genes, toluene dioxygenase genes, xylene monooxygenase genes, biphenyl dioxygenase genes, toluene monooxygenase genes and phenol monooxygenase genes. For example, one or more of the following primer sets can be used to practice the invention: a set comprising a forward primer having the nucleotide sequence of SEQ ID NO: 1 and a reverse primer having the nucleotide sequence of SEQ ID NO: 2; a set comprising a forward primer having the nucleotide sequence of SEQ ID NO: 3 and a reverse primer having the nucleotide sequence of SEQ ID NO: 4; a set comprising a forward primer having the nucleotide sequence of SEQ ID NO: 5 and a reverse primer having the nucleotide sequence of SEQ ID NO: 6; a set comprising a forward primer having the nucleotide sequence of SEQ ID NO: 7 and a reverse primer having the nucleotide sequence of SEQ ID NO: 8; a set comprising a forward primer having the nucleotide sequence of SEQ ID NO: 9 and a reverse primer having the nucleotide sequence of SEQ ID NO: 10; a set comprising a forward primer having the nucleotide sequence of SEQ ID NO: 11 and a reverse primer having the nucleotide sequence of SEQ ID NO: 13; a set comprising a forward primer having the nucleotide sequence of SEQ ID NO: 12 and a reverse primer having the nucleotide sequence of SEQ ID NO: 13; a set comprising a forward primer having the nucleotide sequence of SEQ ID NO: 14 and a reverse primer having the nucleotide sequence of SEQ ID NO: 15; a set comprising a forward primer having the nucleotide sequence of SEQ ID NO: 16 and a reverse primer having the nucleotide sequence of SEQ ID NO: 17; and a set comprising a forward primer having the nucleotide sequence of SEQ ID NO: 18 and a reverse primer having the nucleotide sequence of SEQ ID NO: 19. In another embodiment, the method includes performing real-time quantitative PCR analysis of the mixture using each of the above-identified primer sets.

In certain preferred embodiments, at least one of the one or more quantitative PCR amplification reactions comprises a multiplex real-time quantitative PCR reaction. In one embodiment, a first primer set that is effective to selectively amplify a family or subfamily of phenol monooxygenase genes and a second primer set that is effective to selectively amplify a family or subfamily of naphthalene dioxygenase genes are used together to amplify diverse target regions in a multiplex real-time quantitative PCR reaction. The multiplex reaction can be performed, for example, using a primer set pair including a forward primer having the nucleotide sequence of SEQ ID NO: 18, a reverse primer having the nucleotide sequence of SEQ ID NO: 19, a forward primer having the nucleotide sequence of SEQ ID NO: 1 and a reverse primer having the nucleotide sequence of SEQ ID NO: 2. In another embodiment, a first primer set that is effective to selectively amplify a family or subfamily of xylene monooxygenase genes and a second primer set that is effective to selectively amplify a family or subfamily of toluene dioxygenase genes are used together to amplify diverse target regions in a multiplex real-time quantitative PCR reaction. The multiplex reaction can be performed, for example, using a primer set pair including a forward primer having the nucleotide sequence of SEQ ID NO: 5, a reverse primer having the nucleotide sequence of SEQ ID NO: 6, a forward primer having the nucleotide sequence of SEQ ID NO: 3 and a reverse primer having the nucleotide sequence of SEQ ID NO: 4. In yet another embodiment, a first primer set that is effective to selectively amplify a first subfamily of biphenyl dioxygenase genes and a second primer set that is effective to selectively amplify a second subfamily of biphenyl dioxygenase genes are used together to amplify diverse target regions in a multiplex real-time quantitative PCR reaction. The multiplex reaction can be performed, for example, using a primer set pair including a forward primer having the nucleotide sequence of SEQ ID NO: 9, a reverse primer having the nucleotide sequence of SEQ ID NO: 10, a forward primer having the nucleotide sequence of SEQ ID NO: 12 and a reverse primer having the nucleotide sequence of SEQ ID NO: 13.

In another form of the invention, there is provided a screening protocol for detecting and quantifying multiple families or subfamilies of functional aromatic oxygenase genes of diverse aromatic pollutant-degrading microbial species in a soil or water sample that includes: (1) providing a mixture of polynucleotides isolated from microbes present in a soil or water sample; and (2) performing quantitative PCR analysis of the mixture using a plurality of primer sets configured to selectively amplify different families or subfamilies of functional aromatic oxygenase genes. In one embodiment, a plurality of the primer sets are suitable for use together in a multiplex real-time PCR reaction. In another embodiment, each of the primer sets is used in separate real-time quantitative PCR reactions to separately quantify each corresponding family or subfamily of functional aromatic oxygenase genes.

The invention also provides a method of monitoring the bioremediation potential of a microbial community in a soil or water system contaminated with aromatic pollutants that includes: (1) providing a mixture of polynucleotides isolated from a soil or water sample corresponding to the system; and (2) performing quantitative PCR analysis of said mixture using a plurality of primer sets configured to selectively amplify target segments from corresponding families or subfamilies of aromatic oxygenase genes to provide a quantity value corresponding to aromatic oxygenase gene abundance in the sample. The aromatic oxygenase gene abundance correlates with the bioremediation potential of the sample. In certain preferred embodiments, the method further includes perturbing the system, waiting a period of time sufficient to allow the microbial community in the system to respond to said perturbing, and repeating the providing and performing to determine whether the bioremediation potential of the sample has changed. The quantitative PCR can be competitive, noncompetitive, kinetic, or combinations thereof. In one embodiment, the mixture of polynucleotides includes a mixture of RNA polynucleotides and the method includes performing quantitative RT-PCR on the RNA mixture using a plurality of primer sets made or selected in accordance with the invention. Amplification by RT-PCR provides a quantity value corresponding to aromatic oxygenase gene expression in the sample. The quantitative RT-PCR can be competitive, noncompetitive, kinetic, or combinations thereof.

In yet another form of the invention, there is provided a real-time Polymerase Chain Reaction (PCR) method for the selective detection and quantification of diverse families or subfamilies of aromatic oxygenase genes, each family or subfamily including a unique conserved region or a plurality of unique conserved sub-regions, the method including: (1) providing a mixture of polynucleotides isolated from a soil or water sample; (2) providing a plurality of primer sets configured to selectively amplify target segments from corresponding families or subfamilies of aromatic oxygenase genes and (3) performing quantitative PCR analysis of the mixture using the plurality of primer sets to provide a quantity value corresponding to aromatic oxygenase gene abundance in the sample. The real-time PCR method can optionally include one or more multiplex PCR reactions. The real-time PCR method can be an intercalator-based method or a probe-based method. In a preferred embodiment, the method is an intercalator-based method utilizing a double stranded DNA-binding dye. In addition, the PCR method can be a reverse transcriptase quantitative PCR method. In one embodiment, each of the primer sets selected for use in the PCR method is effective for amplifying a target segment from a different family or subfamily including aromatic oxygenase genes selected from the group consisting of a naphthalene dioxygenase genes, toluene dioxygenase genes, xylene monooxygenase genes, biphenyl dioxygenase genes, toluene monooxygenase genes and phenol monooxygenase genes. Examples of primer sets suitable for such use include the primers set forth in SEQ ID NOs: 1-19.

In yet another form of the invention, there are provided a plurality of exemplary primer sets that have been made in accordance with the invention for selective amplification of various genotypes. Excellent primer sets for use in accordance with the invention include: a set comprising a forward primer having the nucleotide sequence of SEQ ID NO: 1 and a reverse primer having the nucleotide sequence of SEQ ID NO: 2; a set comprising a forward primer having the nucleotide sequence of SEQ ID NO: 3 and a reverse primer having the nucleotide sequence of SEQ ID NO: 4; a set comprising a forward primer having the nucleotide sequence of SEQ ID NO: 5 and a reverse primer having the nucleotide sequence of SEQ ID NO: 6; a set comprising a forward primer having the nucleotide sequence of SEQ ID NO: 7 and a reverse primer having the nucleotide sequence of SEQ ID NO: 8; a set comprising a forward primer having the nucleotide sequence of SEQ ID NO: 9 and a reverse primer having the nucleotide sequence of SEQ ID NO: 10; a set comprising a forward primer having the nucleotide sequence of SEQ ID NO: 11 and a reverse primer having the nucleotide sequence of SEQ ID NO: 13; a set comprising a forward primer having the nucleotide sequence of SEQ ID NO: 12 and a reverse primer having the nucleotide sequence of SEQ ID NO: 13; a set comprising a forward primer having the nucleotide sequence of SEQ ID NO: 14 and a reverse primer having the nucleotide sequence of SEQ ID NO: 15; a set comprising a forward primer having the nucleotide sequence of SEQ ID NO: 16 and a reverse primer having the nucleotide sequence of SEQ ID NO: 17; and a set comprising a forward primer having the nucleotide sequence of SEQ ID NO: 18 and a reverse primer having the nucleotide sequence of SEQ ID NO: 19.

In another aspect of the invention, primer set pairs are provided for performing multiplex real-time quantitative PCR. In one embodiment a primer set pair includes a forward primer having the nucleotide sequence of SEQ ID NO: 18, a reverse primer having the nucleotide sequence of SEQ ID NO: 19, a forward primer having the nucleotide sequence of SEQ ID NO: 1, and a reverse primer having the nucleotide sequence of SEQ ID NO: 2. In another embodiment a primer set pair includes a forward primer having the nucleotide sequence of SEQ ID NO: 5, a reverse primer having the nucleotide sequence of SEQ ID NO: 6, a forward primer having the nucleotide sequence of SEQ ID NO: 3, and a reverse primer having the nucleotide sequence of SEQ ID NO: 4. In yet another embodiment, a primer set pair includes a forward primer having the nucleotide sequence of SEQ ID NO: 9, a reverse primer having the nucleotide sequence of SEQ ID NO: 10, a forward primer having the nucleotide sequence of SEQ ID NO: 12, a reverse primer having the nucleotide sequence of SEQ ID NO: 13.

In another aspect of the invention, there is provided a method for making a series of PCR primer sets for use in determining bioremediation potential of microbes in a sample to be analyzed, including: (1) identifying a plurality of aromatic pollutants for which bioremediation potential is to be determined; (2) preparing an alignment of functional aromatic oxygenase genes for each group of oxygenase genes having specificity for one of the pollutants; wherein each of the alignments includes genes from diverse species that encode oxygenase enzymes effective to oxygenate the corresponding aromatic pollutant; (3) identifying a region of each alignment comprising from about 50 to about 1000 bases that is substantially conserved or that includes two or more sub-regions that are substantially conserved in a plurality of the genes in the alignment; and (4) preparing a series of primer sets, each primer set corresponding to one alignment and comprising a forward primer of from about 10 to about 40 bases complementary to a nucleotide segment of a first strand of the region and a reverse primer of from about 10 to about 40 bases complementary to a nucleotide segment of a second strand of the region. The forward and reverse primers corresponding to each alignment span a target region in each of the plurality of genes, and each primer set is effective to amplify the target regions from the plurality of genes when present in the sample by quantitative PCR.

Further forms, embodiments, objects, features, and aspects of the present invention shall become apparent from the description contained herein.

### BRIEF DESCRIPTION OF THE FIGURES

FIG. 1 depicts a phylogeny of the alpha subunits of aromatic dioxygenases as discussed in the Examples. The tree was constructed using the Neighbor-Joining method and bootstrapping analysis. Symbols at branch points, e.g. D.1.A, designate type (D), family (2), and subfamily (A). Subfamilies of genes were used to perform alignments leading to the identification of PCR primer sets.

FIG. 2 depicts a phylogeny of alpha subunits of aromatic monooxygenases as discussed in the Examples.

FIG. 3 depicts a BPH4 amplification plot. (▪) 10⁷ copies rxn⁻¹, (□) 10⁶ copies rxn⁻¹, (♦) 10⁵ copies rxn⁻¹, (⋄) 10⁴ copies rxn⁻¹, (●) 10³ copies rxn⁻¹, and no template control ( [symbol] ).

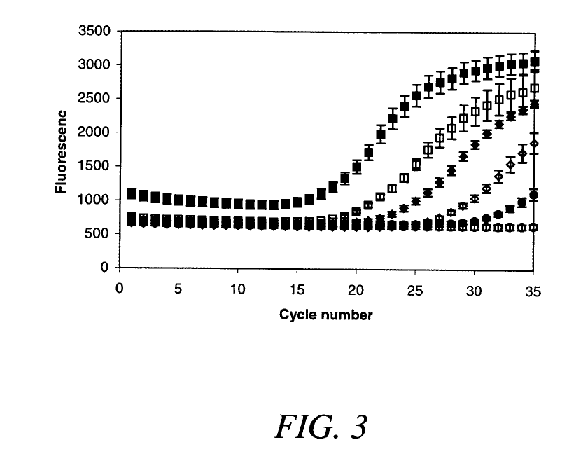

FIG. 4 depicts a NAH Calibration Curve as discussed in the Examples.

FIG. 5 depicts a layout of the Winamac site discussed in the Examples. (●) denotes monitoring well locations. Aromatic Hydrocarbons detected in samples from each well are noted in brackets. [B] Benzene, [T] Toluene, [E] Ethylbenzene, [X] xylenes, and [N] Naphthalene.

FIG. 6 depicts a layout of the Frankfort site discussed in the Examples. (●) denotes monitoring well locations. Aromatic Hydrocarbons detected in samples from each well are noted in brackets. [B] Benzene, [T] Toluene, [E] Ethylbenzene, [X] xylenes, and [N] Naphthalene.

FIG. 7 is a layout depicting detection of aromatic oxygenase genes at the Winamac Site. (●) Denotes monitoring well locations. The circled area estimates the contaminated plume based on previous BTEX concentrations. Detection of aromatic oxygenase genes are denoted by letters as follows: (P) PHE, (R) RMO, (T) TOL, and (N) NAH.

FIG. 8 is a layout depicting detection of aromatic oxygenase genes at the Frankfort Site. (●) Denotes monitoring well locations. The circle estimates the contaminated plume based on previous BTEX concentrations. Detection of aromatic oxygenase genes are denoted by letters as follows: (P) PHE, (R) RMO, (T) TOL, (TD) TOD, and (N) NAH, and (B) BPH4.

FIG. 9 depicts a plot of Log(Aromatic Oxygenase Copy Number g soil⁻¹) vs. Log(BTX). (♦) PHE and (□)RMO. Pearson product moment correlation coefficients (RSQ) were 0.14 and 0.22, respectively.

FIG. 10 is a graph representing average aromatic oxygenase gene copy numbers in contaminated and downgradient wells at the Frankfort Site. Vertically hashed bars are for wells with detectable BTX concentrations. Diagonally hashed bars are for the downgradient wells at the Frankfort site with non-detectable BTX concentrations. Error bars indicate one standard deviation.

### DESCRIPTION OF THE PREFERRED EMBODIMENTS

For the purpose of promoting an understanding of the principles of the invention, reference will now be made to preferred embodiments and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The present invention provides novel compositions and methods for analyzing a sample containing a diverse population of microbes to detect and quantify specific genotypes in the population. The detection and quantification of genotypes in accordance with the invention provides a manner in which the bioremediation potential of the sample can be assessed in a reliable manner. As used herein, the term “genotype” refers to a group of genes in a sample that share a specific function and a specific genetic constitution, such as, for example, genes having one or more conserved regions that encode oxygenase enzymes effective to oxygenate a specific aromatic pollutant. For purposes of describing the invention, genotypes are also referred to as gene “families” or “subfamilies.” The present invention provides a novel approach for the quick and accurate quantification of a genotype in a sample without the need for identifying the various species present in the sample that include the genotype. Quantification is achieved in accordance with the invention by quantitative PCR amplification using primers that are constructed or selected to amplify target regions that are included in regions of genes, such as, for example, regions of from about 50 to about 1000 bases, that are substantially conserved or that include two or more sub-regions that are substantially conserved, in a plurality of genes from diverse microbial species, i.e., species that have similar aromatic pollutant metabolism functionality. Indeed, compositions and methods provided by the present invention are believed to be effective to provide useful protocols for quantification of genotypes in a sample even where one or more of the microbes including the genotype in a given sample may have not been previously characterized and/or are not known.

The invention finds particularly advantageous use in the field of bioremediation of soil that has been contaminated with aromatic pollutants. As discussed above, a variety of microbial species have been identified that effectively metabolize aromatic pollutants such as, for example, naphthalene, toluene, xylene, biphenyl and phenol, and progress has been made in many cases to elucidate metabolic pathways used by such organisms, and to identify aromatic catabolic genes that participate in the metabolic pathways. In this regard, it is generally accepted that oxygenases play a key role in the aerobic metabolism of aromatic hydrocarbons. Indeed, the function of aromatic oxygenases is believed to be the rate-limiting step in aromatic pollutant biodegradation. In addition to initiating biodegradation of compounds ranging from benzene to phenanthrene, aromatic oxygenases, and the ∝ subunit of the oxygenases in particular, are believed to be responsible for the overall specificity of the pathways.

To optimize a bioremediation system, operating variables can be effectively evaluated by quantifying the genotypes in the sample that function to metabolize a given pollutant. In accordance with the present invention, genotypes are quantified by quantitative PCR protocols using primers constructed to selectively amplify specific genotypes, irrespective of the identity of the microbial species present. More particularly, PCR primers are constructed in accordance with the invention to hybridize, under PCR hybridization conditions, to a polynucleotide region that is conserved among substrate-specific oxygenase genes of diverse microbial species.

As such, the present invention provides PCR primers, methods for making PCR primers, and quantitative PCR (Q-PCR) protocols that are useful for detecting specific aromatic catabolic genotypes without excluding related but uncharacterized genes. Using primers designed to amplify target regions in such conserved regions, uncharacterized members of a family or subfamily are less likely to be excluded from detection. The quantification of genotypes in accordance with the invention provides useful information regarding the bioremediation potential of microbes in a sample, and can be used as an important factor in gauging the effect of bioremediation in the field. Quantification in accordance with the invention can also be used as a quick test to assess the effect on bioremediation of various soil or water amendments or other conditions. Examples of soil amendments include, for example, alterations in microorganism community structure, temperature, pH, dissolved oxygen concentration, salt concentration, macro nutrient levels, micro nutrient levels and the like.

The ∝ subunits of aromatic oxygenase genes are targeted in preferred embodiments of the invention because they have been implicated in substrate specificity, and DNA sequences encoding oxygenases targeting the same substrate have been found to include conserved regions. Oxygenases play a key role in aerobic metabolism by hydroxylating the ring or side chains of aromatic hydrocarbons. Dioxygenases initiate biodegradation of benzene, toluene, naphthalene, biphenyl, and other aromatics by incorporation of both atoms of molecular oxygen into the ring. Benzene, toluene, phenol, and xylenes are also attacked by monooxygenases which incorporate a single atom of molecular oxygen into the ring or side groups of aromatic compounds. Many of the aromatic oxygenases are multicomponent enzyme complexes composed of a terminal oxygenase (α and β subunits), a ferredoxin, and a ferredoxin reductase. Although some debate remains regarding the roles of the α and β subunits in substrate specificity, research has shown that the α subunit is at least partially responsible for substrate specificity of biphenyl and toluene dioxygenases.

DNA sequences encoding some important aromatic oxygenases have been determined and published. To quantify the bioremediation potential of a sample for a given aromatic pollutant in accordance with the invention, the first step is to make, select, or otherwise provide a PCR primer set or a series of PCR primer sets that are effective to amplify a polynucleotide target region that is conserved, or that includes sub-regions that are conserved, among diverse microbial species that have known oxygenase functionality for the selected pollutant. In one preferred manner of making a primer set in accordance with the invention, a sequence alignment of genes is prepared to include genes from diverse species that encode oxygenase enzymes effective to oxygenate the selected pollutant. Using the alignment, a region is identified, such as, for example, a region of from about 50 to about 1000 bases in length, that is substantially conserved in a plurality of genes in the alignment or that includes two or more sub-regions that are substantially conserved in a plurality of genes in the alignment. As used herein, the term “substantially conserved” is used to refer to a degree of homology sufficient to effect hybridization to a single primer or probe under hybridization conditions of the selected Q-PCR protocol. A primer set can then be made by preparing a forward primer of from about 10 to about 40 bases complementary to a nucleotide segment of one strand of the region and a preparing a reverse primer of from about 10 to about 40 bases complementary to a nucleotide segment of the complementary strand of the region. The forward and reverse primers can be prepared to span a target region comprising all or a portion of a conserved region or can be prepared to anneal to two separate conserved sub-regions of suitable proximity that span a non-conserved segment of some or all of the genes in the alignment. The primer set is effective to amplify template strands corresponding to the target region from a plurality of genes comprising the conserved region or sub-regions by quantitative PCR when such genes are present in the sample.

Alignments constructed of the ∝ subunits of multiple oxygenase genes, discussed further in the Examples below, reveal families or subfamilies having conserved regions based on target pollutant compound. For example, these alignments reveal that the toluene dioxygenase is more closely related genetically to other toluene and benzene dioxygenases than to biphenyl dioxygenase genes. Close examination of these alignments reveals conserved regions that are unique for each family or subfamily of aromatic oxygenase genes. For example, the a subunits of toluene dioxygenases share a greater sequence identity to each other than to even the closely related biphenyl dioxygenase subunits. Regardless of the mechanism causing the divergence of α subunits that led to different families or subfamilies of oxygenases based on substrate specificity, two generalizations can be made: (1) Each aromatic oxygenase family or subfamily can initiate biodegradation of an environmentally important aromatic hydrocarbon and (2) Different families or subfamilies of oxygenases can be distinguished at the DNA level. The present invention provides methods for detecting and distinguishing aromatic oxygenase families and subfamilies.

It is also important to ensure that primers made or selected in accordance with the invention do not amplify sample components that are not within the genotype being targeted. Alignments that have been prepared for aromatic oxygenases described herein have been designed to include related subfamilies to ensure that conserved regions are unique to a given subfamily that the primers are designed to amplify and detect. Primers specifically identified herein have also been submitted to GenBank and compared with known sequences in the database as a further effort to ensure that the sequences selected for use as primers are unique to the targeted genes. The use of conserved DNA sequences for primers is expected to allow detection of a wide variety of genes of a given genotype from a diverse population of microbe species in the environment while still meeting the need for substrate specificity, and thus biodegradation functionality. Thus, PCR amplification of a fragment of an aromatic oxygenase gene using subfamily specific primers based on a consensus sequence allows detection of a wide variety of related pathways. The present invention provides methods for making PCR primers based on such conserved regions or sub-regions, which allows selective amplification of targeted aromatic oxygenase genes, i.e., genes encoding oxygenases having specified functionality, irrespective of the identity of the microbe species from which the gene is isolated.

If, during the course of identifying conserved regions or sub-regions, the identified conserved region or sub-region is present in each gene represented in the alignment, quantitative PCR analysis using the primer set is expected to effectively and selectively detect and quantify the genotype. If, however, genes are present in the alignment that have the stated functionality but do not include the conserved region or sub-regions, the effectiveness of the quantitative PCR analysis can be increased by identifying a second region, such as a region of from about 50 to about 1000 bases in length, that is substantially conserved, or that includes two or more sub-regions that are substantially conserved, in one or more additional genes in the alignment and that is not conserved in the genes targeted by the first primer set or in other genes expected to be potentially present in the sample. If a second region is identified as described, a second primer set can be prepared that is effective to amplify template strands in the sample corresponding to a second target region spanning all or a portion of the second conserved region or spanning a non-conserved segment between two conserved sub-regions. Since the second primer set does not target the genes targeted by the first primer set, quantification by quantitative PCR using both primer sets will not result in duplicate detection of a gene, which could result in loss of accuracy.

If the first region or the second region is present in each gene represented in the alignment, quantitative PCR analysis using both primer sets is expected to effectively detect and quantify all or substantially all representatives of the microbial community in the sample that are functional to oxygenate the selected aromatic pollutant. If there are genes in the alignment that are not targeted by the first or second primer set, additional conserved regions can be sought as described above. Alternatively, if other gene sequences in the alignment are not targeted by the first or second primer set, it is possible to prepare additional primer sets that target the remaining genes specifically to improve the correlation of the PCR results to the bioremediation potential of microbes in the sample for the specified pollutant. It is also possible to proceed with PCR quantification using primer sets that do not amplify all of the genes in the alignment. Such quantification would also be expected to correlate to the quantity of genotypes present in the sample, which information would be useful for assessing the bioremediation potential of the sample.

It is, of course, understood that the above procedure can be used to prepare primer sets for detecting and quantifying genotypes that metabolize a wide variety of aromatic pollutants for which it is desired to determine bioremediation potential. Indeed, as described further in the Examples, the present inventors have identified multiple exemplary primer sets in accordance with the invention that are effective to amplify target regions of multiple genotypes encoding oxygenase enzymes having specificities for multiple diverse aromatic pollutants. These exemplary primer sets are described further below; however, it is not intended that the invention be limited to these primer sets, it being understood that the processes for making primer sets described herein can be used to construct alternative primer sets in accordance with the invention. In addition, the present invention also specifically contemplates variants of the exemplary primers described herein that also suitably target the desired conserved region by virtue of their homology to the primer sequences set forth herein. In this regard, the present invention contemplates primers comprising at least about 10 consecutive nucleotides of the sequences set forth as SEQ ID NOs: 1-19, and primers that have at least 80% identity to the sequences set forth as SEQ ID NOs: 1-19, or portions thereof, and that are effective to amplify the respective target sequences. In other embodiments, primers are provided that have at least 90% identity to the sequences set forth as SEQ ID NOs: 1-19. In yet other embodiments, primers are provided that have at least 95% identity to the sequences set forth as SEQ ID NOs: 1-19.

Percent identity may be determined, for example, by comparing sequence information using the MacVector computer program, version 6.0.1, available from Oxford Molecular Group, Inc. (Beaverton, Oreg.). Briefly, the MacVector program defines identity as the number of identical aligned symbols (i.e., nucleotides or amino acids), divided by the total number of symbols in the shorter of the two sequences. The program may be used to determine percent identity over the entire length of the polynucleotide being compared or over a portion thereof. Preferred default parameters include: (1) for pairwise alignment parameters: (a) Ktuple=1; (b) Gap penalty=1; (c) Window size=4; and (2) for multiple alignment parameters: (a) Open gap penalty=10; (b) Extended gap penalty=5; (c) Delay divergent=40%; and (d) transitions=weighted. The invention also contemplates a primer having a sequence sufficiently similar to those set forth herein to hybridize thereto under conditions suitable for a PCR reaction.

In another aspect of the invention, there are provided methods for assessing the bioremediation potential of a microbial community in a soil or water sample. Primers constructed as described above can be advantageously used in quantitative PCR protocols to quantify genotypes in environmental samples. Calculated gene copy numbers can then be used to evaluate biodegradation potential of a given sample for specific pollutants.

In one manner of assessing the bioremediation potential of microbes in a sample in accordance with the invention, a plurality of PCR primer sets are provided that correspond to distinct families or subfamilies of functional aromatic oxygenase genes, and that are effective to selectively amplify target regions from diverse aromatic oxygenase genes in the corresponding family or subfamily. A mixture of polynucleotides are extracted from microbes present in a soil or water sample or otherwise provided after extraction from microbes present in a sample. Quantitative PCR analysis of the mixture is then performed using the PCR primer set to quantify the bioremediation potential of microbes in the sample. Q-PCR analysis can advantageously include one or more Q-PCR amplification reactions using the primer sets to quantify diverse aromatic oxygenase genes of each corresponding family or subfamily in the mixture. Bioremediation potential of microbes in the sample can then be assessed based upon results of the one or more Q-PCR reactions.

Microorganism-containing samples that can be analyzed in accordance with the invention can be collected from a natural environment or artificial environment. Examples of natural environmental samples include sea water, lake water, river water, bottom mud, sediment, soil, minerals, underground water, pore water, and plants and animals. Examples of artificial environmental samples include, for example, samples prepared in a laboratory to impose certain conditions upon a microbial community therein. Microorganisms in a sample can be concentrated by means such as filtration and centrifugation when there are few microorganisms in these environments. For example, the microorganisms in environmental water can be concentrated on a filter by filtration using a filter such as a membrane filter or hollow-fiber membrane filter with a pore size of 0.2 μm, which is smaller than the cell size of many common microorganisms. The product of this procedure can be used as the sample. Alternatively, sample water can be filtered by passing it horizontally rather than vertically using for example a tangential flow filter (Millipore, Bedford, Mass.) with a membrane filter with a pore size of 0.2 μm, and the resulting concentrated solution can be used as the sample. The microorganisms can also be precipitated and concentrated by subjecting the sample directly to high-speed centrifugation, e.g., by centrifuging for 10-100 min at approximately 8000×g or more, and the resultant sample can also be used for nucleic acid extraction.

Known methods can be used for extracting polynucleotides, i.e., DNA or RNA, from a microorganism-containing sample. Purification techniques using hydroxyapatite are advantageous in the case of samples such as soil and sediment. When the subject of analysis is RNA, a commercially available RNA extraction kit such as a Qiagen RNEASY KIT™, Stratagene RNA RT-PCR Miniprep kit, Clontech NUCLEOSPIN™ RNA kit, or Ambion RNAQUEOUS™ kit may be used. When the sample contains a large amount of contaminants, the efficiency of purification of the extracted polynucleotide mixture can be improved by combining several of these nucleic acid extraction methods. The degree of purification can be confirmed easily by measuring the spectrum of absorbance near a wavelength of 220 to 400 nm by spectrophotometer and comparing it with that of pure RNA and DNA samples. In all cases, careful attention should be given to preventing contamination of biological materials such as DNase or RNase during the extraction procedure.

Although the final amplicon concentration after PCR amplification is not proportional to starting template concentration, variations of the basic PCR protocol have been developed to permit quantification. The quantitative PCR analysis used in accordance with the invention can be a competitive PCR protocol or, more preferably, a real-time PCR protocol. In competitive PCR, a comparison of the intensities of a standard and the target amplicons allows quantification. More particularly, competitive PCR relies on spiking reactions with a serial dilution of a known amount of internal standard that shares the same primer recognition and internal sequences as the target. Ideally, the target and competitor are substantially identical except for a minor difference in size and are therefore amplified with the same efficiency. As the name implies, the target and competitor then compete for reagents based on their relative concentrations. A comparison of target and competitor intensities on a gel after completion of PCR yields quantification. When amplification efficiencies truly are equivalent, competitive Q-PCR can be an accurate method of quantification. Although accurate and sensitive, competitor design and post-amplification processing is labor intensive making competitive Q-PCR somewhat less attractive for large-scale processing of environmental samples. Another limitation of this method is that amplification efficiency of the target and standard are not always equivalent, which decreases the accuracy of the results.

The addition of fluorescence techniques and detection to PCR has led to real-time or kinetic Q-PCR in which product formation is monitored during the course of the reaction. Real-time PCR offers an accurate, sensitive method of quantification without the labor-intensive post-amplification analysis and assumptions required by competitive PCR. Real-time PCR utilizes reagents that generate a fluorescence signal proportional to the number of amplicons produced by the PCR process. Real-time PCR is based upon the principle that, the more template initially present, the fewer number of cycles are necessary to reach exponential phase where the fluorescence signal rises above the background signal. This point, called the threshold cycle, occurs during the exponential phase and is proportional to the initial template concentration. Thus a standard curve can be generated with gene copy numbers as a function of the threshold cycle to permit quantification of unknown samples without any post-amplification sample processing.

The differences in various real-time PCR protocols rests in methods for generating a fluorescence signal with the amplification product. The simplest method is to add to each reaction a DNA-binding dye, such as, for example, SYBR Green, that fluoresces only upon binding to double stranded-DNA (“ds-DNA”), and measure fluorescence as an increasing quantity of dye is bound to the ds-DNA during the polymerization step in each cycle. This method, which can be referred to as an intercalator-based method, has been reported to quantify as low as 10 copies per reaction, however, problems often arise with background fluorescence levels. Other real-time PCR methods, commonly referred to as probe-based methods, use molecular beacons, hybridization probes, and hydrolysis probes, and rely on hybridization of an internal probe during PCR to produce a fluorescence signal. Molecular beacons are hybridization probes with fluorescent markers and quenchers on opposite ends of the probe. In solution, molecular beacons form stem-and loop-structures holding the marker near the quenchers, but upon hybridization, the separation of the marker and quencher allows fluorescence. The main disadvantage of this method is that the probe and target must match exactly because the thermodynamic properties of the beacon favor the hairpin structure. The hybridization probe method employs two hybridization probes—one with a fluorescein donor on its 3′ end and the other with an acceptor fluorophore at its 5′ end. Upon hybridization the probes anneal in a head-to-tail arrangement to bring the donor and acceptor into close proximity permitting a signal. The hydrolysis probe (Taqman) assay takes advantage of the 5′-nuclease ability of DNA polymerase to hydrolyze a labeled probe bound to its target amplicon to produce a signal. Although the hydrolysis probe methods offer an additional degree of specificity, this advantage must be weighed against the increased complexity of designing the assay. Furthermore, the probe as well as the primers must be from a highly conserved region of the target to avoid biasing quantitation.

Assessing bioremediation potential using quantitative PCR in accordance with the invention can utilize any type of quantitative PCR, whether of the competitive or real-time variety; however, in one particularly preferred embodiment of the invention, quantitative PCR is achieved using a real-time PCR protocol and a ds-DNA binding dye, such as, for example SYBR Green. PCR protocols used in connection with the invention also preferably utilize a Hotstart polymerase, which results in a reduction in background fluorescence signal compared to other commercially available polymerases.

In one particularly preferred manner of practicing the invention, the polymerization temperature selected for use in the PCR protocol is a temperature of from about 3 to about 10° C. below the melting temperature of the desired products of the amplification reaction. A person of ordinary skill in the art will appreciate that the melting temperature of the desired products is a temperature above which the reaction product will dissociate. In another preferred embodiment, the polymerization temperature is a temperature of from about 4 to about 8° C. below the melting temperature of the desired products. In another preferred embodiment, the polymerization temperature is a temperature of about 5° C. below the melting temperature of the desired products. Optimization of the polymerization temperature in this manner has been found to significantly decrease background fluorescence signals, and decreases detection limits of the protocol. A detection limit of 10³ copies/reaction (10⁴ copies/g soil) was achieved with the exemplary primers described herein and the SYBR Green method, which is considered to be very adequate for site remediation assessment purposes.

The invention therefore also provides in one aspect a method for determining a polymerization temperature for a PCR reaction that includes determining the melting temperature of the one or more desired products of the amplification reaction, and setting the polymerization temperature for the protocol at a temperature of from about 3 to about 10° C., more preferably about 4 to about 8° C., and still more preferably about 5° C. below the melting point. It is understood that the melting temperature of a given amplification product is affected by the length of the amplicon, the nucleotide content of the amplicon and other factors. Thus, in addition to determining the melting temperature for a given product, it is also possible to alter the amplicon length or content by selecting primers effective to amplify target regions of differing lengths or having different nucleotide compositions within a given conserved region. This is particularly useful in embodiments of the invention in which multiplex PCR amplification is used, as discussed further below. A multiplex amplification reaction is most effective when the primer sets used together in the reaction have annealing temperatures that are relatively close. Furthermore, detection limit of the reaction can be optimized by selecting or designing primers that produce amplicons having melting temperatures that are also relatively close, so that a polymerization temperature can be selected to decrease the detection limits of the protocol.

In another embodiment of the invention, multiple primer sets targeting diverse aromatic pollutant degrading genotypes, i.e., targeting diverse families or subfamilies of functional aromatic oxygenase genes, can be used together in a multiplex real-time PCR protocol, which further reduces the time required to assess bioremediation potential of microbes in a given sample. In this regard, the term “multiplex real-time PCR” refers to a PCR protocol in which primer sets targeting diverse families or subfamilies of functional aromatic oxygenase genes are mixed together in a single reaction mixture to detect and quantify diverse genotypes in a single amplification run. In certain multiplex PCR protocols, the diverse genotypes can be separately detected and quantified by including probes in the amplification mixture that selectively target the various amplification templates (i.e., the diverse genotypes), and that are labeled with fluorescing groups that fluoresce at different wavelengths. Using such probes, the diverse genotypes are separately detected and quantified by measuring the fluorescence of the fluorescing groups at the different wavelengths.

The present invention also provides a novel approach for real-time PCR in which a non-selective ds-DNA dye is used in a multiplex PCR reaction. In this regard, the present invention contemplates multiple scenarios in which it is desirable to detect and quantify the total presence of multiple genotypes in a sample, but it is not necessary to determine the relative proportion of the genotypes in the sample, and thus determining the proportional quantification of each of the various genotypes is not necessary. In such a scenario, the advantages of multiplex real-time PCR can be utilized while also utilizing the advanteges of a ds-DNA binding dye, and eliminating the need for developing diverse probes with diverse fluorescence characteristics. In such a scenario, primer sets targeting diverse genotypes can be used in a multiplex real-time PCR with a single ds-DNA binding dye such as, for example, SYBR Green, to provide a sum quantification of the genotypes present in the sample that are targeted by the selected primers.

The multiplex real-time PCR protocol described above can be used, for example, when multiple primer sets are used to detect a family or subfamily of genes that encode oxygenases having specificity for a single aromatic pollutant. Alternatively, it may be suitable when assessing bioremediation potential of a given sample polluted with multiple aromatic pollutants to simply score the overall bioremediation potential of the sample rather than the specific bioremediation potentials of the sample for metabolizing each of the individual pollutants. Indeed, such multiplex real-time PCR protocols can be used to advantage in connection with other scoring methods, if desired, to provide more information regarding the bioremediation potential of a sample as it relates to one or more of the specific pollutants present in the sample. In this regard, a mulitiplex PCR protocol as described above can be used in conjunction with one or more single-plex PCR protocols to assess the proportion of the muliplex PCR quantification signal that is attributable to a given genotype in the sample. Alternatively, amplified products from the multiplex PCR protocol can be further analyzed, for example, using standard electrophoresis procedures, to determine the lengths of amplicons in the multiplex PCR amplification product, which will provide further information regarding the genotypes present in the sample.

It is of course important to recognize that primer sets can be effectively used together in a multiplex PCR protocol only if the primer sets have annealing temperatures that are sufficiently similar. In this regard, multiple primer sets described herein have been found to have annealing temperatures that are sufficiently close that the primer sets can be advantageously used together in a multiplex real-time PCR protocol as described herein. For example, the work reported in the Examples below demonstrates that the PHE and NAH primer sets are suitable for use in a multiplex PCR protocol, as are the TOL and TOD primer sets and the BPH2 and BPH4 primer sets.

The present invention also contemplates the use of inventive principles in reverse transcriptase PCR (RT-PCR) protocols. In this regard, PCR amplification with a DNA target sequence is useful for assessing bioremediation potential; however, RT-PCR using mRNA isolated from a soil or water sample as the template is effective to identify and quantify the genotypes that are actively being expressed in a given sample, and is therefore effective for directly assessing point-in-time degradation activity. In addition to, or as an alternative to, assessing gene expression at contaminated sites, an RT-PCR protocol can be used in certain embodiments of the invention, with primers provided in accordance with the invention, as a point-in-time bioremediation assay. RT-PCR protocols can also be used in accordance with the invention to study the effects of co-occurring substrates on pathway regulation. For example, detection of naphthalene and biphenyl dioxygenase genes at the gasoline-contaminated sites reported in the Examples suggests that they may play a role in BTEX biodegradation. In the microcosm study reported in the Examples, putative naphthalene dioxygenase genes were detected in the benzene and o-xylene microcosms. RT-PCR amplification of mRNA extracts of these samples would indicate if naphthalene dioxygenase was actively expressed in response to these pollutants.

Enumeration of aromatic oxygenase gene expression with a real-time RT-PCR protocol also provides a direct indicator of the effect of site perturbations on the functional activity of the microbial population. This information can be coupled with chemical data from flux meters to (1) document biodegradation at monitored natural attenuation sites, (2) optimize oxygen and nutrient additions at engineered remediation sites, and (3) assess the effect of co-occurring technologies like surfactant flushing on biodegradation of aromatic compounds.

In another aspect, the present invention provides multiple exemplary primer sets that have been constructed for use in a quantitative PCR protocol to assess the bioremediation potential of a sample vis-à-vis a wide variety of possible pollutants. In this regard, PCR primers targeting the ∝ subunits of phenol hydroxylase, toluene monooxygenase, toluene dioxygenase, toluene ring-hydroxylating monooxygenases, naphthalene dioxygenase, and biphenyl dioxygenase are provided. These primers, coupled with quantitative PCR can be used to detect and quantify copy numbers of a wide variety of important aromatic oxygenase genes. Because oxygenase enzymes mediate the initial oxidation of a variety of aromatic hydrocarbons, the compositions and protocols provided by the present invention, in particular when used in connection with real-time PCR protocols, allow rapid screening of environmental samples for known aromatic catabolic pathways, thus allowing evaluation of the feasibility of bioremediation as a treatment technology. Exemplary primer sets provided by the invention are set forth in Table 3. A series of PCR-based assays using primers made or selected in accordance with the invention can advantageously be used in a large-scale, high throughput manner to detect and enumerate catabolic genes involved specifically in the biodegradation of aromatic pollutants.

In another aspect of the invention, there is provided an excellent petroleum catabolic screen that includes a combination of PHE/NAH multiplex PCR, TOL/TOD multiplex PCR, and PCR with the RDEG primers. In another embodiment, a biphenyl dioxygenase screen consisting of PCR with BPH1 primers and BPH2/BPH4 multiplex PCR is useful to determine the presence of known biphenyl dioxygenase genes. Considering biphenyl dioxygenase genes and ring-hydroxylating monooxygenase genes were detected in P. aeruginosa JI104, a biphenyl dioxygenase screening would be valuable for petroleum contaminated sites in addition to sites in which PCBs are encountered. Of course, protocols using other combinations of primers are also contemplated and, indeed, may be more desirable in contamination sites containing different combinations of aromatic pollutants. The multiplex PCR protocols, when used, advantageously reduce the number of runs required and therefore decrease time needed to screen environmental samples.

In yet another aspect of the invention, there is provided a kit of reagents for performing a real-time PCR-type amplification reaction for detecting and quantifying aromatic oxygenase genes in a sample. In one embodiment, the kit includes a plurality of primer sets as provided herein that target different families or subfamilies of aromatic oxygenase genes. In another embodiment, a kit is provided that also includes a ds-DNA binding dye. In yet another embodiment, a kit is provided for performing competitive Q-PCR. The kit includes a plurality of primers made or selected in accordance with the invention and also includes standards for use in the Q-PCR protocol. In an alternative embodiment, a kit is provided for performing probe-based real-time PCR that includes a plurality of primers in accordance with the invention and also includes a plurality of probes effective to hybridize to polynucleotides targeted by the primers under annealing conditions of the PCR protocol.

In addition to field applications, the primers and quantitative PCR protocols described herein provide a direct and accurate means of addressing remaining questions regarding the biodegradation of aromatic hydrocarbons. Although successful amplification from environmental samples has been cited indicating that known aromatic catabolic pathways may play a role in the field, little quantitative evidence has been given. Real-time PCR with oxygenase specific primers in accordance with the invention provides an opportunity to quickly and accurately investigate the microbial ecology of petroleum-contaminated sites to determine the role of currently characterized pathways. While contaminated sites have been estimated to contain 100 to 200 distinct aromatic hydrocarbons, most investigations have focused on biodegradation of pure compounds by cultured strains. Use of the present invention provides scientists and engineers with direct and more accurate feedback on the effectiveness of operating variables (e.g. oxygen addition) than culture-based assays and would compliment contaminant removal data for site characterization. Furthermore, substrate interactions including competitive inhibition have been noted with mixtures of aromatic substrates. The PCR primers described here, when used with real-time RT-PCR, allow rapid quantification of the effect of mixtures of substrates and provide insight into biodegradation in the field.

Although the invention is described herein in terms of detecting genotypes relating to aromatic oxygenases, inventive principles can also be used in other applications in which it is desirable to specifically identify whether a genotype is present without needing to determine the exact identification of a species. Thus, inventive methods for making primers is considered to be equally applicable to other microbial systems in which the quantification of a genotype is desired, and in which microbes having similar functionality have conserved regions that correspond to functionality. One example of such a system is a waste water treatment system, thought other systems featuring dynamic microbial degradation are also contemplated.

Reference will now be made to specific examples illustrating various preferred embodiments of the invention as described above. It is to be understood, however, that the examples are provided to illustrate preferred embodiments and that no limitation to the scope of the invention is intended thereby.

### EXAMPLES

The experimental work reported herein relates to the development of quantitative polymerase chain reaction (PCR) procedures, including multiplex and real-time PCR procedures, and the development of primers for use in said procedures, to quantify aromatic catabolic genes that were then used to investigate the selection of aromatic catabolic pathways in laboratory microcosms and environmental samples from petroleum-contaminated sites. The inventive procedures and primers are useful in the assessment of bioremediation potential of a polluted site.

Aromatic oxygenases were chosen as a preferred type of indicator genes for bioremediation potential because they mediate the first and rate-limiting step in aromatic hydrocarbon biodegradation and their DNA sequences are conserved within families of oxygenase genes. PCR primer sets were chosen from conserved regions unique to each family of oxygenases observed during alignments of known gene sequences. Thus each primer set is specific for a family of oxygenase genes (e.g. toluene dioxygenase) without excluding closely related but uncharacterized oxygenase genes. In all, primer sets were identified which allowed amplification of an initial oxygenase gene from pathways for the catabolism of naphthalene, biphenyl, benzene, toluene, xylenes, and phenol.

With positive control strains, the length of the observed amplification product matched that predicted from published sequences and specificity was confirmed by hybridization for all primer sets. Optimization of polymerization temperatures for real-time PCR greatly reduced background fluorescence signals allowing detection limits of 10³ gene copies per reaction. Following development of PCR assays, laboratory microcosms with single aromatic substrates (enrichment substrates) were prepared to test the PCR assay with uncharacterized bacteria and evaluate the selective pressure exerted on the soil microbial community by aromatic hydrocarbon contamination. For each microcosm, at least one family of oxygenase genes responsible for the biodegradation of the enrichment substrate was amplified using the primers developed. Results from the microcosm study gave insight into the selection of aromatic catabolic pathways in the environment and indicated that primers were specific for their targets in a complex pool of unknown DNA. Finally, groundwater samples from two gasoline-contaminated sites were studied. In field samples, aromatic oxygenase genes were detected in groundwater monitoring wells with current or recent petroleum contamination but not in wells with no history of contamination, confirming that this technology is appropriate for monitoring pollutant biodegradation.

### Example One

This study was conducted to develop multiplex and real-time PCR procedures to quantify aromatic catabolic genes in environmental samples. The large subunit of aromatic oxygenase genes was chosen as the indicator gene because it has been implicated in substrate specificity, is one of the rate-limiting steps in aromatic hydrocarbon biodegradation, and its DNA sequence is conserved for oxygenases targeting the same substrate. Alignments were constructed from groups of related oxygenase genes and each primer set was chosen from a conserved region unique to that group of oxygenases. Thus a single primer set will detect an entire subfamily of related oxygenase genes rather than a species-specific catabolic gene. In all, PCR primer sets were identified which targeted biphenyl dioxygenase, naphthalene dioxygenase, toluene dioxygenase, toluene/xylene monooxygenase, phenol monooxygenase, and ring hydroxylating-toluene monooxygenase genes. Testing and optimization with genetically well-characterized bacterial strains demonstrated the specificity of each primer set. Multiplex PCR protocols were developed to permit simultaneous detection of aromatic oxygenase genes and facilitate rapid screening of environmental samples. Real-time PCR with SYBR green I was used to quantify gene copy number with a quantification limit of 10³ copies of target per reaction. The primer sets and real-time PCR methods presented are useful for assessing natural attenuation, for investigating contaminated site-ecology, and for aiding in optimization of bioremediation in the field.

Materials and Methods

Bacterial Strains and Growth Conditions

Liquid cultures were grown overnight in minimal medium containing (per liter) 2 g NH₄Cl, 1 g NaH₂PO₄×H₂0, 4.25 g K₂HPO₄×3H₂0, 0.001 g ZnSO₄×7H₂), 0.001 g MnSO₄×H₂0, 0.003 g FeSO₄×7H₂O, and 0.025 g MgSO₄ supplemented with the appropriate carbon source with shaking (125 rpm) at 30° C. (Mesarch, M. B. and L. Nies. 1997. Modification of heterotrophic plate counts for assessing the bioremediation potential of petroleum-contaminated soil. Environmental Technology 18:639-646.). Biphenyl and naphthalene were added as solids to the liquid medium or to the lids of inverted agar plates. Toluene was provided as a gas by allowing 1 ml volume of toluene to volitalize from an autosampler vial with a pierced septa in sealed containers (Ridgway, H. F., J. Safarik, D. Phipps, P. Carl, and D. Clark. 1990. Identification and catabolic activity of well-derived gasoline degrading bacteria from a contaminated aquifer. Applied and Environmental Microbiology 56:3565-3575.). Rhodococcus sp. RHA1, Rhodococcus erythropolis TA421, Pseudomonas aeruginosa JI104, and Pseudomonas mendocina KR1 were grown on C-medium (Maeda, M., S.-Y. Chung, E. Song, and T. Kudo. 1995. Multiple genes encoding 2,3-dihydroxybiphenyl 1,2-dioxygenase in the gram positive polychlorinated biphenyl-degrading bacterium Rhodococcus erythropolis TA421, isolated from a termite ecosystem. Applied and Environmental Microbiology 61:549-555.).

DNA Extractions

DNA extractions followed the protocol as described by Marmur (Marmur, J. 1961. A procedure for the isolation of deoxyribonucleic acid from micro-organisms. Journal of Molecular Biology 3:209-218.). Approximately 1.5 ml of cell cultures were incubated with 2 mg/L lysozyme at 37° C. for an hour and then centrifuged at 14,000×g for two minutes to obtain a cell pellet. This pellet was then resuspended in 567 μL Tris-EDTA buffer (TE—10 mM Tris-Cl, 1 mM EDTA, pH8.0), 30 μL of 10% sodium dodecylsulfate (SDS), and 3 μL of proteinase K. This mixture was incubated at 37° C. for an hour to lyse cells and denature proteins. Following the lysis incubation, 100 μL of 5M NaCl was added and the solution was mixed. Next, 80 μL of a hexadecyltrimethyl ammonium bromide solution (10 CTAB in 0.7M NaCl) was added, the solution was mixed and incubated at 65° C. for 10 minutes. An equal volume (750 μL) of chloroform/isoamyl alcohol (24: 1) was added to the lysed cells. The mixture was then mixed and centrifuged for 5 minutes at 14,000 rpm (approximately 19,000×g). The aqueous (upper) layer containing the DNA extract was then transferred to a fresh microcentrifuge tube. To this fraction, an equal volume of phenol/choloroform/isoamyl alcohol (25:24:1) was added, mixed, and the solution was centrifuged for 5 minutes. Again the aqueous phase was transferred to a fresh tube. Approximately 0.6 volumes (450 μL) of cold isopropanol was slowly added to the aqueous phase and the solution was mixed gently by inversion until the DNA precipitated. The DNA was then centrifuged (5 minutes at 14,000 rpm), washed with 1 ml of 70% ethanol, and centrifuged again. The wash ethanol was discarded and the DNA pellet was allowed to air dry for approximately 15 minutes. Purified DNA was then resuspended in 100 μL of TE.

DNA extractions from some pure cultures were performed using the FastDNA kit (BIO101, Vista, Calif.) and the FP120 FastPrep Cell Disruptor (Savant Instruments Inc., Holbrook, N.Y.) as per instructions provided. Briefly, 200 μL of cell cultures were added to tubes containing 1 ml of CLS-TC lysis solution and a 0.25 inch sphere designed to lyse cells by mechanical disruption. The tubes were then placed in the FP120 FastPrep Cell Disruptor and homogenized to lyse cells and release DNA into solution. The heat generated by mechanical disruption deformed the tubes so they were placed on ice for 5 minutes. The tubes were then centrifuged for 5 minutes at 14,000×g to pellet cellular debris. Then 600 μL of the supernatant was transferred to a fresh microocentrifuge tube containing 600 μL of Binding Matrix. The mixture was incubated at room temperature for five minutes to allow the dissolved DNA to bind to the matrix. Then the tube was centrifuged for one minute at 14,000×g and the supernatant was discarded. The Binding Matrix pellet was then washed gently with 500 μL of NewWash solution and centrifuged as before. Next, the DNA was eluted from the Binding Matrix by resuspending in 100 μL of TE. Following 2 to 3 minutes of incubation, the tube was centrifuged for one minute at 14,000×g to pellet the Binding Matrix. The supernatant containing purified DNA in TE was transferred to a fresh tube for storage.

DNA Quantification

DNA concentrations were quantified by fluorometry using a Model TKO100 DNA Fluorometer (Hoefer Scientific Instruments, San Francisco, Calif.) The fluorometer was calibrated with 100 ng μl⁻¹ calf thymus DNA by adding 2 μL of the standard solution to 2 ml of assay solution. The assay solution contained (per 100 ml) 10 ml of 10×TNE buffer, 90 ml of distilled water, and 10 μL of Hoechst 33258 dye (bisbenzimide). The dye binds to the DNA in the sample and fluoresces when excited by light at 365 nm. Having calibrated the instrument, unknown DNA samples were quantified by adding 2 μL to 2 ml of assay solution in a glass cuvette and the result was read from the fluorometer in ng μL⁻¹.

Agarose Gel Electrophoresis

Aliquots of DNA extracts were visualized on 0.7% agarose gels (Bio-Rad, Richmond, Calif.) in 1× Tris-Acetate-EDTA (TAE) buffer stained with ethidium bromide (0.0001%). In addition to 5 μL of DNA extract, 1 μL of loading dye and 4 μL of TE were loaded into each well. The loading dye, containing 0.25% bromophenol blue, 0.25% xylene cyanol FF, and 15% ficoll in water (Sambrook et al., 1989) was added to increase the density of the aliquot and prevent the sample from floating out of the well. Following separation by electrophoresis, gels were visualized under ultraviolet light and photographed as needed. Visualizing DNA extracts on a gel provided an initial estimation of concentration but more importantly allowed evaluation of the quality of the extraction.

In all cases, 10 μL of PCR products were visualized on 1% agarose gels also stained with ethidium bromide. A 100- to 3000-bp marker was also run along side PCR products to give an indication of fragment size.

Phylograms and Alignments

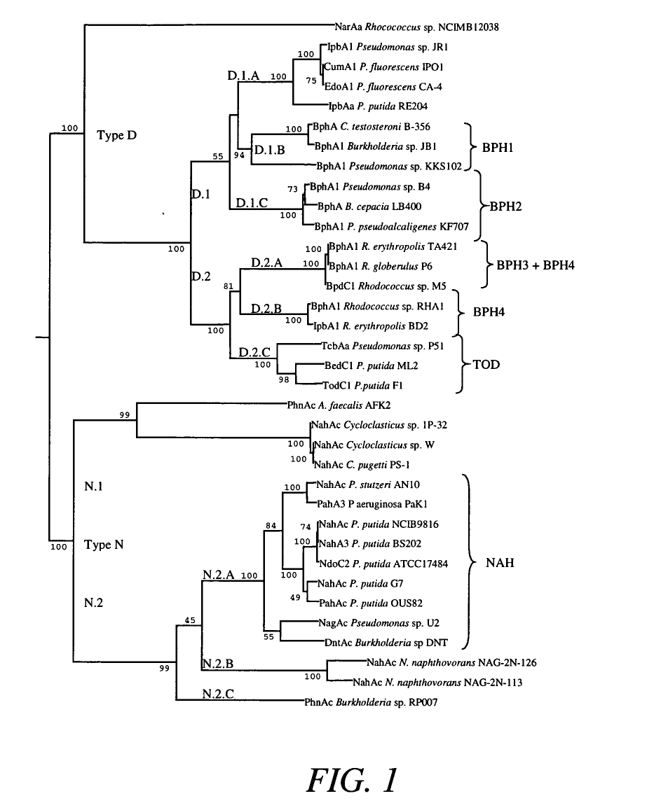

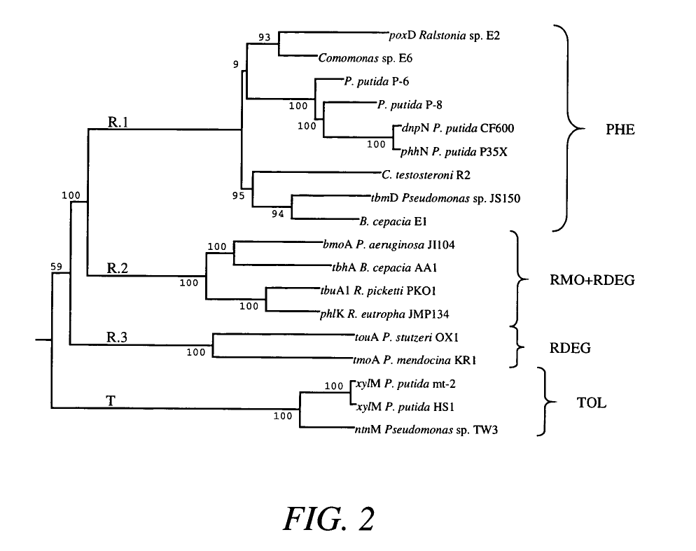

DNA and amino acid sequences of the large subunits of aromatic oxygenases were retrieved from GenBank and aligned using ClustalW 1.7 (Thompson, J. D., D. G. Higgins, and T. J. Gibson. 1994. CLUSTAL W: improving the sensitivity of progressive multiple sequence alignment through sequence weighting, positions-specific gap penalties and weight matrix choice. Nucleic Acids Research 22:4673-4680.). Genes used for these alignments are given in Tables 1 and 2. Phylograms were constructed with DNAMAN software programs (Lynnon BioSoft, Vaudreuil, Quebec, Canada) using the Neighbor-Joining method (Saitou, N. and M. Nei. 1987. The neighbor-joining method: a new method for reconstructing phylogenetic trees. Molecular Biology and Ecology 4:406-425.) and bootstrapping analysis. The phylograms are set forth in FIGS. 1 and 2.

TABLE 1
Aromatic Dioxygenase Genes used to Deduce Conserved
Regions for PCR Primers.

| | Accession | |
|---|---|---|
| Gene | Number | Source Organism |
| Biphenyl | | |
| dioxygenase | | |
| bphA | U47637 | Comanonas testosteroni B-356 |
| bphA1 | D17319 | Pseudomonas sp. KKS102 |
| bphA1 | X80041 | Rhodococcus globerulus P6 |
| bpdC1 | U27591 | Rhodococcus sp. M5 |
| bphA1 | D88021 | Rhodococcus erythropolis TA421 |
| bphA | M86348 | Burkholderia sp. LB400 |
| bphA1 | M83673 | Pseudomonas pseudoalcaligenes KF707 |
| bphA1 | D32142 | Rhodococcus sp. RHA1 |
| bphA1 | AJ010057 | Burkholderia sp. JB1 |
| bphA1 | U95054 | Pseudomonas sp. B4 |
| Isopropylbenzene | | |
| and ethylbenzene | | |
| dioxygenases | | |
| ipbA1 | U24277 | Rhodococcus erythropolis BD2 |
| ipbA1 | U53507 | Pseudomonas sp. JR1 |
| ipbaA | AF006691 | Pseudomonas putida RE204 |
| cumA1 | D37828 | Pseudomonas fluorescens IP01 |
| edoA1 | AF049851 | Pseudomonas fluorescens CA-4 |
| Naphthalene | | |
| dioxygenase | | |
| nahAc | M83949 | Pseudomonas putida G7 |
| doxB | M60405 | Pseudomonas sp. C18 |
| nahAc | U49496 | Pseudomonas sp. 9816-4 |
| ndoC2 | AF004284 | Pseudomonas putida ATCC 17484 |
| pahAc | AB004059 | Pseudomonas putida OUS82 |
| pahA3 | D84146 | Pseudomonas aeruginosa PaK1 |
| nahA3 | AF010471 | Pseudomonas putida BS202 |
| nagAc | AF036940 | Pseudomonas sp. U2 |
| dntAc | U62430 | Burkholderia sp. DNT |
| phnAc | AF061751 | Burkholderia sp. RP007 |
| phnAc | AB024945 | Alcaligenes faecalis AFK2 |
| nahAc | AF053737 | Cycloclasticus sp. 1P-32 |
| nahAc | AF053736 | Neptunomonas naphthovorans |
| | | NAG-2N-126 |
| nahAc | AF053735 | Neptunomonas naphthovorans |
| | | NAG-2N-113 |
| nahAc | AF093000 | Cycloclasticus sp. W |
| nahAc | AF092998 | Cycloclasticus pugetii PS-1 |
| narAc | AF082663 | Rhodococcus sp. NCIMB12038 |
| nahAc | AF039533 | Pseudomonas stutzeri AN10 |
| Toluene | | |
| dioxygenase | | |
| todC1 | J04996 | Pseudomonas putida F1 |
| bedC1 | L04642 | Pseudomonas putida ML2 |
| tcbAa | U15298 | Pseudomonas sp. P51 |

TABLE 2
Aromatic Monooxygenase Genes used to Deduce
Conserved Regions for PCR Primers.

| | Accession | |
|---|---|---|
| Gene | Number | Source Organism |
| Toluene monooxygenase | | |
| xylM | D63341 | Pseudomonas putida mt-2 |
| xylM | AF019635 | Pseudomonas putida HS1 |
| ntnM | AF043544 | Pseudomonas sp. TW3 |
| Ring hydroxylating | | |
| monooxygenases | | |
| tmoF | M95045 | Pseudomonas mendocina KR1 |
| tbuA1 | U04052 | Ralstonia picketti PKO1 |
| tbhA | AF001356 | Burkholderia cepacia AA1 |
| tbmD | L40033 | Pseudomonas sp. JS150 |
| bmoA | D83068 | Pseudomonas aeruginosa JI104 |
| Phenol hydroxylase | | |
| dmpN | M60276 | Pseudomonas putida CF600 |
| phhN | X79063 | Pseudomonas putida P35X |
| phenol hydroxylase | AB016863 | Comomonas testosteroni R2 |
| alpha subunit | | |
| phenol hydroxylase | AB016862 | Comomonas sp. E6 |
| alpha subunit | | |
| phenol hydroxylase | AB016861 | Burkholderia cepacia E1 |
| alpha subunit | | |
| phenol hydroxylase | AB016859 | Pseudomonas putida P-6 |
| alpha subunit | | |
| phenol hydroxylase | AB016858 | Pseudomonas putida P-8 |
| alpha subunit | | |
| poxD | AF026065 | Ralstonia sp. E2 |

PCR Primers and Conditions

PCR primers were chosen from conserved regions in the DNA sequences observed during alignments of each group of aromatic oxygenases. A description of the PCR primers and conditions is shown in Tables 3 and 4, respectively. The following combinations of primers were also used for multiplex PCR: PHE/NAH, TOL/TOD, and BPH2/BPH4. Annealing temperatures for multiplex PCR were 49, 55, and 62° C., respectively. All PCR mixtures contained 1×PCR buffer (Promega, Madison, Wis.), 0.2 mM of each dNTP (Amersham Pharmacia, Piscataway, N.J.), and 1 U Taq polymerase. Annealing temperature and, DNA (10, 1, 0.1 ng), MgCl₂, and primer concentrations were optimized for each primer set. MgCl₂ concentrations were increased from 1.5 to 3.0 mM until yield decreased or failed to increase. Primer concentrations were increased from 0.1 to 0.5 μM, except for BPH1 which was also tested at lower concentrations. Conventional and multiplex PCR was performed in a PTC-100 Programmable Thermal Controller (MJ Research, Inc., Waltham, Mass.) with the following temperature program: 10 min at 95° C. followed by 30 cycles of 1 min at 95° C., 1 min at optimum annealing temperature, 2 min at 72° C., after which a final extension step was conducted at 72° C. for 10 min. All experiments included control reactions without added DNA. PCR products were routinely visualized by running 10 μL of PCR mixture on 1% agarose gels (Bio-Rad, Richmond, Calif.) in 1× Tris-Acetate-EDTA (TAE) buffer stained with ethidium bromide (0.0001%). Reproducibility was confirmed by performing PCR with positive control DNAs in triplicate as a minimum.

TABLE 3

PCR Primers for Conventional, Multiplex, and Real-Time PCR.

| Primer | | | |
|---|---|---|---|
| Name | Target | SEQ ID NO | Sequence |
| NAH-F | Naphthalene | SEQ ID NO:1 | 5′-CAAAA(A/G)CACCTGATT(C/T)ATGG |
| NAH-R | Dioxygenase | SEQ ID NO:2 | 5′-A(C/T)(A/G)CG(A/G)G(C/G)GACTTCTTTCAA |
| TOD-F | Toluene | SEQ ID NO:3 | 5′-ACCGATGA(A/G)GA(C/T)CTGTACC |
| TOD-R | Dioxygenase | SEQ ID NO:4 | 5′-CTTCGGTC(A/C)AGTAGCTGGTG |
| TOL-F | Xylene | SEQ ID NO:5 | 5′-TGAGGCTGAAACTTTACGTAGA |
| TOL-R | Monooxygenase | SEQ ID NO:6 | 5′-CTCACCTGGAGTTGCGTAC |
| BPH1-F | Biphenyl | SEQ ID NO:7 | 5′-GGACGTGATGCTCGA(C/T)CGC |
| BPH1-R | Dioxygenase | SEQ ID NO:8 | 5′-TGTT(C/G)GG(C/T)ACGTT(A/C)AGGCCCAT |
| BPH2-F | Biphenyl | SEQ ID NO:9 | 5′-GACGCCCGCCCCTATATGGA |
| BPH2-R | Dioxygenase | SEQ ID NO:10 | 5′-AGCCGACGTTGCCAGGAAAAT |
| BPH3-F¹ | Biphenyl | SEQ ID NO:11 | 5′-CCGGGAGAACGGCAGGATC |
| BPH4-F | Dioxygenase | SEQ ID NO:12 | 5′-AAGGCCGGCGACTTCATGAC |
| BPH3-R | | SEQ ID NO:13 | 5′-TGCTCCGCTGCGAACTTCC |
| RMO-F | Toluene | SEQ ID NO:14 | 5′TCTC(A/C/G)AGCAT(C/T)CAGAC(A/C/G)GACG |
| RMO-R | Monooxygenase | SEQ ID NO:15 | 5′-TT(G/T)TCGATGAT(C/G/T)AC(A/G)TCCCA |
| RDEG-F | Toluene | SEQ ID NO:16 | 5′-T(C/T)TC(A/C/G)AGCAT(A/C/T)CA(A/G)AC(A/C/G)GA(C/T)GA |
| RDEG-R | Monooxygenase | SEQ ID NO:17 | 5′-TT(A/G/T)TCG(A/G)T(A/G)AT(C/G/T)AC(A/G)TCCCA |
| PHE-F | Phenol | SEQ ID NO:18 | 5′-GTGCTGAC(C/G)AA(C/T)CTG(C/T)TGTTC |
| PHE-R | Monooxygenase | SEQ ID NO:19 | 5′-CGCCAGAACCA(C/T)TT(A/G)TC |

¹BPH3-R is used with BPH3-F and BPH4-F.

TABLE 4
PCR Conditions for Conventional and Real-Time PCR.

| | | MgCl₂ | Primer | | Expected |
|---|---|---|---|---|---|
| | Tₐ¹ | Concentration | Concentration | Tₚ² | Product |
| Primer Name | ° C. | mM | μM | ° C. | Size (bp) |
| NAH-F | 47 | 2.5 | 0.3 | 83 | 377 |
| NAH-R | | | | | |
| TOD-F | 53 | 2.0 | 0.5 | 83 | 757 |
| TOD-R | | | | | |
| TOL-F | 55 | 2.5 | 0.2 | 82 | 475 |
| TOL-R | | | | | |
| BPH1-F | 57 | 2.0 | 0.06 | 88 | 671 |
| BPH1-R | | | | | |
| BPH2-F | 63 | 2.5 | 0.1 | 88 | 724 |
| BPH2-R | | | | | |
| BPH3-F | 62 | 1.5 | 0.1 | 87 | 570 |
| BPH4-F | 63 | 1.5 | 0.4 | 87 | 452 |
| BPH3-R | | | | | |
| RMO-F | 53 | 3 | 0.4 | 82 | 466 |
| RMO-R | | | | | |
| RDEG-F | 52 | 3 | 0.5 | 87 | 466 |
| RDEG-R | | | | | |
| PHE-F | 49 | 4 | 0.3 | 86 | 206 |
| PHE-R | | | | | |

¹Tₐ is the annealing temperature.
²Tₚ is the polymerization temperature used during real-time PCR.

Real-time PCR

Real-time PCR was performed on an ABI 7700 Sequence Detector (Applied Biosystems, Foster City, Calif.). Quantitative PCR reactions contained 1× Cloned Pfu Buffer (Stratagene, La Jolla, Calif.), 0.2 mM of each dNTP, SYBR Green (1:30000, Molecular Probes, Eugene, Oreg.), and 1U PfuTurbo HotStart DNA polymerase (Stratagene). Annealing temperatures, primer concentrations, and MgCl₂ concentrations for real-time PCR were the same as conventional PCR (Tables 3 and 4). To determine the melting temperature of amplification products, melting curves were acquired by heating to 95° C. for one minute, cooling to 5° C. below the annealing temperature, and heating at 0.2° C./s to 95° C. with fluorescence measurement taken during the final temperature ramp. The temperature of the extension step in subsequent PCR reactions was set at 4 to 5° C. below the observed melting temperature.

Threshold Cycle Number Calculation

The Sequence Detector program (Applied Biosystems, version1.7) subtracted background signal for each sample determined during cycles 3 through 15. The fluorescence threshold was computed as ten times the standard deviation of the background signals and fractional cycle numbers were computed that correlate inversely to the log of the initial template concentration. The best fit (by method of least squares) was then used to plot the standard curve. The lower detection was defined as the lowest template concentration which resulted in a threshold cycle that was significantly less than the total number of cycles (α=0.05).

Gene Probe Construction

Probes were generated by PCR incorporation of DIG-labeled dUTP (digoxigenin 11-dUTP) into the amplicon for each primer set with positive control template. All of the PCR reagents described previously were added for the creation of the probes except digoxigenin 11-dUTP was partially substituted for dTTP in the nucleotide mixture. A DIG-dUTP/dTTP ratio of 1:3 was used with the total concentration (of dTTP and dUTP) remaining 0.2 mM.

Alkaline Transfer of PCR Products to Nylon Membranes

Hybridization studies with PCR products of each primer set with positive and negative control DNA were performed to confirm specificity. First, PCR products were separated on a 1% agarose gel and photographed beside a ruler so that fragments detected following hybridization could be matched to fragments on the gel. Once photographed, the gel was shaken in 0.25 N HCl solution to depurinate the DNA allowing easier transfer to the membrane.

During the depurination step, the alkaline transfer apparatus was constructed. A plastic tray was filled with alkaline transfer solution (0.4 N NaOH). A glass plate was then placed over most of the tray. Then a wick (7 cm×22 cm) made of 3MM filter paper was cut, wetted in transfer solution, and placed on the glass plate so that the ends were submerged in the transfer solution. Any bubbles were rolled out with a pipette. The gel was then removed from the depurination solution, rinsed with water, and gently placed upside down on the wick. Again bubbles were removed. Strips of parafilm approximately an inch in width were cut and placed along each side of the gel. The nylon membrane (Hybond-N+, Amersham Pharmacia) was then carefully placed atop the gel using blunt end forceps. Once the membrane was in place, two pieces of 3MM filter paper cut to the size of the gel were soaked in the transfer solution and placed on to of the membrane. Bubbles were removed by rolling with a pipette. Next, two additional dry pieces of 3MM filter paper were placed on top the previous two and bubbles were removed. Finally, a stack of paper towels (between 5 cm and 6 cm) was added to drive the capillary action. A glass plate and an Erylenmeyer flask containing approximately 50 ml of water were used to weigh down the paper towels. DNA was allowed to transfer overnight (approximately 16 hours).

Following transfer, the alkaline blot apparatus was disassembled in the opposite order. Before the membrane was removed, however, the positions of the well were marked with a soft lead pencil. The membrane was then placed in a buffer solution to be neutralized (0.5 M Tris-Cl/1 M NaCl) and remove any remaining pieces of agarose gel. The membrane was then ready for prehybridization.

Prehybridization and Hybridization of Membranes

Membranes were then prehybridized to block sites on the membrane that did not contain DNA to prevent the labeled probe from binding thus creating background signal. The prehybridization solution contained 5×SSC, 0.1% N-lauroylsarcosine, 0.02% sodium dodecylsulfate, 50% formamide, and 2× blocking reagent (Boehringer-Mannheim, Indianapolis, Ind.). For a 100 cm² membrane, 20 ml of prehybridization solution was used. The membrane was soaked in the prehybridization solution in the hybridization oven at 25° C. below the predicted melting temperature of the probe for 8-10 hours. Near the end of the prehybridization period, the hybridization solution was prepared. The hybridization solution was heated to 65° C. for 10 minutes to denature the probe and then quickly chilled on ice for two minutes. The prehybridization solution was poured off the membrane and frozen for future use. The hybridization solution was then added to the membrane and the blot was incubated in the hybridization oven overnight. Hybridization was performed at 25° C. below the predicted melting temperature.

Detection of Membranes

Probes were detected according to manufacturer's instructions (Roche Molecular Biochemicals) briefly outlined below. Following hybridization, the probe solution was poured off the membrane and stored at −20° C. for future use. The membrane was then washed for 5 minutes at room temperature in the low stringency wash solution (2×SSC, 0.1% SDS). Hybridizations were performed under high- and low-stringency conditions by adjusting the temperature of the following post-hybridization washes (Nakatsu, C. H. and Forney. 1996. Parameters of nucleic acid hybridization experiments. Molecular Microbial Ecology Manual 2.1.2: 1-12.). The membrane was washed twice for 15 minutes in 0.1×SSC, 0.1% SDS at 10° C. below the predicted melting temperature (high stringency) or 30° C. below the predicted melting temperature (low stringency). This wash step was responsible for removing any probe that was not bound to the DNA transferred to the membrane. The remaining steps are described for a 100 cm² blot and were performed at room temperature. The membrane was next washed for 1 minute in buffer 1 (100 mM maleic acid, 150 mM NaCl, pH 7.5). Then the blot was incubated for 30 minutes in 30 ml of blocking solution (1× blocking solution in buffer 1). As the name suggests, the blocking reagent prevented nonspecific binding of the antibody conjugate to the membrane. Next the membrane was incubated for 30 minutes in the antibody solution containing Anti-Digoxigenin conjugated to alkaline phosphatase. The actual detection is based on this antibody binding to the digoxigenin-labeled probe. The membrane was then washed twice in buffer 1 for 15 minutes (per wash) to remove any unbound antibody conjugate. Finally, the membrane was washed in buffer 3 (100 mM Tris-Cl, pH 9.5, 100 mM NaCl, 50 mM MgCl₂) for 2 minutes and carefully placed in a Seal-A-Meal bag. To the blot, 5 ml of color solution containing NBT (nitroblue tetrazolium salt) and BCIP (5-bromo-4-chloro-3-indoyl phosphate in buffer 3 was added. A pipette was used to gently roll out any bubbles in the solution before the bag was sealed. Detection was based on the development of a blue/purple color due to an interaction between NBT, BCIP, and bound antibody conjugate. Color development was allowed for 2 hours to overnight (16 hours) with periodic checking. To stop the color reaction the membrane was washed in TE. The blot was then photographed with a ruler at the side to distinguish products.

Results

Phylogeny of the Large Subunits of Aromatic Oxygenases

The large subunits of aromatic dioxygenases (FIG. 1) and monooxygenases (FIG. 2) with the same reported substrate specificity are, in general, closely related but distinct types are evident. The first type (N), consisting primarily of naphthalene dioxygenases, contains two families (N.1 and N.2) each with multiple subfamilies. Naphthalene dioxygenase specific primers (NAH) were identified to target the N.2.A subfamily of naphthalene dioxygenases with high sequence identity to nahAc from P. putida G7. The dntAc from Burkholderia sp. DNT belongs to this phylogenetic subfamily as indicated by the relatively high DNA sequence identity to the naphthalene dioxygenase gene nagAc of Pseudomonas sp. U2 (92.1%). Furthermore, clones expressing dinitrotoluene dioxygenase have been reported to convert naphthalene to the corresponding cis-dihydrodiol. The other naphthalene dioxygenase subfamilies not targeted by the NAH primers are sequences from marine isolates and the PAH-attacking dioxygenases from Alcaligenes faecalis AFK2 and Burkholderia sp. RP007. An additional non-target sequence, narAa from Rhodococcus sp. strain NCIMB 12038, appears to be more closely related to biphenyl and toluene dioxygenases than other naphthalene dioxygenases.

The second type of aromatic dioxygenase is composed of 2 families of biphenyl and mono-aromatic dioxygenases (FIG. 1, D.1 and D.2). The sequence similarity and functional overlap of biphenyl and alkyl-benzene dioxygenases including toluene dioxygenase has been noted previously. The D.1 family includes two subfamilies of biphenyl dioxygenases from gram negative organisms (D.1.B and D.1.C) and a subfamily of monoaromatic dioxygenase genes (D.1.A). The second family (D.2) is comprised of 2 subfamilies of biphenyl dioxygenases from gram positive organisms (D.2.A and D.2.B) and a subfamily of toluene dioxygenases (D.2.C). The D.2.B subfamily included ipbA1 from R. erythropolis BD2 which had a higher percent DNA sequence identity to bphA1 from Rhodococcus sp. RHA1 than to isopropylbenzene dioxygenases from gram negative organisms. Separate BPH primer sets were identified to detect and distinguish between all four biphenyl dioxygenase subfamilies as shown in FIG. 1. The BPH4 primers were identified to allow amplification of biphenyl and isopropylbenzene dioxygenases within the D.2 family, whereas the BPH3 primers are specific for the D.2.A subfamily. The D.2.C subfamily of dioxygenases for toluene, benzene, and chlorobenzene degradation, are closely related and were used to deduce toluene dioxygenase specific primers (TOD).

Alignments were also constructed for the large subunits of toluene monooxygenase genes. The two types revealed with this alignment (FIG. 2) differed in their mode of attack—ring-hydroxylating monooxygenases (R) and alkyl-group hydroxylating monooxygenases (T). With two exceptions, the ring-hydroxylating monooxygenases were divided into families based on substrate specificity: two families of aromatic hydrocarbon-attacking monooxygenases (R.2 and R.3) and one family of phenol hydroxylases (R.1). The two exceptions are phlK from Ralstonia eutropha JMP134 which grouped with the toluene monooxygenases and tbmD from Burkholderia sp. strain JS150 which is more closely related to the phenol hydroxylases. It has been previously shown that the tbm operon from JS150 has the same gene arrangement and strong sequence identity to the phenol hydroxylases encoded by dmp, phe, and phh of CF600, BH, and P35X, respectively. Moreover, tbmD has also been shown to be responsible for oxidation of o-cresol produced from the initial hydroxylation of toluene supporting the association of this family with oxidation of hydroxylated substrates. Members of the R.2 and R.3 families will oxidize hydroxylated intermediates in addition to toluene (touA). PhlK has been described as a phenol hydroxylase but its specificity has not been rigorously determined. Thus it may also be active in toluene oxidation and belong to this phylogenetic family. Based upon the apparent phylogeny, four primer sets were identified to detect each family as shown (FIG. 2). RDEG primers were designed to amplify families R.2 and R.3 whereas the RMO primers are specific for the R.2 family.

Because isolation of aromatic hydrocarbon-degrading organisms has traditionally relied on selective enrichment that has been shown to reduce diversity, the phylograms described in FIGS. 1 and 2 represent the diversity only of currently known aromatic oxygenase gene sequences, and not the true environmental diversity. As the number of available sequences increases, further conserved regions used for primers can be identified or refinements to primer selection can be made to accommodate additional information. By refining and/or developing new primers, sequence variability in a polluted soil sample can be more thoroughly assessed.

PCR Primer Testing and Optimization

To test specificity, PCR amplification with each primer set was performed with DNAs from positive control strains containing the target and negative controls containing other oxygenase genes (Table 5). For each primer set, amplification with positive control DNA yielded amplification products of the predicted size. In most cases, no products were observed with negative control template DNAs, however, a few exceptions were noted. With reactions containing NAH primers and P. putida HS1 DNA as the template, an approximately 850 bp product was observed which did not hybridize to the NAH/G7 probe.

TABLE 5
Summary of results for PCR amplification and hybridization with positive and negative control DNAs.

| | | P. | | C. | P. | P. | | P. | P. | |
|---|---|---|---|---|---|---|---|---|---|---|
| Primer | P. putida | putida | P. putida | testosteroni | pseudoalcaligenes | erythropolis | Rhodococcus | aeruginosa | mendocinsa | Pseudomonas |
| Set | G7 | F1 | HS1 | B-356 | KF707 | TA421 | sp. RHA1 | JI104 | KR1 | sp. CF600 |
| NAH | ++ | — | * | — | — | — | — | | — | — |
| TOD | — | ++ | — | — | — | — | — | | — | — |
| TOL | — | — | ++ | — | — | — | — | | — | — |
| BPH1 | — | — | — | ++ | — | — | — | | — | — |
| BPH2 | — | — | — | — | ++ | — | — | ++ | — | — |
| BPH3 | — | — | — | — | — | ++ | — | | — | — |
| BPH4 | — | — | — | — | — | ++ | ++ | | — | — |
| RMO | — | — | — | — | — | — | — | ++ | + | — |
| RDEG | — | — | — | — | — | — | — | + | ++ | — |
| PHE | — | — | — | — | — | — | — | ++ | ++ | ++ |

(++) Amplification product was observed when 10 μL of PCR product were run on a 1% agarose gel and the product hybridized to corresponding probe,
(+) product of the proper size was observed which did not hybridize to the corresponding probe until the stringency was reduced to approximately 60%,
(—) no amplification or hybridization products were observed,
(*) product observed of an unexpected size which did not hybridize to corresponding probe.

PCR with the RMO primers produced an amplicon of approximately 466 bp with P. mendocina KR1 despite two predicted mismatches with each primer. The product weakly hybridized with the RMO probe constructed from JI104 template when the stringency was reduced to approximately 60%. Amplicons characteristic of a phenol hydroxylase gene were observed in reactions with PHE primers and KR1 and JI104 DNAs. The product resulting from KR1 template hybridized under medium stringency conditions to the PHE/CF600 probe whereas the JI104 product did not hybridize to the probe until the stringency was reduced to approximately 60%. A product characteristic of the BPH2 subfamily of biphenyl dioxygenases was also observed with JI104. Since the product hybridized to the BPH2/KF707 probe under high stringency conditions and biphenyl will support its growth, JI104 is believed to contain a bph operon in addition to the known bmo operon.

Multiplex PCR with Positive Control DNAs

Combinations of primer sets based on optimum annealing temperatures were tested in multiplex PCR to allow simultaneous detection and consequently faster sample processing. Although the range of primer annealing temperatures excluded many combinations, the PHE/NAH, TOL/TOD, and BPH2/BPH4 primer sets allowed reliable detection in multiplex PCR experiments.

Product yields with the TOL and TOD primers and the BPH2 and BPH4 primers were unchanged in multiplex reactions. Amplification of the PHE product was reduced slightly in multiplex reactions judging from product intensity, however, products could still be observed with template concentrations of 0.1 ng per reaction (10⁵ copies per reaction) in a 10 fold excess of P. putida G7 template.

Real-Time PCR Amplification

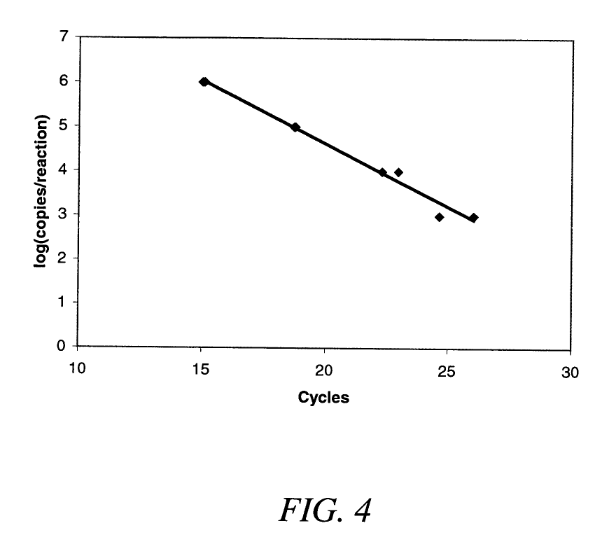

To determine how easily real-time PCR could be conducted with existing primers, real-time PCR experiments were initially performed with the optimum conditions determined by conventional PCR. For some primer sets no modifications of PCR conditions were needed and a log-linear relationship was observed between copy number and threshold cycle (Cₜ). For others a significant fluorescence signal was observed for no template control samples. After determining that the signal in no DNA controls was not a result of contamination, PfuTurbo Hotstart DNA polymerase was used to decrease formation of any nonspecific products. This measure alone did not eliminate background signal, therefore, melting curves were developed to aid in choosing a polymerization temperature. The large change in fluorescence signal during the temperature ramp occurs at the melting point of the desired product (92° C.). By collecting fluorescence data at extension temperatures near the melting temperature of the desired product, fluorescence signals from primer dimers and non-specific sources were greatly reduced. Temperatures of the extension step (Table 4) were as high as 88° C. which is considerably higher than 72° C. commonly used. Following optimization, standard curves were developed with known template concentrations. For all primer sets, a log-linear relationship was found between copy number and Ct for template concentrations ranging from 10⁷ to 10³ copies per reaction (FIG. 4). Template concentrations greater than 10⁷ copies inhibited amplification as judged by increasing Cₜ values for 50 ng samples. The lower detection limit was copies per reaction for all primer sets.

Based on the sequence alignments, the 2-nitrotoluene dioxygenase primers would be expected to amplify some but perhaps not all naphthalene dioxygenase genes from the N.2.A subfamily. As mentioned previously, the NAH primers did generate a product of approximately 850 bp with P. putida HS1 DNA. Amplification of a fragment of the toluene monooxygenase gene xylM with the NAH primers seems unlikely since no products were observed when DNA from P. putida mt-2 was used. Although unexpected, this product was easily distinguished from the NAH product and did not generate false positive results with environmental samples (Example 2). Admittedly, the NAH primers described here can be used to detect only a subset of naphthalene dioxygenase genes, however, detection of this subfamily may be an indicator of naphthalene catabolic ability. Furthermore, additional primer sets can be provided as described herein to detect functionally similar genotypes not detected by the tested NAH primers. For example, to expand the range of naphthalene catabolic genes detected, phnAc primers based upon the sequence from Burkholderia sp. RP007 could be used in conjunction with nahAc primers.

Unlike naphthalene dioxygenase, relatively little has been published concerning PCR primers targeting other aromatic oxygenase genes and many of these target species-specific genes not an entire subfamily. Species specific primer sets have been described which amplify fragments of todC1 from P. putida F1 and toluene-4-monooxygenase (tmoAa) from P. mendocina KR1. These primers may not amplify benzene, toluene, and chlorobenzene dioxygenase genes related to todC1 or the R.2 family of toluene monooxygenases related to tmoA however (FIG. 2). Biphenyl dioxygenase-specific primers have also been reported, however, sequence alignments with todC1 only indicated 2 mismatches suggesting that toluene dioxygenase genes may also be amplified by this primer set.

The PHE primers adequately detect phenol hydroxylase genes from family R.1; however, unexpected amplification products were noted with P. aeruginosa JI104 and P. mendocina KR1 from R.2 and R.3, respectively. There should be sufficient mismatches between the PHE primers and the monooxygenase genes to prevent amplification, therefore, it is believed that a gene downstream in the pathway was possibly amplified. Because both strains produce methyl-substituted phenols from toluene, downstream phenol hydroxylases would seem likely.

### Example Two

The objective of this study was two-fold: (1) to determine the effect of aromatic hydrocarbon contamination on a soil system in terms of community structure and function and (2) to test the primer sets described in Example 1 with environmental isolates and total soil DNA. The detection of oxygenase genes in soil DNA extracts from the microcosms demonstrates the utility of the primers developed in Example 1 with uncharacterized microbial populations and provides insight into the selection of genotypes in the environment.

Materials and Methods

Soil microcosms spiked with individual aromatic hydrocarbons were prepared to investigate the selective pressure exerted by aromatic hydrocarbon-contamination on the indigenous microbial community in soil. Presumably, the addition of an aromatic compound would select for bacteria capable of utilizing the compound as a carbon and energy source. For some aromatic hydrocarbons, biochemical pathways (and in some cases multiple pathways) have been elucidated which led to their biodegradation, however, their prevalence in the environment has not been thoroughly assessed. Individual aromatic hydrocarbons were used to determine the effect of specific pollutants on the community structure and function. Each week, microcosm samples were taken for isolate pure cultures and soil DNA extraction. Pure cultures were isolated from the plating experiments for genotype screening. Screening of the environmental isolates was performed to examine the selective pressure exerted by each substrate on the culturable portion of the community and to test the PCR primers with uncharacterized isolates. Genotype screening of soil DNA extracts allowed investigation of the total bacterial community in terms of catabolic genes. PCR-DGGE profiles were used to examine community structure and coupled to genotype screening of the environmental isolates allowed us to investigate dominant members of the community. Details of the procedures used are presented in the following sections.

Soil Microcosms

A sandy loam soil was collected from a site in Valporaiso, Ind. with no known prior exposure to aromatic hydrocarbons. For each microcosm, five grams of sieved soil (2 mm sieve) was combined with 20 ml of sterile minimal medium in a sterile 120 ml crimp top serum vial. The microcosms were then spiked with one of several aromatic hydrocarbons as the enrichment substrate (Table 6).

TABLE 6
Aromatic Enrichment Substrates in Soil Microcosms.

| | Calculated | Calculated | |
|---|---|---|---|
| Enrichment | Concentration | Carbon Flux | Solubility |
| Substrate | (μg l⁻¹) | (μg C g⁻¹ soil wk⁻¹) | (μg l⁻¹) |
| benzene¹ | 330 | 7.3 | 1,790,000 |
| toluene (low)¹ | 250 | 5.5 | 518,000 |
| toluene (high)¹ | 7,500 | 164.0 | 518,000 |
| o-xylene¹ | 225 | 4.9 | 185,000 |
| m-xylene¹ | 7,100 | 154.3 | 160,000 |
| p-xylene¹ | 210 | 4.6 | 180,000 |
| naphthalene² | 31,500 | | 31,500 |
| biphenyl² | 7,000 | | 7,000 |
| phenanthrene² | 1,100 | | 1,100 |
| gasoline | saturated | | not |
| | vapor phase | | applicable |

¹Concentrations were calculated based on a three phase system using literature values for aqueous solubility (Mackay et al., 1992; Schwarzenbach et al., 1993).
²Pure compound aqueous solubility limit.

Due to potential toxicity at high concentrations, the mono-aromatic compounds were added well below their pure compound solubilities. Furthermore, these concentrations are more likely to reflect field conditions in which these compounds are present as components of petroleum products. Except for the gasoline microcosms, each vial was crimp sealed after addition of the hydrocarbon substrate. Gasoline was provided to some soil microcosms as described by Ridgway et al. (Ridgway, H. F., J. Safarik, D. Phipps, P. Carl, and D. Clark. 1990. Identification and catabolic activity of well-derived gasoline degrading bacteria from a contaminated aquifer. Applied and Environmental Microbiology 56:3565-3575.). Briefly, gasoline soil microcosms were kept in sealed containers with an autosampler vial containing 1 ml of gasoline whose septum had been repeatedly pierced. The Teflon-coated septa of these microcosms were pierced with a syringe needle allowing gasoline vapors and oxygen to enter the microcosm. To test if oxygen was needed to enrich for bacteria which use aromatic oxygenase genes, anaerobic microcosms were prepared. These microcosms were purged with nitrogen and supplied with naphthalene as the carbon source. An additional pair of unamended microcosms were prepared to serve as a basis for comparison. All microcosms were prepared in duplicate and sampled weekly. All microcosms were incubated in the dark at room temperature for four weeks. All microcosms were gently shaken daily to mix and aerate the soil slurry.

Microcosm Sampling

Weekly samples were taken from soil microcosms for cultivation experiments and DNA extraction. A vial was gently shaken and then 0.5 ml of the soil slurry was removed with a glass Pasteur pipette. This aliquot was used for DNA extraction using the BIO101 soil DNA extraction protocol (Soil DNA Extractions). One milliliter aliquots were removed in an analogous manner for cultivation experiments. Following sampling, each microcosm was respiked with substrate, capped, and crimp-sealed.

Soil DNA Extractions

Total soil DNA was extracted using the FastDNA SpinKit for Soil (QbioGene, Carlsbad, Calif.). The 0.5 ml aliquots of soil slurry were first added to MULTIMIX 2 Tissue Matrix Tubes. These tubes contain small beads to aid in mechanical cell disruption. One milliliter of lysis buffer was then added to each tube and cell lysis was achieved by homogenizing the soil slurry in the FastPrep Instrument for 30 seconds at a speed of 5.5. Lysis buffer contained (in 200 ml) 1.36 ml of 1 M NaH₂PO₄, 18.64 ml of 1 M Na₂HPO₄, and 10 g of sodium dodecylsulfate (SDS). The combination of the chemical action of the lysis buffer and the mechanical abrasion during homogenization disrupted cell walls and membranes releasing DNA into solution. The intense shaking of the FastPrep Instrument caused, in some cases, excessive heating of the plastic tubes which in turn caused failure of the tubes during centrifugation. To avoid this problem, tubes were incubated on ice for 5 minutes following homogenization. Then the tubes were centrifuged for 30 seconds at 14,000×g and the supernatant was transferred to fresh tubes. Protein precipitation solution (250 μl) was added and the tubes were mixed by hand. Next the tubes were centrifuged at 14,000×g for 5 minutes to pellet the precipitated proteins and the supernatant was transferred to fresh tubes. Then 1 ml of Binding Matrix was added, the solution was inverted slowly by hand for two minutes to provide gentle mixing. Using a pipette, 500 μl aliquots of the Binding Matrix solution were transferred to Spin Filters which retained the Binding Matrix but allowed liquid to pass into the Catch Tubes during centrifugation. The Binding Matrix was washed with 500 μl of New Wash solution and the tubes were centrifuged for one minute at 14,000×g. New Wash is 14 ml NewWash Concentrate, 280 ml water, and 310 ml ethanol. The tube was centrifuged for an additional two minutes at 14,000×g to remove the last of the New Wash solution. The pellet was air dried for approximately 15 minutes. The Spin Filter containing the DNA Binding Matrix was then transferred to a fresh Catch Tube, 50 μl of TE was added to resuspend the pellet, and centrifuged for one minute. The DNA bound to the Binding Matrix was eluted by the TE. This was repeated with a second 50 μl of TE.

Cultivation of Bacteria from Soil Microcosms

Weekly 1 ml samples were taken from each microcosm for to obtain pure cultures. The 1 ml aliquots were serially diluted in minimal media and 0.1 ml samples from each dilution were plated on minimal media plates amended with the enrichment substrate as the sole source of carbon and energy. Plates were incubated at room temperature for 1 week. Individual colonies were picked from these plates by sterile toothpick and transferred to fresh plates. Cultures were named according to enrichment (E) substrate or direct (D) isolation substrate, dilution number, week number, and isolate number. Cultures were streaked to purity as needed. Each culture was then grown in minimal liquid media with the isolation substrate as the carbon source. Following incubation, 1.7 ml aliquots of pure cultures were placed in cryovials containing 0.3 ml of glycerol and frozen at −80° C. for long-term storage.

Screening Environmental Isolates by REP-PCR

REP-PCR was performed on environmental isolates to obtain genomic fingerprints that could be used to distinguish siblings prior to catabolic oxygenase screening. Although their function remains unclear, consensus REP sequences (repetitive extragenic palindromic) have been detected in a large variety of bacterial genera. With REP sequences as primers and total genomic DNA as the template, REP-PCR generates products which when run on an agarose gel yield a characteristic pattern for each unique strain. REP-PCR was performed using primers and reaction conditions as described by deBruijn (deBruijn, F. J. 1992. Use of repetitive (repetitive extragenic palidromic and enterobacterial repetitive intergeneric consensus) sequences and the polymerase chain reaction to fingerprint the genomes of Rhizobium meliloti isolates and other soil bacteria. Applied and Environmental Microbiology 58:2180-2187.). Each culture was revived from glycerol storage by plating on minimal media plates containing the isolation substrate. Single colonies from these plates were used to inoculate 5 ml liquid cultures. Usually following overnight or two days of incubation, the liquid cultures were frozen at −20° C. One milliliter of these cells was used as the template for REP-PCR. A genetic distance was calculated for pairs of likely siblings and between isolates considered to be unique. The genetic distance was calculated by determining the number of different PCR fragments (either present or absent) and dividing by the total number of PCR fragments for the two isolates. Isolates were considered siblings if the genetic distance was less than 0.33 because clones from the same evolved population can have genetic distances of as high as 0.33 (Nakatsu, C. H., R. Korona, R. E. Lenski, F. J. deBruijn, T. L. Marsh, and L. J. Forney. 1998. Parallel and Divergent Genotypic Evolution in Experimental Populations of Ralstonia sp. Journal of Bacteriology 180:4325-4331.). In most cases, however, genetic distances for isolates considered siblings were less than 0.1.

Catabolic Genotype Screening of Environmental Isolates and Soil Microcosm DNA

Unique environmental isolates and soil microcosm DNA were screened for aromatic oxygenase genes by multiplex PCR amplification with aromatic oxygenase specific primers as described in Example 1.

Community Analysis by Denaturant Gradient Gel Electrophoresis (DGGE)

The variable V3 region of the 16S rRNA gene was amplified using PRBA338 primer (5′-ACTCCTACGGGAGGCAGCAG-3′) (SEQ ID NO: 20) and PRUN518R primer (5′-ATTACCGCGGCTGCTGG-3′) (SEQ ID NO: 21) with a GC clamp (Muyzer, G., E. C. De Waal, and A. G. Uitterlinden. 1993. Profiling of complex microbial populations by denaturing gradient gel electrophoresis analysis of polymerase chain reaction-amplified genes coding for 16S rRNA. Applied and Environmental Microbiology 59:695-700.). The PCR protocol was comprised of a 5-min initial denaturation at 94° C., 30 cycles of 92° C. for 30 s, 55° C. for 30 s, and 72° C. for 30 s followed by 15 min at 72° C. All reactions included 1×PCR buffer (Promega, Madison, Wis.), 175 μmol of MgCl₂, 4 nmol of deoxynucleoside triphosphates, 1% bovine serum albumin, 25 pmol (each) of forward and reverse primers, and 2 units of Taq polymerase. The different PCR products were resolved on 8% (wt/vol) polyacrylamide gels in 0.5×TAE (20 mM Tris-Cl, 10 mM acetate, 0.5 mM Na₂EDTA) using a denaturing gradient ranging from 32.5 to 57.5%. (where 100% denaturant contains 7 M urea and 40% formamide). Electrophoresis was performed at 60° C. and at 20 V (15 min) followed by 200 V for 5.5 hours. Gels were stained with SYBR Green I (1:5,000 dilution in TAE, Molecular Probes, Eugene, Oreg.) and visualized on a UV transilluminator.

Results

Approximately 30 pure cultures were isolated from each microcosm for a total of 205 pure cultures. On average, six unique strains based on REP-PCR pattern were isolated from each microcosm. Strains were then screened for the presence of aromatic oxygenase genes. Results are set forth in Table 7.

TABLE 7
Summary of Environmental Isolate Genotype Screening

| | Primer Set | | | | | | |
|---|---|---|---|---|---|---|---|
| Environmental | REP-PCR | | | | | | Isolation |
| Isolate | Group | PHE | NAH | TOL | RMO/RDEG | BPH4 | Substrate |
| DNT 3-0-5 | T1 | + | — | — | — | | toluene |
| ETT 3-2-27 | T5 | + | — | — | — | — | toluene |
| ETZ 3-2-2 | B2 | + | — | — | — | — | benzene |
| ETZ 4-2-4 | B3 | + | — | — | — | — | benzene |
| EGZ 3-3-9 | B4 | + | — | — | — | — | benzene |
| o-x-1 | O1 | + | — | — | + | — | o-xylene |
| o-x-4 | O1 | + | — | — | + | — | o-xylene |
| o-x-7 | O1 | + | — | — | + | — | o-xylene |
| o-x-9 | O1 | + | — | — | + | — | o-xylene |
| EXX 4-1-14 | M3 | + | — | + | — | — | m-xylene |
| p-x-11 | P1 | + | — | + | — | — | p-xylene |
| DNG 5-0-3 | G1 | + | — | — | — | — | gasoline |
| DNG 3-0-4 | G2 | + | — | — | — | — | gasoline |
| EGG 3-1-13 | G6 | + | — | + | — | — | gasoline |
| EGG 4-1-15 | G7 | + | — | — | — | — | gasoline |
| EGG 3-2-21 | G10 | + | — | — | — | — | gasoline |
| DNG 5-0-9 | G4 | + | — | — | — | — | gasoline |
| BPH1 | B1 | + | — | — | — | + | biphenyl |
| BPH3 | B3 | + | — | — | — | + | biphenyl |
| DNN 3-0-2 | N1 | — | + | — | — | — | naphthalene |
| ENN11 | N2 | — | + | — | — | — | naphthalene |
| ENN12 | N3 | — | + | — | — | — | naphthalene |
| ENN15 | N2 | — | + | — | — | — | naphthalene |
| ENN16 | N4 | — | + | — | — | — | naphthalene |
| ENN18 | N5 | — | + | — | — | — | naphthalene |
| ENN19 | N6 | — | + | — | — | — | naphthalene |
| ENN21 | N7 | — | + | — | — | — | naphthalene |
| ENN23 | N7 | — | + | — | — | — | naphthalene |
| ENN26 | N8 | — | + | — | — | — | naphthalene |
| ENN29 | N8 | — | + | — | — | — | naphthalene |
| ENN32 | N8 | — | + | — | — | — | naphthalene |
| ENN36 | N8 | — | + | — | — | — | naphthalene |
| DNX 3-0-1 | M1 | — | — | + | — | — | m-xylene |
| DNX 4-0-2 | M2 | — | — | + | — | — | m-xylene |
| DNX 4-0-8 | M3 | — | — | + | — | — | m-xylene |
| EXX 3-1-10 | M3 | — | — | + | — | — | m-xylene |
| EXX 3-1-11 | M3 | — | — | + | + | — | m-xylene |
| EXX 4-1-12 | M3 | — | — | + | — | — | m-xylene |
| EXX 4-4-25 | M5 | — | — | + | + | — | m-xylene |
| EXX 4-4-26 | M6 | — | — | + | — | — | m-xylene |
| EXX 5-4-31 | M5 | — | — | + | — | — | m-xylene |
| EXX 5-4-32 | M7 | — | — | + | — | — | m-xylene |
| EXX 4-4-35 | M8 | — | — | + | + | — | m-xylene |
| EXX 4-4-36 | M5 | — | — | + | + | — | m-xylene |
| EXX 4-4-38 | M9 | — | — | + | — | — | m-xylene |
| p-x-1 | P1 | — | — | + | — | — | p-xylene |
| p-x-4 | P1 | — | — | + | — | — | p-xylene |
| p-x-8 | P1 | — | — | + | — | — | p-xylene |
| EGG 3-1-12 | G5 | — | — | + | — | — | gasoline |
| EGG 3-2-20 | G9 | — | — | + | — | — | gasoline |
| EGZ 3-3-24 | B6 | — | — | — | — | + | benzene |
| BPH2 | BP2 | — | — | — | — | + | biphenyl |
| BPH5 | BP4 | — | — | — | — | + | biphenyl |
| BPH7 | BP5 | — | — | — | — | + | biphenyl |
| BPH8 | BP6 | — | — | — | — | + | biphenyl |
| BPH11 | BP7 | — | — | — | — | + | biphenyl |
| BPH12 | BP8 | — | — | — | — | + | biphenyl |
| DNT 4-0-3 | T2 | — | — | — | + | — | toluene |
| EGZ 3-3-10 | B5 | — | — | — | + | — | benzene |
| DNT 3-0-1 | T1 | — | — | — | — | — | toluene |
| ETT 3-1-11 | T4 | — | — | — | — | — | toluene |
| ETT 3-2-18 | T5 | — | — | — | — | — | toluene |
| DNG 4-0-5 | G3 | — | — | — | — | — | gasoline |
| EGG 4-1-17 | G8 | — | — | — | — | — | gasoline |
| EGG 4-2-23 | G11 | — | — | — | — | — | gasoline |

(+) denotes that the described product was observed and hybridized to appropriate probe.
(—) oxygenase product was not observed.

Oxygenase Screening of Environmental Isolates and Microcosms

Benzene and Toluene Experiments

Based on the oxygenase screening of benzene and toluene isolates as well as soil DNA from these microcosms, both substrates enriched PHE -harboring strains (Table 8). Products characteristic of ring-hydroxylating-monooxygenase genes were observed in each microcosm, but none of the products hybridized with the RMO/JI104 or RDEG/KR1 probes even when the stringency was reduced to approximately 60%. RMO products from one benzene isolate and one toluene isolate did hybridize to the RDEG/KR1 probe at low stringency conditions. Non-specific products were observed with RMO and RDEG primers and DNA from several isolates, but these products did not hybridize to the corresponding probes. For one of the benzene-utilizing isolates, the BPH4 subfamily of biphenyl dioxygenases was the only type of oxygenase gene detected with the array of primers used, but biphenyl dioxygenase genes were not detected in the benzene microcosm. Although not detected in benzene isolates, faint products were observed with benzene microcosm DNA and NAH primers which weakly hybridized to the NAH/G7 probe under low stringency conditions. TOL and TOD were not detected in any microcosm samples or isolates grown on benzene or toluene. Three of the toluene isolates did not contain oxygenase genes which could be detected with the methods used. A complete description of the genotype screening for each environmental isolate is shown in Table 7.

TABLE 8
Aromatic Oxygenase Genes Detected in Benzene and
Toluene Experiments.

| | | Primer Set | | | | | |
|---|---|---|---|---|---|---|---|
| | Total | | RMO/ | | | | |
| | Isolates | PHE | RDEG | TOL | NAH | BPH4 | TOD |
| benzene | 5 | 3 | 1 | 0 | 0 | 1 | 0 |
| isolates | | | | | | | |
| benzene | | + | * | — | + | — | — |
| microcosm | | | | | | | |
| toluene | 6 | 2 | 1 | 0 | 0 | 0 | 0 |
| isolates | | | | | | | |
| toluene | | ++ | * | — | — | — | — |
| microcosm | | | | | | | |
| (low) | | | | | | | |
| toluene | | ++ | — | — | — | — | — |
| microcosm | | | | | | | |
| (high) | | | | | | | |

(++) Product was observed during all four weeks and hybridized to positive control probe.
(+) Product was observed during three weeks and hybridized to positive control probe.
(*) Product of the correct size was observed in at least three weeks but did not hybridize to positive control probe.
(—) No product observed.

o-Xylene Experiments

RMO was consistently detected in the o-xylene microcosm and isolates (Table 9). For the isolates, the amplicons produced from PCR with RMO primers hybridized to the RMO/JI104 probe. PCR products with the RDEG primer set did not hybridize to the RDEG/KR1 probe but did hybridize to the RMO/JI104 probe when the stringency was reduced to approximately 60%. Other putative oxygenase genes were detected inconsistently in the o-xylene microcosms. PHE was only observed in the second week of the o-xylene microcosm despite being detected in all unique o-xylene isolates. Faint NAH products were noted in the o-xylene microcosm, however, none of the o-xylene isolates harbored a detectable naphthalene dioxygenase and the observed NAH products did not hybridize to the NAH/G7 probe under low stringency conditions.

TABLE 9
Aromatic Oxygenase Genes Detected in o-xylene Experiments.

| | | Primer Set | | | | | |
|---|---|---|---|---|---|---|---|
| | Total | | RMO/ | | | | |
| | Isolates | PHE | RDEG | TOL | NAH | BPH4 | TOD |
| o-xylene | 1 | 1 | 1 | 0 | 0 | 0 | 0 |
| isolates | | | | | | | |
| o-xylene | | — | ++ | — | * | — | — |
| microcosm | | | | | | | |

(++) Product was observed during all four weeks and hybridized to positive control probe.
(*) Product of the correct size was observed in at least three weeks but did not hybridize to positive control probe.
(—) No product observed.

m-Xylene and p-Xylene Experiments

Enrichment with m-xylene and p-xylene strongly selected for the TOL genotype. TOL products were observed in all m-xylene and p-xylene isolates and all four weeks of both microcosms (Table 10). Although they were detected in m-xylene isolates, faint RMO and PHE products were only observed in the second and fourth weeks of the m-xylene microcosm respectively. PHE was observed during the first three weeks of the p-xylene microcosm but not in the m-xylene microcosm. As with some benzene and toluene isolates, non-specific products which did not hybridize to the RMO/JI104 probe were observed with RMO primers and several m-xylene isolates.

TABLE 10
Aromatic Oxygenase Genes Detected in m- and p-xylene Experiments.

| | | Primer Set | | | | | |
|---|---|---|---|---|---|---|---|
| | Total | | RMO/ | | | | |
| | Isolates | PHE | RDEG | TOL | NAH | BPH4 | TOD |
| m-xylene | 9 | 1 | 4 | 9 | 0 | 0 | 0 |
| isolates | | | | | | | |
| m-xylene | | — | — | ++ | — | — | — |
| microcosm | | | | | | | |
| p-xylene | 3 | 1 | 0 | 3 | 0 | 0 | 0 |
| isolates | | | | | | | |
| p-xylene | | + | — | ++ | — | — | — |
| microcosm | | | | | | | |

(++) Product was observed during all four weeks and hybridized to positive control probe.
(+) Product was observed during three weeks and hybridized to positive control probe.
(—) No product observed.

Naphthalene and Phenanthrene Experiments

PCR products corresponding to the NAH subfamily of naphthalene dioxygenase genes were observed in all thirteen of the naphthalene-utilizing strains and throughout the enrichment period (Table 11). NAH was not observed in an anaerobic naphthalene microcosm demonstrating that oxygen was required to select for the NAH genotype. Although not present in any of the naphthalene isolates, PHE was also detected in all four weeks of the naphthalene microcosm. Non-specific products were observed with three isolates following PCR with RMO primers, but none were observed with soil DNA. No aromatic oxygenases were detected in the phenanthrene microcosm.

TABLE 11
Aromatic Oxygenase Genes Detected in Naphthalene Experiments.

| | | Primer Set | | | | | |
|---|---|---|---|---|---|---|---|
| | Total | | RMO/ | | | | |
| | Isolates | PHE | RDEG | TOL | NAH | BPH4 | TOD |
| naphthalene | 8 | 0 | 0 | 0 | 8 | 0 | 0 |
| isolates | | | | | | | |
| naphthalene | | ++ | — | — | ++ | — | — |
| microcosm | | | | | | | |

(++) Product was observed during all four weeks and hybridized to positive control probe.
(—) No product observed.

Biphenyl Experiments

BPH4 was observed in all biphenyl-utilizing isolates (Table 12), but was detected only in one biphenyl microcosm soil sample (week 3). Interestingly, biphenyl dioxygenase genes from the BPH1 and BPH2 subfamilies were not observed in any biphenyl utilizing isolates or microcosm samples. A PHE product that hybridized to the PHE/CF600 probe was also observed in two of eight biphenyl isolates. Amplification products matching the size of the PHE product were detected in the first three weeks of the biphenyl microcosm, however, none of the observed products hybridized to the PHE/CF600 probe.

TABLE 12
Aromatic Oxygenase Genes Detected in Biphenyl Experiments.

| | | Primer Set | | | | | |
|---|---|---|---|---|---|---|---|
| | Total | | RMO/ | | | | |
| | Isolates | PHE | RDEG | TOL | NAH | BPH4 | TOD |
| biphenyl | 8 | 2 | 0 | 0 | 0 | 8 | 0 |
| isolates | | | | | | | |
| biphenyl | | * | — | — | — | — | — |
| microcosm | | | | | | | |

(*) Product of the correct size was observed in at least three weeks but did not hybridize to positive control probe.
(—) No product observed.

Gasoline Experiments

PHE and TOL products were observed during the last three weeks of the gasoline microcosm and in six and three of eleven gasoline isolates, respectively (Table 13). RMO was detected throughout the enrichment period but was not observed with any of the gasoline isolates. Three of the gasoline isolates did not contain any oxygenases detected by the PCR assay used, however, no attempt was made to determine if these organisms grew on aromatic or other gasoline constituents.

TABLE 13
Aromatic Oxygenase Genes Detected in Gasoline Experiments.

| | | Primer Set | | | | | |
|---|---|---|---|---|---|---|---|
| | Total | | RMO/ | | | | |
| | Isolates | PHE | RDEG | TOL | NAH | BPH4 | TOD |
| gasoline | 11 | 6 | 0 | 3 | 0 | 0 | 0 |
| isolates | | | | | | | |
| gasoline | | + | ++ | ++ | — | — | — |
| microcosm | | | | | | | |

(++) Product as observed during all four weeks and hybridized to positive control probe.
(+) Product was observed during three weeks and hybridized to positive control probe.
(—) No product observed.

Community Analysis of Microcosm Soil DNA by PCR-DGGE and Cultivation

The effect of each enrichment substrate on the bacterial community was assessed by colony counts and PCR-DGGE. Overall, colony counts revealed that aromatic-degrading populations numbered on the order of 10⁶ cfU ml⁻¹ prior to the start of the enrichment period and either remained fairly constant or dropped to 10⁵ except in the naphthalene microcosm. Naphthalene-degraders increased from 4.6 (0.6)×10⁶ to 9.1 (1.4)×10⁷ by week four. In the unamended microcosm and those containing low BTX concentration (e.g. carbon flux <10 μg l⁻¹), little selection was apparent based on PCR-DGGE profiles of the bacterial community. With higher concentrations and solid substrates, an enrichment effect on the community structure was readily apparent.

To link changes in community structure to function, the PCR-DGGE profile of each soil microcosm was compared to 16S rDNA PCR products of environmental isolates. Several enriched dominant bands were observed in the naphthalene microcosm. The 16S rDNA products from the DNN 3-0-2 and ENN3-1-12 isolates co-migrated with a major band. Based on REP-PCR results these strains are unique yet happen to co-migrate. More importantly, both isolates harbor naphthalene dioxygenases suggesting that enrichment with naphthalene will select for the bacteria containing the NAH subfamily of naphthalene dioxygenase genes and appear to be a significant fraction of the community based on PCR-DGGE results.

As with the naphthalene microcosm, many bands were apparent following enrichment with biphenyl. The 16S rDNA products from only two isolates co-migrated with any major bands. Both of these strains harbored a BPH4-type biphenyl dioxygenase, and one contained a phenol hydroxylase gene suggesting selection of these genotypes in the biphenyl microcosm. Week 3 was chosen for comparison because the enrichment effect was less noticeable in week 4.

Enrichment with m-xylene did not have a dramatic impact on the bacterial community structure, but some selection was apparent. None of the 16S rDNA products with m-xylene isolates matched dominant bands observed during m-xylene enrichment, but products from two isolates co-migrated with distinct bands. Both of these isolates contained toluene monooxygenase by PCR amplification with TOL. In the microcosm with high toluene concentrations, several dominant bands were observed. The 16S rDNA PCR product from toluene isolate (ETT 3-1-11) co-migrated with a dominant band, however, the strain did not harbor a detectable oxygenase gene.

Comparison of REP-PCR Screening with Oxygenase Screening and Community Analysis

Overall, the results from REP-PCR screening of environmental isolates and PCR-DGGE profiles of the soil microcosms show significant shifts in the microbial community structure. In general, three or four unique strains based on REP-PCR patterns were isolated directly or following one week of enrichment. Specifically, 3 unique toluene strains, 4 m-xylene strains, and 4 naphthalene strains were initially isolated from their respective microcosms. Despite the consistent emergence of bright bands in the PCR-DGGE profiles, REP-PCR screening of environmental isolates often seemed to indicate dynamic population shifts from week to week. For example, 3 unique strains were isolated after the second week of enrichment with naphthalene and 5 unique strains were isolated form the m-xylene microcosm after the final week of incubation. Examining the number of strains isolated that belonged to each REP-PCR group in some cases however, suggested enrichment of certain REP-PCR groups. Thirteen of the nineteen pure cultures isolated from the toluene microcosm after the second week were siblings. DGGE profiles also suggested selection of these strains. All twelve of the pure cultures isolated from the o-xylene microcosm after four weeks of incubation were siblings. After the four weeks, nine of the fourteen pure cultures isolated from the m-xylene microcosm had the same REP-PCR pattern. PCR-DGGE profiles of this isolate also indicated that it was enriched under the microcosm conditions.

Detection of a particular phylogenetic group, however, would not necessarily correspond to a catabolic genotype. For example PHE was detected in only one of two toluene-utilizing pure cultures considered siblings by REP-PCR patterns. Furthermore the DGGE profiles of isolates often did not correspond to major bands in the microcosm DGGE profiles suggesting that the apparent selection of a single phylgenetic group based on REP-PCR was partially due to cultivation bias. As evidenced by PCR-DGGE profiles a diverse bacterial community is still present even after enrichment with an individual aromatic hydrocarbon indicating that even cultivation independent methods of detecting specific phylogenetic groups (such as detecting specific 16S rDNA sequences) would be inadequate for assessing catabolic potential. REP-PCR results suggest significant shifts in population from week to week. In the naphthalene microcosm for example, five of the seven cultures isolated following the second week had similar REP-PCR patterns suggesting selection of this group. Following the third week, all of the pure cultures produced a REP-PCR pattern not observed previously suggesting selection of a different group. The amplified V3 region of the rRNA gene from this strain, however, did not co-migrate with any dominant bands in the DGGE profile of the naphthalene microcosm. Interestingly, the isolates corresponding to major bands were isolated directly or after the first week of enrichment. NAH was detected in all naphthalene-utilizing isolates and in the soil DNA extracts, however, indicating that although different members of the community appeared to be selected at different times all harbored naphthalene dioxygenase genes. Overall the results from REP-PCR screening and PCR-DGGE profiles show that different phylogenetic groups dominated the microbial community structure in each microcosm at different times. Furthermore, pure cultures considered siblings did not always contain the same oxygenase genes. Therefore, approaches focusing on detection of a particular phylogenetic indicator will not be a good indicator of aromatic hydrocarbon biodegradation potential. Aromatic oxygenase genes corresponding to the enrichment substrate were consistently detected despite changes in community structure, however. Thus detection of functional genes (i.e. oxygenase genes) is needed to adequately assess biodegradation potential.

Discussion

Example 1 described the development and testing of aromatic oxygenase-specific PCR primers and protocols with well-characterized bacterial strains. In the experiments reported in Example 2, detection of aromatic oxygenase genes was combined with PCR-DGGE to gain insight into the selective pressure exerted on microbial communities by aromatic hydrocarbon contamination and to evaluate the aromatic oxygenase-specific primers with environmental isolates and total soil DNA prior to field application. When substrates were provided at high concentrations or fluxes a significant enrichment effect was observed in PCR-DGGE profiles. Low substrate concentrations or fluxes did not have a dramatic effect on the community structure during the time allowed. Regardless of whether an effect on the community structure was apparent from DGGE profiles, definite shifts were observed in terms of aromatic oxygenase genes. For all aerobic substrate amended microcosms except phenanthrene microcosms, an aromatic oxygenase corresponding to the enrichment substrate was detected indicating (1) aromatic oxygenase specific primers permitted amplification of uncharacterized isolates and target genes in soil systems and (2) PCR amplification of aromatic oxygenase genes was more sensitive than PCR-DGGE for detecting changes in degrader-populations. Furthermore, oxygenase screening of isolates with 16S rDNA products co-migrating with major bands in PCR-DGGE profiles of the naphthalene and biphenyl microcosms demonstrated that several dominant populations in these microcosms contained aromatic oxygenase genes.

Although major population shifts in community structure were not observed with benzene and toluene (low) microcosms, definite shifts were observed in terms of catabolic genes. Detection of PHE in benzene and toluene isolates as well as both microcosms indicates that enrichment with benzene or toluene selected for this genotype (Table 8). PCR products with the size predicted for RMO suggest that genes possibly related to ring hydroxylating monooxygenase genes may have also been selected. In addition, PCR products indicative of naphthalene dioxygenase (NAH) genes were detected in the benzene microcosm. The naphthalene catabolic pathway is known to be induced by the mono-aromatic intermediate salicylate. Plus, it has been reported that naphthalene dioxygenase can catalyze monooxygenation reactions with ethylbenzene, toluene, xylenes, and nitrotoluenes. Induction by benzene or a metabolite combined with the broad specificity of the naphthalene pathway could result in growth of NAH-harboring bacteria on benzene. Alternatively, the observed NAH product could result from enrichment of dioxygenase genes similar to 2,4-dinitrotoluene dioxygenase from Burkholderia sp. DNT targeted by the NAH primers.

Presumably due to the low carbon flux used, no major changes in community structure were observed in the o-xylene microcosm. Enrichment with o-xylene, however, did select for the RMO genotype (Table 9). Results with o-xylene isolates suggested PHE was also selected but the phenol hydroxylase product was only observed during week two. Phenol hydroxylases are responsible for further oxidation of o-xylene intermediates and were expected to be observed more consistently. As with the benzene microcosm, a putative naphthalene dioxygenase was also detected in the o-xylene microcosm.

A slight enrichment effect was observed in the DGGE profiles of the m-xylene microcosm. Although species representative of major bands were not isolated from the m-xylene microcosm, the 16S rDNA PCR product from TOL-harboring strains did co-migrate with some distinct bands in the microcosm DGGE profile suggesting a role for TOL in the metabolism of m-xylene in the environment. Furthermore, TOL was detected consistently during the genotype screening of the m-xylene microcosm and all m-xylene isolates (Table 10). In the p-xylene microcosm, little selection was noticeable in DGGE profiles likely due to the low flux. Both TOL and PHE were detected in the p-xylene microcosm and isolates, however, indicating that they are involved in biodegradation of this compound.

PCR-DGGE profiles of the gasoline microcosm did not reveal major shifts in the bacterial community structure, but again oxygenase genes were detected following enrichment (Table 13). PHE and TOL were detected in gasoline isolates and microcosm samples. Comparison with the pure compound microcosm results suggests that the benzene, toluene, and p-xylene fractions of gasoline selected for PHE and that the m-xylene and p-xylene fractions selected for TOL. Enrichment by o-xylene and possibly benzene and toluene likely led to detection of RMO in the gasoline microcosm.

In the naphthalene microcosm PCR-DGGE profile, selection of dominant bands was readily apparent. Again corresponding strains were not isolated for each band, but the two that did correspond to major bands harbored naphthalene dioxygenase genes indicating enrichment of species harboring the subfamily of naphthalene dioxygenase genes targeted by the NAH primer set. NAH was not detected in the anaerobic naphthalene microcosm suggesting that oxygen is required for the selection of bacteria which utilize oxygenases as part of aromatic hydrocarbon catabolism by aerobic pathways. PHE was detected throughout the naphthalene enrichment indicating selection of PHE-harboring bacteria, but none of the naphthalene isolates contained PHE (Table 11). As evidenced by the DGGE analysis of the naphthalene microcosm, the library of naphthalene isolates was not representative of the entire bacterial community. The strains containing PHE may have been a portion of the unculturable population. Strains whose V3 amplification products co-migrated with two of the dominant bands in biphenyl microcosm profiles were isolated. Both contained BPH4 suggesting the importance of this subfamily of biphenyl dioxygenase in biphenyl catabolism. The DGGE profile of the biphenyl microcosm had less dominant bands in week 4 than in week 3, suggesting a decrease in biphenyl-utilizing bacteria. Furthermore, PHE was detected in each of the first three weeks but not in the fourth which may mean that something had an adverse effect on the overall community. Perhaps BPH4 would have been detected in week 4 if the microcosm had remained “healthy”, however, the fact that BPH4 was only detected once during the enrichment period suggests uncharacterized biphenyl dioxygenases or pathways may be primarily responsible for biphenyl biodegradation.

Even though they were spiked with individual substrates, at least two different oxygenase gene subfamilies were detected in each microcosm. The genotype screening of the environmental isolates was further examined to determine if co-occurring oxygenase genes were the result of different members of the community or whether individual bacteria often harbor multiple aromatic oxygenase genes. A single type of aromatic oxygenase was detected in the majority of the isolates suggesting the former, however, twelve of the fifty-four pure compound isolates contained two types of oxygenase genes indicating that the latter is not rare (Table 14).

TABLE 14
Co-occurring Aromatic Oxygenase Genes.

| Environmental | Primer Set | | | | | Isolation |
|---|---|---|---|---|---|---|
| Isolate | PHE | NAH | TOL | RMO/RDEG | BPH4 | Substrate |
| o-x-1 | + | — | — | + | — | o-xylene |
| o-x-4 | + | — | — | + | — | o-xylene |
| o-x-7 | + | — | — | + | — | o-xylene |
| o-x-9 | + | — | — | + | — | o-xylene |
| EXX 4-1-14 | + | — | + | — | — | m-xylene |
| p-x-11 | + | — | + | — | — | p-xylene |
| EGG 3-1-13 | + | — | + | — | — | gasoline |
| BPH1 | + | — | — | — | + | biphenyl |
| BPH3 | + | — | — | — | + | biphenyl |
| EXX 3-1-11 | — | — | + | + | — | m-xylene |
| EXX 4-4-25 | — | — | + | + | — | m-xylene |
| EXX 4-4-35 | — | — | + | + | — | m-xylene |
| EXX 4-4-36 | — | — | + | + | — | m-xylene |

(+) denotes that the described product was observed and hybridized to appropriate probe.
(—) oxygenase product was not observed.

In nine of these cases PHE was one of the oxygenase genes detected. All of the o-xylene isolates harbored PHE and RMO. The catabolic pathway for o-xylene often involves initial oxidation by a ring-hydroxylating monooxygenase followed by further oxidation mediated by a phenol hydroxylase so the co-occurrence of these two genotypes is logical. Detection of PHE in TOL-harboring isolates and BPH4-harboring isolates is more puzzling. Perhaps the co-occurrence of these genotypes is not linked but PHE could be involved in downstream metabolism of intermediates like benzoate. TOL and RMO were both detected in 4 of 9 m-xylene isolates. The advantage of this type of functional redundancy remains unclear, however, multiple pathways within a strain for the catabolism of toluene has been previously reported.

One of the objectives of the microcosm study was to determine the effect of aromatic hydrocarbon contamination on the indigenous bacterial community. Overall, no major population shifts were observed in DGGE profiles when low carbon fluxes (<10 g carbon g soil⁻¹ week⁻¹) were supplied. Conversely, high carbon fluxes selected for multiple dominant species in each microcosm.

In the low toluene flux microcosm very little selection is apparent after the first week whereas the high flux microcosm shows enrichment. Assuming 10% conversion of substrate carbon to biomass (44% carbon by weight and 10⁻¹³ g cell⁻¹) production would amount to approximately 1.25×10⁷ and 3.7×10⁸ cells g soil⁻¹ week⁻¹. These growth rates correspond to 0.1% and nearly 4% of the bacterial population (10¹⁰ cells g soil⁻¹). Prior estimates indicate that a given 16S rDNA sequence type must comprise at least 1% of the total target organisms to be discernable from background amplification products. Thus changes in community structure in low flux microcosms were often undetectable. PCR amplification of aromatic oxygenase genes in these microcosms compared to the unamended control, however, showed functional changes in the population. In terms of site remediation, the low BTX concentrations used in the microcosms is more representative of field conditions. Therefore, tracking changes in the community structure by PCR-DGGE is not always sufficient to address the impact of contamination on the microbial population.

The microcosm study was also used to evaluate the aromatic oxygenase-specific primers with environmental isolates and total DNA from soil samples prior to use at petroleum-contaminated sites. In the present study, no oxygenase genes were consistentyl detected in the samples from the unamended and anaerobic microcosms, however, aromatic catabolic oxygenase genes were detected in virtually all enrichment microcosms and environmental isolates (Tables 15 and 16).

TABLE 15
Summary of Genotype Screening of Environmental Isolates.

| | Total | | | | | | | |
|---|---|---|---|---|---|---|---|---|
| | Cul- | Growth | | RMO/ | | | | |
| | tures | Substrate | PHE | RDEG | TOL | NAH | BPH4 | TOD |
| | 5 | benzene | 3 | 1 | | | 1 | |
| | 6 | toluene | 2 | 1 | | | | |
| | 1 | o-xylene | 1 | 1 | | | | |
| | 9 | m-xylene | 1 | 4 | 9 | | | |
| | 3 | p-xylene | 1 | | 3 | | | |
| | 8 | naph- | | | | 8 | | |
| | | thalene | | | | | | |
| | 8 | biphenyl | 2 | | | | 8 | |
| Sum | 40 | | 10 | 7 | 12 | 8 | 9 | 0 |

TABLE 16
Summary of Genotype Screening of Microcosms.

| | Primer Set | | | | | | | |
|---|---|---|---|---|---|---|---|---|
| | PHE | RMO/RDEG | TOL | TOD | NAH | BPH1 | BPH2 | BPH4 |
| Microcosm | | | | | | | | |
| benzene | + | * | − | − | + | − | − | − |
| toluene (low) | ++ | * | − | − | − | − | − | − |
| toluene | ++ | − | − | − | − | − | − | − |
| (high) | | | | | | | | |
| o-xylene | − | ++ | − | − | * | − | − | − |
| m-xylene | − | − | ++ | − | − | − | − | − |
| p-xylene | + | − | ++ | − | − | − | − | − |
| naphthalene | ++ | − | − | − | ++ | − | − | − |
| biphenyl | * | − | − | − | − | − | − | − |
| phenanthrene | − | − | − | − | − | − | − | − |
| gasoline | + | ++ | ++ | − | − | − | − | − |
| unamended | − | − | − | − | − | − | − | − |

(++) Product was observed during all four weeks and hybridized to positive control probe.
(+) Product was observed during three weeks and hybridized to positive control probe.
(*) Product of the correct size was observed in at least three weeks but did not hybridize to positive control probe.
(−) No product observed.

PHE, RMO, and TOL were all detected in the gasoline microcosm and therefore will likely be good indicators of bioremediation potential at gasoline-contaminated sites. The PHE primer set may be a particularly important indicator. First, PHE was consistently detected in the benzene microcosm even when low substrate concentrations were maintained. Considering the high solubility, high toxicity, and corresponding low maximum contaminant level (MCL) for benzene, the ability to detect a catabolic genotype involved in benzene biodegradation is critical for field applications. Second, PHE was detected in naphthalene and biphenyl microcosm samples and along with NAH may be a good indicator of biodegradation potential at diesel- or PAH-contaminated sites. TOL was detected only in the m-xylene and p-xylene microcosms indicating that TOL may be more involved in the catabolism of these xylene isomers than toluene. Thus, the m-xylene and p-xylene fractions of gasoline may have enriched for TOL-harboring bacteria in the gasoline microcosm. Interestingly, TOD was not detected in any isolates or microcosm samples. While detection of naphthalene dioxygenase genes in environmental samples has been documented by several groups, reports on detection of toluene dioxygenase differ. One group recently estimated that the todC1C2 containing fraction of the microbial community at a petroleum-contaminated aquifer was greater than the tomA (RMO) and xylA (TOL) fractions, whereas other groups did not detect toluene dioxygenase in environmental samples and isolates in which a TOL plasmid was detected.

The microcosm study was used to evaluate the oxygenase-specific PCR primers and provide insight into the selection of aromatic catabolic pathways to aid in interpretation of results from gasoline-contaminated sites. Of the fifty-four strains isolated on pure compounds, fifty-one contained at least one detectable oxygenase corresponding to the growth substrate. Furthermore, oxygenase genes corresponding to the enrichment substrate were detected in all aerobic microcosms supplied with an aromatic hydrocarbon. No oxygenase genes were detected in the anaerobic and unamended microcosms demonstrating that the aromatic substrate and oxygen are required for selection of aerobic aromatic-degraders. This result will be particularly useful for evaluating results from field sites which may have anaerobic zones. PHE, RMO, and TOL were all detected in the gasoline microcosms and will therefore likely be good indicators of bioremediation potential at gasoline-contaminated sites. NAH was also detected in the benzene microcosm indicating it may also be detected at gasoline sites. Comparison of 16S rDNA PCR products from naphthalene isolates with the DGGE profile of the naphthalene microcosm confirmed that NAH-harboring strains were among the dominant species suggesting NAH will also be used at sites contaminated by PAHs. Enrichment of aromatic catabolic genotypes in amended microcosms was noted even when changes in community structure were not always evident and strains representative of dominant species could not isolated. While this means a conclusive link between the observed change in catabolic genes and changes in community structure could not always be established, the results clearly demonstrate the need to target functional genes to evaluate microbial communities for site remediation purposes.

### Example Three

In this study, we utilized an array of primers and a real-time PCR protocol described in Example 1 to detect and enumerate aromatic oxygenase genes at two gasoline-contaminated sites currently undergoing monitored natural attenuation. Aromatic oxygenase genes were chosen as indicators because they play a key role in the biodegradation of BTEX, the pollutants of principal concern at gasoline-contaminated sites. From the microcosm results (Table 17), PHE, RMO, and TOL were expected to be observed at the gasoline-contaminated sites. Furthermore, enrichment of naphthalene in the benzene microcosm and the presence of biphenyl dioxygenase in a benzene-utilizing isolate suggested that these oxygenase genes might also be observed.

TABLE 17
Summary of Microcosm and Isolate Results.

| Pure Culture and | | | | | |
|---|---|---|---|---|---|
| Microcosm | PHE | RMO/RDEG | TOL | NAH | BPH4 |
| benzene | ++ | *+ | − | + | X |
| toluene (low) | ++ | * | − | − | − |
| toluene (high) | ++ | − | − | − | − |
| o-xylene | X | ++ | − | * | − |
| m-xylene | X | X | ++ | − | − |
| p-xylene | ++ | − | ++ | − | − |
| naphthalene | ++ | − | − | ++ | − |
| biphenyl | *+ | − | − | − | X |
| gasoline | ++ | ++ | ++ | − | − |

(++) Product was observed during at least 3 weeks of the microcosm and in isolates grown on the substrate. Products hybridized to positive control probe.
(+) Product was observed during three weeks and hybridized to positive control probe. Products were not detected in isolates.
(*+) Product of the correct size was observed in at least three weeks but did not hybridize to positive control probe. Products observed with isolates hybridized to probe.
(*) Products observed in microcosm which did not hybridize to the probe. Product not detected in isolates.
(X) The genotype was observed in isolates but not consistently detected in the microcosm.
(−) No product observed.

DGGE analysis, used to document the effect of hydrocarbon contamination on the microbial community at each site, revealed that subpopulations of microbial communities were enriched in contaminated areas. Comparison of BTX data and copy numbers of aromatic oxygenase genes indicated that both sites maintain BTX-degrading communities within and downgradient of impacted zones.

Materials and Methods

Field Site History

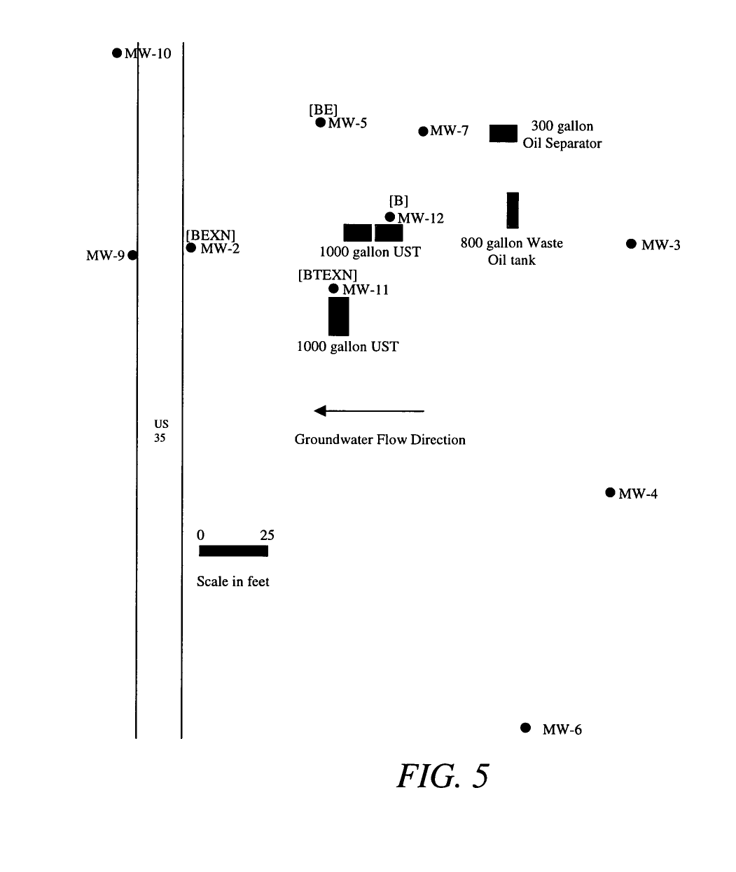

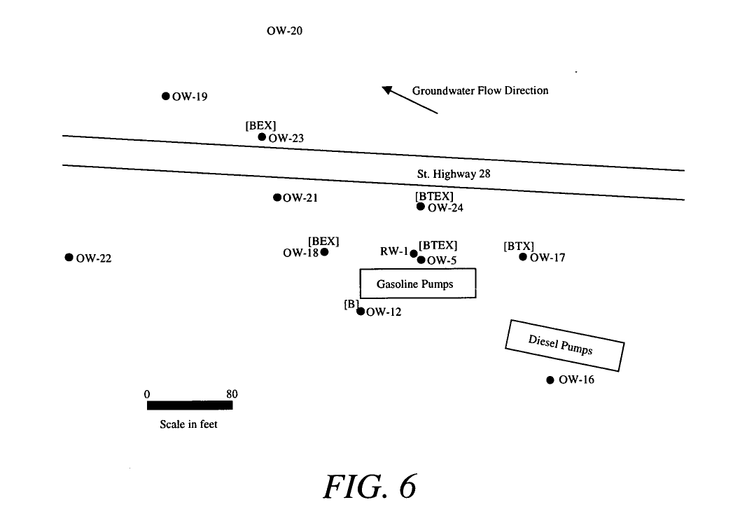

Groundwater samples were collected from gasoline-contaminated sites in Winamac and Frankfort, Ind. Prior to 1996, diesel fuel, gasoline, heating oil, and waste oil underground storage tanks (USTs) were removed from the Winamac site (FIG. 5) as described by Mesarch et al. (Mesarch, M. B., C. H. Nakatsu, and L. Nies. 2000. Development of catechol 2,3-dioxygenase-specific primers for monitoring bioremediation by competitive quantitative PCR. Applied and Environmental Microbiology 66:678-683.). The Frankfort site is an operating gasoline and diesel fuel station (FIG. 6). Beneath the pavement, the gravel subbase is underlain by discontinuous brown poorly graded gravels and fine to medium grained sands extending from 2 to 5 feet below the surface. A brown continuous silty clay extends from 5 to 15 feet below the surface which is underlain by a brown clayey fine to medium grained sand which extends to the bottom of RW-1 at 30 feet. Groundwater flows toward the northwest.

Groundwater Sampling and Processing

Groundwater samples were taken from Winamac in April, 2000 and Frankfort in September, 2001 using sterile disposable bailers. Wells at the Winamac site were purged prior to sampling and 40 ml samples were sent on ice to certified laboratories for BTX and SVOC analysis by established USEPA methods GC(601/602, 8010/8020). Wells were not purged prior to sampling at the Frankfort site. For both sites, one liter groundwater samples were collected in sterile glass bottles and stored on ice before being taken to Purdue University for genetic analysis. Within 12 hours of sampling, solids from groundwater samples were collected by centrifugation at 10,000×g for 30 minutes.

DNA Extraction and Multiplex PCR

DNA extractions were performed with 0.5 g of aquifer solids using the FastPrep Soil DNA extraction kit (BIO101, Vista, Calif.) and the FP120 FastPrep Cell Disruptor (Savant Instruments Inc., Holbrook, N.Y.). Samples were initially screened for aromatic oxygenase genes using the PCR primers and multiplex PCR protocols described in Example 1. All PCR experiments included reactions with DNA extracts from appropriate positive control strains and reactions containing no template. PCR products were routinely visualized by running 10 μL of PCR mixture on 1% agarose gels (Bio-Rad, Richmond, Calif.) in 1× Tris-Acetate-EDTA (TAE) buffer stained with ethidium bromide (0.0001%). For all samples containing putative oxygenase genes, the PCR products were separated on an agarose gel, transferred to a nylon membrane, and hybridized under low stringency conditions as described in Example 1.

DNA Quantification

DNA concentrations of positive control strains were quantified by fluorometry using a Model TKO100 DNA Fluorometer (Hoefer Scientific Instruments, San Francisco, Calif.) calibrated with calf thymus DNA. Standards ranging from 10⁶ to 10² copies rxn⁻¹ for real-time PCR were made from serial dilutions of DNA extracts from positive control strains.

Real-time Q-PCR with SYBR Green I

Aromatic oxygenase gene copy numbers were determined by real-time PCR for all positive samples during initial screening on agarose gels as described in Example 1. Calibration curves for each target were made with standards during each real-time PCR experiment. One× and 1/10× dilutions of all environmental samples were analyzed in duplicate.

Real-time PCR was performed on an ABI 7700 Sequence Detector with Sequence Detector (version 1.7) software (Applied Biosystems, Foster City, Calif.) as described in Example 1. The program subtracted the background signal for each sample determined in cycles 3 through 15. The fluorescence threshold was defined as ten times the standard deviation of the background signal. The threshold cycle was defined as the fractional cycle number in which the signal exceeded the fluorescence threshold. The computed threshold cycle inversely correlated to the log of the initial template concentration. Some false positives occurred with environmental samples in which a threshold cycle was computed with no increase in fluorescence signal. In such cases these cycle numbers were not used to calculate copies g⁻¹ soil.

DGGE Analysis of Community Structure

PCR-DGGE analysis of the microbial community structure was performed as described in Example 2 for soil microcosms.

Results

BTX Concentrations at Winamac Site

At the time of sampling, five monitoring wells at the Winamac site contained detectable BTX concentrations (FIG. 5; Table 18).

TABLE 18
BTEX Concentrations at Winamac Site
BTEX Concentrations at the Winamac Site.

| Well | Benzene | Toluene | Ethylbenzene | Xylenes |
|---|---|---|---|---|
| Sample | (μg l⁻¹) | (μg l⁻¹) | (μg l⁻¹) | (μg l⁻¹) |
| MW-2 | 61 | ND | 8.3 | 83 |
| MW-3 | ND | ND | ND | ND |
| MW-4 | ND | ND | ND | ND |
| MW-5 | 290 | ND | 5.8 | ND |
| MW-6 | ND | ND | ND | ND |
| MW-7 | ND | ND | ND | ND |
| MW-9 | ND | ND | ND | ND |
| MW-10 | ND | ND | ND | ND |
| MW-11 | 1,300 | 1,300 | 180 | 1,100 |
| MW-12 | 26 | ND | ND | ND |

ND—Not detected

In addition, naphthalene was found in MW-2, MW-8, and MW-11 at concentrations of 21, 14, and 47 μg l⁻¹, respectively. Although not detected during the most recent sampling, xylenes were detected in MW-3 and MW-7 in 1996 and 1998, respectively. MW-4, MW-6, and MW-10 are located outside the plume and have had no history of contamination.

BTX Concentrations at Frankfort Site

At the Frankfort site, total BTEX concentrations greater than 50 mg l⁻¹ (Table 19) were detected in the vicinity of the gasoline pump islands (FIG. 6; RW-1, OW-5, and OW-24). Monitoring wells surrounding this area also had elevated BTEX levels (OW-12, OW-1 7, OW-18, OW-23). BTEX was not detected in OW-16 located upgradient nor monitoring wells OW-19, OW-20, and OW-22 located downgradient.

TABLE 19
BTEX Concentrations at Frankfort Site
BTEX Concentrations at the Frankfort Site.

| Well | Benzene | Toluene | Ethylbenzene | Xylenes |
|---|---|---|---|---|
| Sample | (μg l⁻¹) | (μg l⁻¹) | (μg l⁻¹) | (μg l⁻¹) |
| RW-1 | 20,000 | 11,000 | 1,600 | 2,200 |
| OW-5 | 24,000 | 38,000 | 2,400 | 12,700 |
| OW-12 | 190 | ND | ND | ND |
| OW-16 | ND | ND | ND | ND |
| OW-17 | 2,700 | 5,000 | ND | 1,900 |
| OW-18 | 5,000 | ND | 980 | 760 |
| OW-19 | ND | ND | ND | ND |
| OW-20 | ND | ND | ND | ND |
| OW-21 | ND | ND | ND | ND |
| OW-22 | ND | ND | ND | ND |
| OW-23 | 75 | ND | 530 | 580 |
| OW-24 | 24,000 | 22,000 | 1,400 | 4,600 |

ND—Not detected

Bacterial Community Structure

The presence of contamination appears to have had a marked effect on the microbial community structure at both sites as evidenced by DGGE analysis. In uncontaminated wells at the Winamac site, a smear of 16S rDNA products can be observed (MW-4, MW-6, and MW-10) whereas in contaminated and previously impacted wells brighter, more distinct bands are evident (MW-2, MW-3, MW-7, MW-11, and MW-12). Distinct bands were also observed in MW-9. Despite the presence of aromatic hydrocarbons in MW-5 only faint bands were observed within the smear of amplification products.

At the Frankfort site, wells with the highest contaminant levels (RW-1, OW-5, OW-12, OW-17, OW18, OW-23, and OW-24) show multiple dominant bands while the PCR-DGGE profiles of OW-16, OW-19, OW-20 which contained non-detectable BTEX levels was a smear of products with a few faint bands. Selection of dominant bands was observed in OW-12, OW-21, and OW-22 despite non-detectable BTEX concentrations.

Detection of Aromatic Oxygenase Genes at Winamac Site

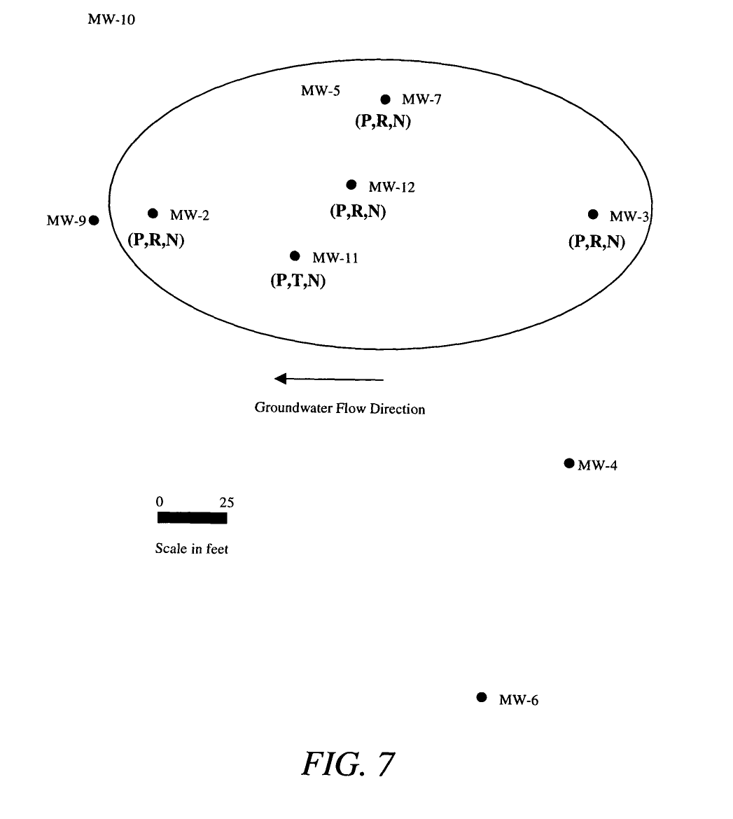

Aromatic oxygenase genes (PHE, TOL, RMO, and NAH) were detected and quantified in most BTX impacted wells from the site (FIG. 7; Table 20). No oxygenase genes were detected in any of the wells without a history of contamination. PHE, RMO, and NAH were detected in the previously contaminated monitoring wells MW-3 and MW-7 that did not contain BTX above detection limits at the time of sampling. Despite significant benzene and ethylbenzene concentrations in MW-5, no oxygenase genes were detected. In addition to MW-2 and MW-11 that contained naphthalene, NAH genes were detected in BTX contaminated wells where naphthalene was not detected. Toluene and biphenyl dioxygenases (TOD and BPH) were not detected in samples gathered from the Winamac site.

TABLE 20
Enumeration of Aromatic Oxygenase Genes at Winamac

| | PCR Primer | | | | |
|---|---|---|---|---|---|
| Well | PHE | TOL | TOD | RMO | NAH |
| MW-2 | 6.0 (4.9)E+08 | ND | ND | 1.6 (0.6)E+06 | 4.3 (1.2)E+05 |
| MW-3 | 1.4 (1.3)E+07 | ND | ND | 4.4 (1.2)E+05 | 8.7 (5.0)E+05 |
| MW-4 | ND | ND | ND | ND | ND |
| MW-5 | ND | ND | ND | ND | ND |
| MW-6 | ND | ND | ND | ND | ND |
| MW-7 | 1.4 (0.7)E+08 | ND | ND | 5.5 (2.2)E+06 | 9.6 (3.3)E+05 |
| MW-9 | ND | ND | ND | ND | ND |
| MW-10 | ND | ND | ND | ND | ND |
| MW-11 | 2.4 (2.2)E+07 | 5.3 (1.5)E+06 | ND | ND | 6.5 (2.2)E+05 |
| MW-12 | 7.8 (4.8)E+07 | ND | ND | 2.8 (2.6)E+06 | 3.0 (1.0)E+05 |

(standard deviation) of at least two replicates.

Detection of Aromatic Oxygenase Genes at Frankfort Site

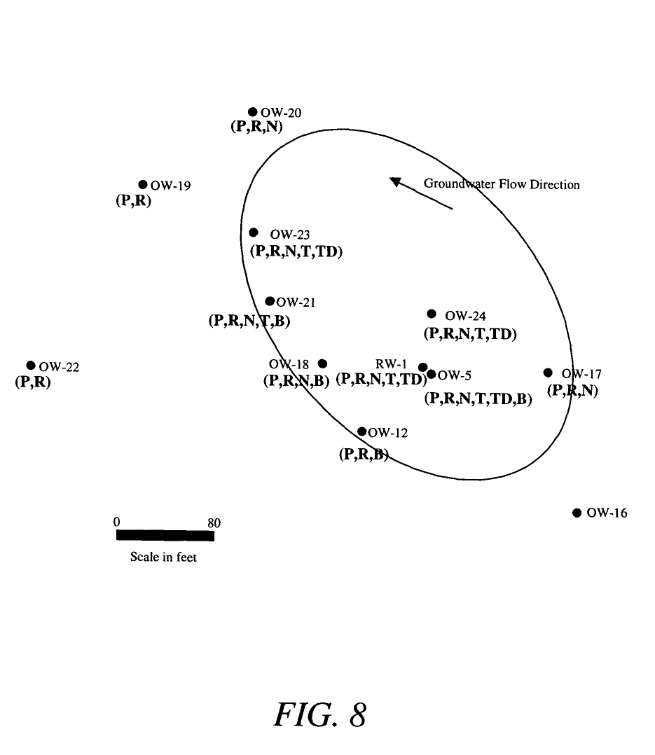

Oxygenase genes enumerated from the groundwater samples from the Frankfort site are shown in FIG. 8 and Table 21. As with the Winamac site, PHE, RMO, and NAH were detected in nearly all BTEX impacted wells. TOL was detected in wells with high BTEX concentrations within the original source area (RW-1, OW-5, and OW-24) and directly downgradient (OW-21 and OW-23). TOD was also enumerated in the center of the plume (RW-1, OW-5, OW-23, and OW-24). Near the fuel pumps, BPH4 were also detected and at times in copy numbers exceeding 10⁷ copies g⁻¹ soil (OW-5, OW-12, OW-18, and OW-21).

TABLE 21
Enumeration of Aromatic Oxygenase Genes at Frankfort Site

| | PCR Primer | | | | | |
|---|---|---|---|---|---|---|
| Well | TOL | TOD | PHE | RMO | NAH | BPH4 |
| RW-1 | 3.8 (1.0) | 2.5 (1.4) | 1.8 (1.2) | 4.3 (3.4) | 7.5 (2.4) | ND |
| | E+05 | E+07 | E+08 | E+06 | E+06 | |
| OW-5 | 2.2 (0.5) | 1.1 (0.1) | 6.2 (1.9) | 1.3 (0.1) | 1.7 (0.2) | 1.7 (0.6) |
| | E+06 | E+07 | E+08 | E+07 | E+07 | E+07 |
| OW-12 | ND | ND | 1.2 (**) | 7.3 (2.8) | ND | 7.1 (2.9) |
| | | | E+08 | E+06 | | E+06 |
| OW-16* | ND | ND | ND | ND | ND | ND |
| OW-17 | ND | ND | 6.7 (1.6) | 7.1 (1.1) | 8.3 (1.2) | ND |
| | | | E+07 | E+06 | E+06 | |
| OW-18 | ND | ND | 3.8 (1.1) | 2.3 (0.3) | 1.2 (0.3) | 2.0 (2.2) |
| | | | E+07 | E+06 | E+06 | E+06 |
| OW-19 | ND | ND | 2.8 (2.1) | 4.3 (4.7) | ND | ND |
| | | | E+08 | E+06 | | |
| OW-20 | ND | ND | 1.7 (0.8) | 8.0 (16) | 9.5 (0.9) | ND |
| | | | E+08 | E+06 | E+05 | |
| OW-21 | 2.9 (0.3) | ND | 1.7 (0.2) | 1.3 (0.8) | 5.2 (1.1) | 6.5 (3.7) |
| | E+07 | | E+09 | E+07 | E+07 | E+06 |
| OW-22 | ND | ND | 4.1 (2.2) | 2.0 (1.9) | ND | ND |
| | | | E+07 | E+06 | | |
| OW-23 | 2.1 (0.4) | 4.7 (5.7) | 2.1 (1.5) | 1.9 (1.4) | 2.9 (0.5) | ND |
| | E+07 | E+05 | E+08 | E+06 | E+06 | |
| OW-24 | 3.0 (2.9) | 1.3 (0.4) | 5.9 (1.3) | 7.4 (0.6) | 3.2 (0.5) | ND |
| | E+06 | E+07 | E+09 | E+07 | E+07 | |

(standard deviation) of at least two replicates
*No DNA was observed on an agarose gel, however, products were observed with 16S rDNA specific primers.
**Only one duplicate yielded a quantifiable product.

Enumeration of Aromatic Oxygenase Genes at Field Sites

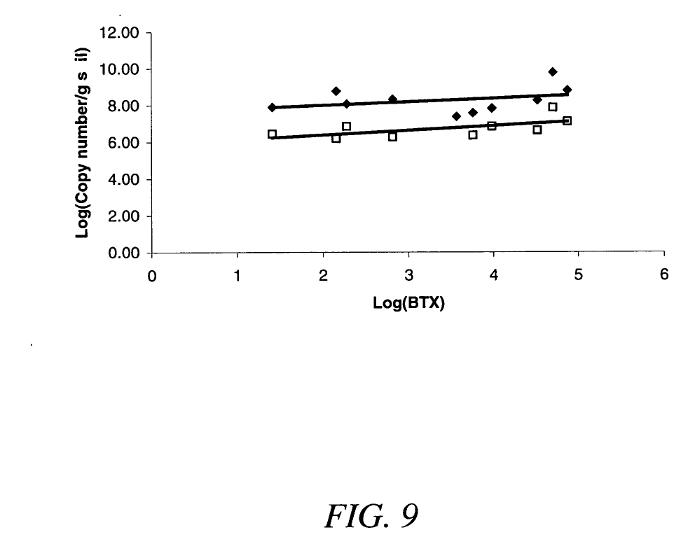

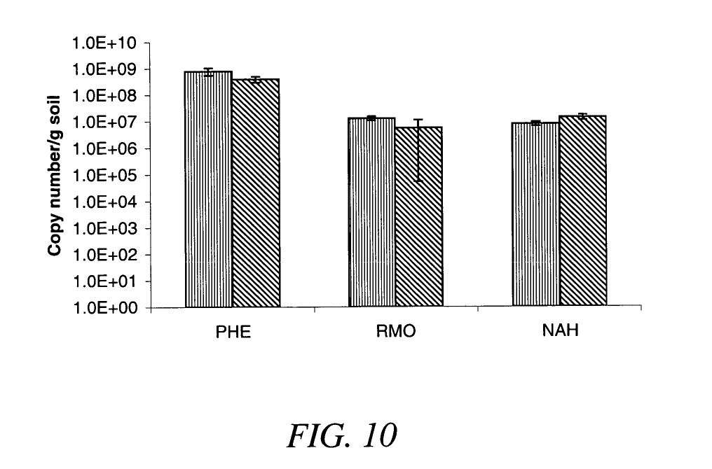

Combining the Q-PCR results from both field sites, PHE, RMO, and NAH were the most commonly detected. For all wells, however, PHE was detected in the highest copy number and usually an order of magnitude greater than other oxygenase genes. Obvious trends were not observed between BTX concentrations and aromatic oxygenase gene copy numbers individually (FIG. 9) or totaled indicating that a factor other than carbon was limiting. The strongest correlation was between NAH copy number and log(BTX), however, naphthalene concentrations were not measured for all wells. In contaminated wells, aromatic oxygenase gene copy numbers were greater than 10⁵ copies g⁻¹ whereas oxygenase genes were not detected in upgradient wells. PHE, RMO, and NAH were routinely observed in downgradient wells at the Frankfort site (OW-19, OW-20, and OW-22) despite non-detectable BTEX levels in these wells. Copy numbers for these genes in the downgradient wells was similar to those observed in the wells near the center of the plume (FIG. 10).

Discussion

At gasoline-contaminated sites, groundwater BTEX concentrations are routinely measured to track contaminant removal, but no biological assay is usually done to establish that biodegradation is the removal mechanism even when bioremediation is the prescribed corrective action. In the current study we have detected and enumerated aromatic oxygenase genes involved in the biodegradation of specific aromatic hydrocarbons at two gasoline-contaminated sites as a means to document biodegradation. The two sites represent opposite ends of the spectrum—the Winamac site is an older site nearing closure with low residual BTEX whereas the Frankfort site is an operating facility with high aqueous BTEX concentrations. At the Winamac site, high copy numbers of aromatic oxygenase genes were enumerated in impacted wells but none were detected in “sentinel wells” outside the plume. Aromatic oxygenase genes were also detected at Frankfort in the contaminated and downgradient wells but not in the upgradient well. Overall, the integration of chemical and genetic analysis gave a more clear indication of on-going remediation at the sites.

At the Winamac site, most of the currently contaminated wells are within 25 feet of the former oil separator, 8000 gallon UST, and the 1000 gallon USTs (MW-5, MW-11, MW-12, respectively). The other currently impacted well (MW-2) is less than 50 feet downgradient of the former UST locations. PHE was detected in all and RMO was detected in nearly all of the wells with detectable BTEX levels. In addition NAH was enumerated in MW-2 and MW-11 which contained naphthalene and TOL was detected in MW-11. Although these results correspond well with the chemical data, results with MW-3, MW-7, and MW-5 were at first counter-intuitive. PHE, RMO, and NAH were enumerated in MW-3 and MW-7 despite non-detectable BTEX concentrations in current samples. In prior sampling periods, however, xylenes were detected in both wells. Results from microcosm studies (Table 17) demonstrate that o-xylene and p-xylene selected for the RMO and PHE genotypes, respectively. Furthermore, the DGGE profiles of MW-3 and MW-7 samples show bright, distinct bands similar to profiles of the currently impacted wells. PCR-DGGE profiles of contaminated soils often show a shift from a complex community with a few discernable major bands in uncontaminated samples (smear of 16S rDNA products) to a reproducible pattern of major bands from samples within the contaminated area. The shifts in community structure can also correlate to contaminant concentration. Furthermore with some of the microcosms discussed in Example 2, major bands represented species capable of growth on the aromatic substrate. Thus selection of aromatic hydrocarbon-degraders and detection of significant numbers of aromatic oxygenase genes in these wells is reasonable. Oxygenase genes were not observed in MW-5 samples although benzene and ethylbenzene were detected at the time of sampling. The DGGE profile of MW-5 samples showed little selection of dominant species similar to those of uncontaminated wells. MW-5 is near the waste oil separator roughly in the middle of the plume; it has been reported that the centers of contaminant plumes are often anerobic because oxygen uptake rates exceed recharge rates.

The Frankfort site is representative of a gasoline-contaminated site at the early stages of remediation. In RW-1, OW-5, and OW-24 near the source, groundwater BTEX concentrations are high (>30 mg l⁻¹ total BTEX) which apparently led to selection of many subfamilies of aromatic oxygenase genes. PHE, RMO, NAH, TOL, TOD, and BPH4 were enumerated in samples taken from the center of the plume. Moving farther from the pump islands, groundwater BTEX levels, particularly toluene, tend to decrease (OW-12, OW-17, OW-18, OW-21, and OW-23). Many oxygenases in terms of copy numbers and subfamilies were still observed in this zone. Despite non-detectable BTEX concentrations, OW-21 contained PHE, RMO, NAH, TOL, and BPH4-harboring microorganisms. However, OW-21 is located in between two contaminated wells (OW-18 and OW-23), contained MTBE, and is therefore likely to have been impacted. OW-19, OW-20, and OW-22 are the farthest downgradient from the source but PHE, RMO, and NAH gene copy numbers were enumerated in these wells. Although BTEX were not observed in these wells, MTBE was detected in OW-20 suggesting that the contaminant plume has migrated farther downgradient than OW-23. The presence of aromatic catabolic genotypes in these wells may indicate an aromatic hydrocarbon-degrading population is responsible for non-detectable BTEX levels, is utilizing biodegradation intermediates, or is being advectively transported from the edge of the plume. Oxygenase genes were not observed in OW-16, the only upgradient well sampled, indicating that the oxygenase genes observed in OW-19, OW-20, and OW-22 are not the result of a background population.

Naphthalene dioxygenase genes were detected in three BTX-contaminated wells at the Winamac site despite non-detectable naphthalene concentrations. NAH was also detected at the Frankfort site, however, naphthalene concentrations were not measured. Although high copy numbers of naphthalene dioxygenase genes seemed counter-intuitive, naphthalene-degrading bacteria often utilize a broad range of aromatic compounds including mono-aromatic hydrocarbons (Baldwin, B. R., M. B. Mesarch, and L. Nies. 1999. Broad substrate specificity of biphenyl- and naphthalene-utilizing bacteria. Applied Microbiolology and Biotechnology 53:748-753.). Furthermore, prior work has suggested that the naphthalene pathway will frequently co-oxidize mono-aromatics, may play a role in biodegradation of BTEX, and is induced by salicylate, a mono-aromatic intermediate. The presence of naphthalene dioxygenase genes in these wells may therefore result from BTEX-degrading populations. Biphenyl dioxygenase (BPH4) was also detected at the Frankfort site. Previous reports have noted the sequence similarity and functional overlap of biphenyl and alkyl-benzene dioxygenases including toluene dioxygenase. Detection of biphenyl dioxygenase at the Frankfort site, therefore, suggests that this genotype may be selected by aromatic-hydrocarbon mixtures like petroleum products.

Some trends can be noted when the results of the field tests are combined with the microcosm results (Table 22). PHE and RMO were detected in nearly all impacted wells and those on the edge of the contaminant plume at both sites. In the microcosm study, PHE was consistently detected in the benzene, toluene, p-xylene, naphthalene, and gasoline microcosms. RMO was detected in the o-xylene and gasoline microcosms (Table 17). In addition, it has been reported that PHE and RMO are often induced by cresol intermediates. The prevalence of these genotypes in the field may therefore stem from the abundance of the wide variety of acceptable substrates present in gasoline; thus these genotypes may be very important for biodegradation in the field. Conversely, TOL and TOD were primarily observed in well samples with high BTEX concentrations. While host strain-specific factors cannot be ignored the results suggest that ring-hydroxylating pathways for BTEX catabolism may confer a competitive advantage under certain field conditions. In one to one competition experiments, it has been shown that Pseudomonas putida mt-2, harboring a TOL plasmid, was the least competitive of the known toluene pathways under both toluene- and oxygen-limiting conditions. At higher toluene concentrations, however, the disadvantage were shown to decrease, which may explain why TOL was detected in the highly contaminated wells within the plume. Moreover, TOL was detected in the gasoline, m-xylene, and p-xylene microcosms but not in the toluene microcosm. Detection of TOL at gasoline-contaminated sites may result mainly from the m-xylene and p-xylene fractions of gasoline.

TABLE 22
Summary of the Detection of Aromatic Oxygenase Genes at Field Sites.

| Monitoring | | Total BTEX | | | | | | |
|---|---|---|---|---|---|---|---|---|
| Well | Compounds | (ug L⁻¹) | PHE | RMO | NAH | TOL | BPH4 | TOD |
| F-OW-5 | BTEX | 77100 | PHE | RMO | NAH | TOL | BPH4 | TOD |
| F-OW-24 | BTEX | 52000 | PHE | RMO | NAH | TOL | | TOD |
| F-RW-1 | BTEX | 34800 | PHE | RMO | NAH | TOL | | TOD |
| F-OW-17 | BTX | 9600 | PHE | RMO | NAH | | | |
| F-OW-18 | BEX | 6740 | PHE | RMO | NAH | | BPH4 | |
| W-MW-11 | BTEX | 3880 | PHE | | NAH | TOL | | |
| F-OW-23 | BEX | 1185 | PHE | RMO | NAH | TOL | | TOD |
| W-MW-5 | BE | 296 | | | | | | |
| F-OW-12 | B | 190 | PHE | RMO | | | BPH4 | |
| W-MW-2 | BEX | 152 | PHE | RMO | NAH | | | |
| W-MW-12 | B | 26 | PHE | RMO | NAH | | | |
| W-MW-3 | (X) | | PHE | RMO | NAH | | | |
| W-MW-7 | (X) | | PHE | RMO | NAH | | | |
| F-OW-19 | nd | | PHE | RMO | | | | |
| F-OW-20 | nd | | PHE | RMO | NAH | | | |
| F-OW-21 | nd | | PHE | RMO | NAH | TOL | | |
| F-OW-22 | nd | | PHE | RMO | | | | |
| F-OW-16 | nd | | | | | | | |
| W-MW-4 | nd | | | | | | | |
| W-MW-6 | nd | | | | | | | |
| W-MW-9 | nd | | | | | | | |
| W-MW-10 | nd | | | | | | | |

(F-) denotes a well at the Frankfort site,
(W-) denotes a well at the Winamac site.
(X) denotes that xylenes were detected at a prior sampling event.
(nd) not detected.

DNA extractions from groundwater samples were screened for the presence of aromatic oxygenase genes by conventional PCR and agarose gel electrophoresis. Real-time PCR was used to quantify oxygenase genes from positive samples. The quantification limit of the real-time PCR assay was 10³ copies reaction⁻¹ which corresponds to 2×10⁴ copies g soil⁻¹. Gene copy numbers observed in contaminated wells were at least 10⁵ copies g soil⁻¹ and more often (in forty-nine of fifty-eight quantifiable samples) on the order of 10⁶ to 10⁹ copies g soil⁻¹. On average, PHE copies g soil⁻¹ were greater than those of other oxygenase genes likely due to the number of substrates that select for PHE-harboring strains. No relationship was evident between copy number and BTX concentration indicating that a factor other than carbon was limiting. Overall, quantification suggests an “on or off” nature in which a particular oxygenase gene is present in copy numbers in excess of 10⁶ copies g soil⁻¹ or it is not selected and thus not detected. The “on or off” nature is also evident considering the little variability about the average PHE, RMO, and NAH copies g soil⁻¹ in contaminated wells. Enumeration of oxygenase genes in wells immediately downgradient but with non-detectable BTX concentrations were similar to those of wells within the plume indicating that the aromatic hydrocarbon-degrading population was also present near the edge of the plume (FIG. 10). In the impacted wells (wells containing BTX and those immediately downgradient) copy numbers of PHE, RMO, NAH, and TOL are approximately 10⁷ copies g soil⁻¹ but none were detected in sentinel wells. In terms of site assessment therefore, detection of aromatic oxygenase genes by conventional PCR may be adequate to document biodegradation potential.

Incorporation of microbial characterization (16S rDNA PCR-DGGE and PCR amplfication of aromatic oxygenase genes) into the site management plan provided supporting evidence of natural attenuation of the sites investigated here and could be used for optimization of engineered remediation systems. PCR-DGGE profiles of contaminated well samples showed multiple, bright bands compared to the smear of products in upgradient wells suggesting the enrichment of subpopulations of the indigenous population as a result of gasoline-contamination. PCR detection and enumeration of aromatic oxygenase genes clearly indicated the presence of bacteria capable of biodegrading aromatic hydrocarbons, the contaminants of principal concern. PHE, RMO, and NAH were routinely detected in impacted wells. TOL and TOD were detected primarily in areas with high BTX concentrations. The detection of naphthalene and biphenyl dioxygenase genes at gasoline contaminated sites may indicate that these pathways are more broadly applicable than currently known and deserves further attention. A more thorough understanding of the selection of aromatic catabolic pathways may improve prediction of complex mixtures and in turn improve managing bioremediation in the field.

### SUMMARY OF EXPERIMENTAL RESULTS

In the microcosm study, no oxygenase genes were consistently detected in the samples from the unamended microcosm, however, aromatic catabolic oxygenase genes were detected in virtually all enrichment microcosms. PHE, RMO, and TOL primer sets were all detected in the gasoline microcosm and therefore are likely to be good indicators of bioremediation potential at gasoline contaminated sites. The PHE primer set is expected to be a particularly important indicator. First, PHE was consistently detected in the benzene microcosm even when low substrate concentrations were maintained. Considering the high solubility, high toxicity, and corresponding low maximum contaminant level (MCL) for benzene, the ability to detect a catabolic genotype involved in benzene biodegradation is critical for field applications. Second, PHE was detected in naphthalene and biphenyl microcosm samples and along with NAH may be a good indicator of biodegradation potential at diesel of PAH-contaminated sites. From the o-xylene microcosm results, RMO may be enriched by the o-xylene fraction of gasoline and may play an important role in the biodegradation of this compound at gasoline-contaminated sites. TOL was detected only in the m-xylene and p-xylene microcosms indicating that TOL may be more involved in the catabolism of these xylene isomers than toluene. Thus, the m-xylene and p-xylene fractions of gasoline may have enriched for TOL-harboring bacteria in the gasoline microcosm.

While microcosm experiments gave insight into the selection of aromatic catabolic pathways and indicated that PCR amplification would allow detection of these genotypes in the environment, many environmental factors cannot be duplicated in the laboratory. In order to be fully validated, the methods developed had to be tested at the field scale. The first site tested was an operating gasoline and diesel fuel station located in Frankfort, Ind. Groundwater BTEX levels at this site ranged from over 50 mg/l to non-detectable in the outlying wells. The second site was an INDOT facility in Winamac, Ind. which had suffered gasoline and diesel contamination resulting from leaking underground storage tanks. The Winamac site has been undergoing MNA, current BTX levels are low, and was chosen to evaluate a system near closure. At the Winamac site high copy numbers of aromatic oxygenase genes were enumerated in impacted wells but none were detected in “sentinel wells” outside the plume. Aromatic oxygenase genes were also detected at Frankfort in the contaminated and downgradient wells but not in the upgradient well. Overall, the integration of chemical and genetic analysis gave a more clear indication of on-going remediation at the sites.

PHE and RMO were detected in nearly all impacted wells and those on the edge of the contaminant plume at both sites. In the microcosm study, PHE was consistently detected in the benzene, toluene, p-xylene, naphthalene, biphenyl, and gasoline microcosms. RMO was detected in the o-xylene and gasoline microcosms. The prevalence of these genotypes in the field may therefore stem from the abundance of the wide variety of acceptable substrates present in gasoline; thus these genotypes may be very important for biodegradation in the field. Conversely, TOL and TOD were primarily observed in well samples with high BTEX concentrations. While host strain-specific factors cannot be ignored the results suggest that ring-hydroxylating pathways for BTEX catabolism may confer a competitive advantage under certain field conditions. NAH and BPH4 were also observed in field samples raising the question of their role in biodegradation of gasoline constituents. Coupled with chemical data, enumeration of aromatic oxygenase genes at these sites provided strong evidence of biodegradation of the targeted aromatic hydrocarbons.

### CLOSURE

While the invention has been illustrated and described in detail in the drawings and foregoing description, the same is to be considered as illustrative and not restrictive in character. Only certain embodiments have been shown and described, and all changes, equivalents, and modifications that come within the spirit of the invention described herein are desired to be protected. Any experiments, experimental examples, or experimental results provided herein are intended to be illustrative of the present invention and should not be considered limiting or restrictive with regard to the invention scope. Further, any theory, mechanism of operation, proof, or finding stated herein is meant to further enhance understanding of the present invention and is not intended to limit the present invention in any way to such theory, mechanism of operation, proof, or finding. Thus, the specifics of this description should not be interpreted to limit the scope of this invention to the specifics thereof. Rather, the scope of this invention should be evaluated with reference to the claims appended hereto. In reading the claims it is intended that when words such as “a”, “an”, “at least one”, and “at least a portion” are used there is no intention to limit the claims to only one item unless specifically stated to the contrary in the claims. Further, when the language “at least a portion” and/or “a portion” is used, the claims may include a portion and/or the entire items unless specifically stated to the contrary. Finally, all patents, patent applications and publications, including electronically available material such as GenBank submissions, cited in this specification are herein incorporated by reference to the extent not inconsistent with the present disclosure as if each were specifically and individually indicated to be incorporated by reference and set forth in its entirety herein.

What is claimed is:

1. A method for assessing the bioremediation potential of a microbial community in a soil or water sample, comprising:
providing a plurality of PCR primer sets, wherein each set corresponds to a distinct family or subfamily of functional aromatic oxygenase genes and is effective to selectively amplify target regions from diverse aromatic oxygenase genes in the corresponding family or subfamily;
providing a mixture of polynucleotides isolated from microbes present in a soil or water sample;
performing one or more quantitative PCR amplification reactions using the primer sets to quantify diverse aromatic oxygenase genes of each corresponding family or subfamily in the mixture; and
determining the bioremediation potential of microbes in the sample based upon results of the one or more quantitative PCR reactions.

2. The method in accordance with claim 1 wherein the sample is contaminated with a plurality of aromatic pollutants.

3. The method in accordance with claim 1 wherein the sample is a sample from a petroleum contaminated site.

4. The method in accordance with claim 1 wherein the plurality of primer sets includes at least two primer sets corresponding to two families or subfamilies of functional aromatic oxygenase genes that encode enzymes having specificity for different aromatic pollutant compounds.

5. The method in accordance with claim 1 wherein said performing comprises performing real-time quantitative PCR analysis.

6. The method in accordance with claim 5 wherein the real-time quantitative PCR analysis is performed using a double stranded DNA-binding dye.

7. The method in accordance with claim 6 wherein the dye is a SYBR Green dye.

8. The method in accordance with claim 5 wherein the real-time quantitative PCR analysis is performed using a member selected from the group consisting of molecular beacons, hybridization probes and hydrolysis probes; and wherein the member is effective to hybridize to a polynucleotide segment of from about 10 to about 40 bases that is conserved in the members of each family or subfamily.

9. The method in accordance with claim 1 wherein the plurality of primer sets includes at least two primer sets corresponding to two families or subfamilies of functional aromatic oxygenase genes that encode enzymes having specificity for the same aromatic pollutant compound.

10. The method in accordance with claim 1 wherein at least one of the one or more quantitative PCR amplification reactions comprises a multiplex real-time quantitative PCR reaction.

11. The method in accordance with claim 10 wherein the plurality of primer sets includes a first primer set that is effective to selectively amplify a family or subfamily of phenol monooxygenase genes and a second primer set that is effective to selectively amplify a family or subfamily of naphthalene dioxygenase genes; and wherein the first and second primer sets are used together to amplify diverse target regions in a multiplex real-time quantitative PCR reaction.

12. The method in accordance with claim 11 wherein the first primer set comprises a forward primer having the nucleotide sequence of SEQ ID NO: 18 and a reverse primer having the nucleotide sequence of SEQ ID NO: 19.

13. The method in accordance with claim 11 wherein the second primer set comprises a forward primer having the nucleotide sequence of SEQ ID NO: 1 and a reverse primer having the nucleotide sequence of SEQ ID NO: 2.

14. The method in accordance with claim 10 wherein the plurality of primer sets includes a first primer set that is effective to selectively amplify a family or subfamily of xylene monooxygenase genes and a second primer set that is effective to selectively amplify a family or subfamily of toluene dioxygenase genes; and wherein the first and second primer sets are used together to amplify diverse target regions in a multiplex real-time quantitative PCR reaction.

15. The method in accordance with claim 14 wherein the first primer set comprises a forward primer having the nucleotide sequence of SEQ ID NO: 5 and a reverse primer having the nucleotide sequence of SEQ ID NO: 6.

16. The method in accordance with claim 14 wherein the second primer set comprises a forward primer having the nucleotide sequence of SEQ ID NO: 3 and a reverse primer having the nucleotide sequence of SEQ ID NO: 4.

17. The method in accordance with claim 10 wherein the plurality of primer sets includes a first primer set that is effective to selectively amplify a first subfamily of biphenyl dioxygenase genes and a second primer set that is effective to selectively amplify a second subfamily of biphenyl dioxygenase genes; and wherein the first and second primer sets are used together to amplify diverse target regions in a multiplex real-time quantitative PCR reaction.

18. The method in accordance with claim 17 wherein the first primer set comprises a forward primer having the nucleotide sequence of SEQ ID NO: 9 and a reverse primer having the nucleotide sequence of SEQ ID NO: 10.

19. The method in accordance with claim 17 wherein the second primer set comprises a forward primer having the nucleotide sequence of SEQ ID NO: 12 and a reverse primer having the nucleotide sequence of SEQ ID NO: 13.

20. The method in accordance with claim 1 wherein the plurality of primer sets includes at least two primer sets, each of which is effective to selectively amplify a family or subfamily of functional aromatic oxygenase genes selected from the group consisting of naphthalene dioxygenase genes, toluene dioxygenase genes, xylene monooxygenase genes, biphenyl dioxygenase genes, toluene monooxygenase genes and phenol monooxygenase genes.

21. The method in accordance with claim 1 wherein the plurality of primer sets includes at least one primer set selected from the group consisting of:
a set comprising a forward primer having the nucleotide sequence of SEQ ID NO: 1 and a reverse primer having the nucleotide sequence of SEQ ID NO: 2;
a set comprising a forward primer having the nucleotide sequence of SEQ ID NO: 3 and a reverse primer having the nucleotide sequence of SEQ ID NO: 4;
a set comprising a forward primer having the nucleotide sequence of SEQ ID NO: 5 and a reverse primer having the nucleotide sequence of SEQ ID NO: 6;
a set comprising a forward primer having the nucleotide sequence of SEQ ID NO: 7 and a reverse primer having the nucleotide sequence of SEQ ID NO: 8;
a set comprising a forward primer having the nucleotide sequence of SEQ ID NO: 9 and a reverse primer having the nucleotide sequence of SEQ ID NO: 10;
a set comprising a forward primer having the nucleotide sequence of SEQ ID NO: 11 and a reverse primer having the nucleotide sequence of SEQ ID NO: 13;
a set comprising a forward primer having the nucleotide sequence of SEQ ID NO: 12 and a reverse primer having the nucleotide sequence of SEQ ID NO: 13;
a set comprising a forward primer having the nucleotide sequence of SEQ ID NO: 14 and a reverse primer having the nucleotide sequence of SEQ ID NO: 15;
a set comprising a forward primer having the nucleotide sequence of SEQ ID NO: 16 and a reverse primer having the nucleotide sequence of SEQ ID NO: 17; and
a set comprising a forward primer having the nucleotide sequence of SEQ ID NO: 18 and a reverse primer having the nucleotide sequence of SEQ ID NO: 19.

22. The method in accordance with claim 1 wherein the plurality of primer sets includes at least two primer sets selected from the group consisting of:
a set comprising a forward primer having the nucleotide sequence of SEQ ID NO: 1 and a reverse primer having the nucleotide sequence of SEQ ID NO: 2;
a set comprising a forward primer having the nucleotide sequence of SEQ ID NO: 3 and a reverse primer having the nucleotide sequence of SEQ ID NO: 4;
a set comprising a forward primer having the nucleotide sequence of SEQ ID NO: 5 and a reverse primer having the nucleotide sequence of SEQ ID NO: 6;
a set comprising a forward primer having the nucleotide sequence of SEQ ID NO: 7 and a reverse primer having the nucleotide sequence of SEQ ID NO: 8;
a set comprising a forward primer having the nucleotide sequence of SEQ ID NO: 9 and a reverse primer having the nucleotide sequence of SEQ ID NO: 10;
a set comprising a forward primer having the nucleotide sequence of SEQ ID NO: 11 and a reverse primer having the nucleotide sequence of SEQ ID NO: 13;
a set comprising a forward primer having the nucleotide sequence of SEQ ID NO: 12 and a reverse primer having the nucleotide sequence of SEQ ID NO: 13;
a set comprising a forward primer having the nucleotide sequence of SEQ ID NO: 14 and a reverse primer having the nucleotide sequence of SEQ ID NO: 15;
a set comprising a forward primer having the nucleotide sequence of SEQ ID NO: 16 and a reverse primer having the nucleotide sequence of SEQ ID NO: 17; and
a set comprising a forward primer having the nucleotide sequence of SEQ ID NO: 18 and a reverse primer having the nucleotide sequence of SEQ ID NO: 19.

23. The method in accordance with claim 1 wherein said performing comprises performing real-time quantitative PCR analysis of the mixture using each of the following primer sets:
a set comprising a forward primer having the nucleotide sequence of SEQ ID NO: 1 and a reverse primer having the nucleotide sequence of SEQ ID NO: 2;
a set comprising a forward primer having the nucleotide sequence of SEQ ID NO: 3 and a reverse primer having the nucleotide sequence of SEQ ID NO: 4;
a set comprising a forward primer having the nucleotide sequence of SEQ ID NO: 5 and a reverse primer having the nucleotide sequence of SEQ ID NO: 6;
a set comprising a forward primer having the nucleotide sequence of SEQ ID NO: 7 and a reverse primer having the nucleotide sequence of SEQ ID NO: 8;
a set comprising a forward primer having the nucleotide sequence of SEQ ID NO: 9 and a reverse primer having the nucleotide sequence of SEQ ID NO: 10;
a set comprising a forward primer having the nucleotide sequence of SEQ ID NO: 11 and a reverse primer having the nucleotide sequence of SEQ ID NO: 13;
a set comprising a forward primer having the nucleotide sequence of SEQ ID NO: 12 and a reverse primer having the nucleotide sequence of SEQ ID NO: 13;
a set comprising a forward primer having the nucleotide sequence of SEQ ID NO: 14 and a reverse primer having the nucleotide sequence of SEQ ID NO: 15;
a set comprising a forward primer having the nucleotide sequence of SEQ ID NO: 16 and a reverse primer having the nucleotide sequence of SEQ ID NO: 17; and
a set comprising a forward primer having the nucleotide sequence of SEQ ID NO: 18 and a reverse primer having the nucleotide sequence of SEQ ID NO: 19.

24. A screening protocol for detecting and quantifying multiple families or subfamilies of functional aromatic oxygenase genes of diverse aromatic pollutant-degrading microbial species in a soil or water sample, comprising:
providing a mixture of polynucleotides isolated from microbes present in a soil or water sample; and
performing quantitative PCR analysis of the mixture using a plurality of primer sets configured to selectively amplify different families or subfamilies of functional aromatic oxygenase genes.

25. The protocol in accordance with claim 24 wherein a plurality of the primer sets are suitable for use together in a multiplex real-time PCR reaction.

26. The protocol in accordance with claim 24 wherein each of the primer sets is used in separate real-time quantitative PCR reactions to separately quantify each corresponding family or subfamily of functional aromatic oxygenase genes.

27. A method of monitoring the bioremediation potential of a microbial community in a soil or water system contaminated with aromatic pollutants, comprising:
providing a mixture of polynucleotides isolated from a soil or water sample corresponding to the system; and
performing quantitative PCR analysis of said mixture using a plurality of primer sets configured to selectively amplify target segments from corresponding families or subfamilies of aromatic oxygenase genes to provide a quantity value corresponding to aromatic oxygenase gene abundance in the sample;
wherein the aromatic oxygenase gene abundance correlates with the bioremediation potential of the sample.

28. The method in accordance with claim 27, further comprising perturbing the system, waiting a period of time sufficient to allow the microbial community in the system to respond to said perturbing, and repeating said providing and performing to determine whether the bioremediation potential of the sample has changed.

29. The method in accordance with claim 27 wherein said quantitative PCR is competitive, noncompetitive, kinetic, or combinations thereof.

30. The method in accordance with claim 27 wherein the mixture of polynucleotides comprises a mixture of RNA polynucleotides and wherein said performing comprises performing quantitative RT-PCR on said RNA using a plurality of primer sets configured to selectively amplify target segments from corresponding families or subfamilies of mRNA corresponding to aromatic oxygenase genes to provide a quantity value corresponding to aromatic oxygenase gene expression in the sample.

31. The method in accordance with claim 30 wherein said quantitative RT-PCR is competitive, noncompetitive, kinetic, or combinations thereof.

32. A real-time Polymerase Chain Reaction (PCR) method for the selective detection and quantification of diverse families or subfamilies of aromatic oxygenase genes, each family or subfamily including a unique conserved region or a plurality of unique conserved sub-regions, said method comprising:
providing a mixture of polynucleotides isolated from a soil or water sample;
providing a plurality of primer sets configured to selectively amplify target segments from corresponding families or subfamilies of aromatic oxygenase genes and
performing quantitative PCR analysis of said mixture using the plurality of primer sets to provide a quantity value corresponding to aromatic oxygenase gene abundance in the sample.

33. The real-time PCR method in accordance with claim 32 wherein the PCR method comprises at least one polymerization reaction performed by adding two or more primer sets to the same PCR mixture, each primer set being specific for a single family or subfamily of aromatic oxygenase genes.

34. The real-time PCR method according to claim 32 wherein each of the primer sets is effective for amplifying a target segment from a different family or subfamily including aromatic oxygenase genes selected from the group consisting of a naphthalene dioxygenase genes, toluene dioxygenase genes, xylene monooxygenase genes, biphenyl dioxygenase genes, toluene monooxygenase genes and phenol monooxygenase genes.

35. The real-time PCR method according to claim 32 wherein at least one of the primer sets is selected from the group consisting of:
a set comprising a forward primer having the nucleotide sequence of SEQ ID NO: 1 and a reverse primer having the nucleotide sequence of SEQ ID NO: 2;
a set comprising a forward primer having the nucleotide sequence of SEQ ID NO: 3 and a reverse primer having the nucleotide sequence of SEQ ID NO: 4;
a set comprising a forward primer having the nucleotide sequence of SEQ ID NO: 5 and a reverse primer having the nucleotide sequence of SEQ ID NO: 6;
a set comprising a forward primer having the nucleotide sequence of SEQ ID NO: 7 and a reverse primer having the nucleotide sequence of SEQ ID NO: 8;
a set comprising a forward primer having the nucleotide sequence of SEQ ID NO: 9 and a reverse primer having the nucleotide sequence of SEQ ID NO: 10;
a set comprising a forward primer having the nucleotide sequence of SEQ ID NO: 11 and a reverse primer having the nucleotide sequence of SEQ ID NO: 13;
a set comprising a forward primer having the nucleotide sequence of SEQ ID NO: 12 and a reverse primer having the nucleotide sequence of SEQ ID NO: 13;
a set comprising a forward primer having the nucleotide sequence of SEQ ID NO: 14 and a reverse primer having the nucleotide sequence of SEQ ID NO: 15;
a set comprising a forward primer having the nucleotide sequence of SEQ ID NO: 16 and a reverse primer having the nucleotide sequence of SEQ ID NO: 17; and
a set comprising a forward primer having the nucleotide sequence of SEQ ID NO: 18 and a reverse primer having the nucleotide sequence of SEQ ID NO: 19.

36. The real-time PCR method according claim 32 wherein said PCR is a reverse-transcription (RT) PCR.

37. The real-time PCR method according to claim 32 wherein in the same one-tube reaction a standard nucleic acid sequence is simultaneously amplified and quantified according to real-time PCR principles; and wherein the standard nucleic acid sequence is added in a known copy number to a sample to be tested.

38. The real-time PCR method according to claim 32 wherein a double stranded DNA-binding dye is added to a sample to be tested.

39. The real-time PCR method according to claim 38 wherein primer sets corresponding to two or more families or subfamilies of aromatic oxygenase genes are included a single one-tube reaction for simultaneous amplification and quantification of the two or more families or subfamilies.

40. A primer set selected from the group consisting of:
a set comprising a forward primer having the nucleotide sequence of SEQ ID NO: 1 and a reverse primer having the nucleotide sequence of SEQ ID NO: 2;
a set comprising a forward primer having the nucleotide sequence of SEQ ID NO: 3 and a reverse primer having the nucleotide sequence of SEQ ID NO: 4;
a set comprising a forward primer having the nucleotide sequence of SEQ ID NO: 5 and a reverse primer having the nucleotide sequence of SEQ ID NO: 6;
a set comprising a forward primer having the nucleotide sequence of SEQ ID NO: 7 and a reverse primer having the nucleotide sequence of SEQ ID NO: 8;
a set comprising a forward primer having the nucleotide sequence of SEQ ID NO: 9 and a reverse primer having the nucleotide sequence of SEQ ID NO: 10;
a set comprising a forward primer having the nucleotide sequence of SEQ ID NO: 11 and a reverse primer having the nucleotide sequence of SEQ ID NO: 13;
a set comprising a forward primer having the nucleotide sequence of SEQ ID NO: 12 and a reverse primer having the nucleotide sequence of SEQ ID NO: 13;
a set comprising a forward primer having the nucleotide sequence of SEQ ID NO: 14 and a reverse primer having the nucleotide sequence of SEQ ID NO: 15;
a set comprising a forward primer having the nucleotide sequence of SEQ ID NO: 16 and a reverse primer having the nucleotide sequence of SEQ ID NO: 17; and
a set comprising a forward primer having the nucleotide sequence of SEQ ID NO: 18 and a reverse primer having the nucleotide sequence of SEQ ID NO: 19.

41. A primer set pair for performing multiplex real-time quantitative PCR comprising a forward primer having the nucleotide sequence of SEQ ID NO: 18, a reverse primer having the nucleotide sequence of SEQ ID NO: 19, a forward primer having the nucleotide sequence of SEQ ID NO: 1, and a reverse primer having the nucleotide sequence of SEQ ID NO: 2.

42. A primer set pair for performing multiplex real-time quantitative PCR comprising a forward primer having the nucleotide sequence of SEQ ID NO: 5, a reverse primer having the nucleotide sequence of SEQ ID NO: 6, a forward primer having the nucleotide sequence of SEQ ID NO: 3, and a reverse primer having the nucleotide sequence of SEQ ID NO: 4.

43. A primer set pair for performing multiplex real-time quantitative PCR comprising a forward primer having the nucleotide sequence of SEQ ID NO: 9, a reverse primer having the nucleotide sequence of SEQ ID NO: 10, a forward primer having the nucleotide sequence of SEQ ID NO: 12, a reverse primer having the nucleotide sequence of SEQ ID NO: 13.

44. A method for making a series of PCR primer sets for use in determining bioremediation potential of microbes in a sample to be analyzed, comprising:
identifying a plurality of aromatic pollutants for which bioremediation potential is to be determined;
preparing an alignment of functional aromatic oxygenase genes for each group of oxygenase genes having specificity for one of the pollutants; wherein each of the alignments includes genes from diverse species that encode oxygenase enzymes effective to oxygenate the corresponding aromatic pollutant;
identifying a region of each alignment comprising from about 50 to about 1000 bases that is substantially conserved or that includes two or more sub-regions that are substantially conserved in a plurality of the genes in the alignment; and
preparing a series of primer sets, each primer set corresponding to one alignment and comprising a forward primer of from about 10 to about 40 bases complementary to a nucleotide segment of a first strand of the region and a reverse primer of from about 10 to about 40 bases complementary to a nucleotide segment of a second strand of the region;
wherein the forward and reverse primers corresponding to each alignment span a target region in each of the plurality of genes; and
wherein each primer set is effective to amplify the target regions from the plurality of genes when present in the sample by quantitative PCR.
